SEQUENCE LISTING (120 sequences)

SEQ ID NO: 1 - DNA, 730 bp, Medicago truncatula
atgacttcctctgctagtaaattctataccatcttcatttttgtctgccttgcctttctctttatttccacatctggtacaaattaatcaaataacctatgttaataatttctcatttattaatccttatttatgttcttaatttgcttcattgttattttcttgctttctatgtgtagaggtggaagcaaaactttgtcaaaagcgaagtacaacatggtcaggaccttgtcttaacacaggaaactgcaaaagacaatgcattaatgtggagcatgctacttttggtgcttgtcatcgtcaaggctttggttttgcttgcttctgctacaaaaaatgtgctccaaagagtatattgttaacattattctttaaaacgattattaaaatgccaaaactaaaatataacaatataaatgtaacaattaatcattatgatcgatgcatatcacctccagatatatgtggtacaaataatcaaacaatctatattaataattgttcattttctaatccatatttttgtgtttttaatttttcatatttttttttttttttatatgtagaggtggaacctaaactttgtgaaaggcgaagcaaaacatggtcaggaccttgtcttatctcaggaaattgtaaaagacagtgcatcaatgttgagcatgcaacttctggtgcttgtcaccgtcaaggcattggttttgcttgcttctgcaagaaaaaatgttga

SEQ ID NO: 2 - protein, 136 aa, Medicago truncatula
MTSSASKFYTIFIFVCLAFLFISTSEVEAKLCQKRSTTWSGPCLNTGNCKRQCINVEHATFGACHRQGFGFACFCYKKCAPKKVEPKLCERRSKTWSGPCLISGNCKRQCINVEHATSGACHRQGIGFACFCKKKC

SEQ ID NO: 3 - protein, 29 aa, Medicago truncatula
MTSSASKFYTIFIFVCLAFLFISTSEVEA

SEQ ID NO: 4 - protein, 50 aa, Medicago truncatula
KLCQKRSTTWSGPCLNTGNCKRQCINVEHATFGACHRQGFGFACFCYKKC

SEQ ID NO: 5 - protein, 50 aa, Medicago truncatula
KLCERRSKTWSGPCLISGNCKRQCINVEHATSGACHRQGIGFACFCKKKC

SEQ ID NO: 6 - protein, 7 aa, Medicago truncatula
APKKVEP

SEQ ID NO: 7 - protein, 105 aa, Medicago truncatula
CQKRSTTWSGPCLNTGNCKRQCINVEHATFGACHRQGFGFACFCYKKCAPKKVEPKLCERRSKTWSGPCLISGNCKRQCINVEHATSGACHRQGIGFACFCKKKC

SEQ ID NO: 8 - protein, 50 aa, Medicago truncatula
KVCQKRSKTWSGPCLNTGNCKRQCVDVENATFGACHRQGFGFACFCYKKC

SEQ ID NO: 9 - protein, 51 aa, Cicer arietinum
KLCQRRSKTWSGPCIITGNCKNQCKNVEHATFGACHRQGFGFACFCYFNCH

SEQ ID NO: 10 - protein, 50 aa, Medicago truncatula
NICKRKSTTWSGPCLNTGNCKNQCINVEHATFGACHQDGFGFACFCYFNC

SEQ ID NO: 11 - protein, 51 aa, Medicago truncatula
NTCQRKSKTWSGPCLNTANCKNQCISKEPPATFGACHRDGIGFACFCYFNC

SEQ ID NO: 12 - protein, 50 aa, Nicotiana sylvestris
KVCQRRSKTWSGPCINTGNCSRQCKNQEDGRFGACHRSGIGFACFCYFNC

SEQ ID NO: 13 - protein, 50 aa, Solanum lycopersicum
KVCQRRSKTWSGPCINTGNCSRQCKQQEDARFGACHRSGFGFACFCYFKC

SEQ ID NO: 14 - protein, 50 aa, Citrus sinensis
KVCQLRSKTWSGLCLNTGNCSRQCKQQEDARFGACHRQGIGFACFCYFKC

SEQ ID NO: 15 - protein, 50 aa, Citrus clementina
KVCQRRSKTWSGLCLNTGNCSRQCKQQEDARFGACHRQGIGFACFCYFKC

SEQ ID NO: 16 - protein, 51 aa, Solanum tuberosum
KVCQRRSQTWSGMCINTGNCSRQCKQQEDARFGACHQNGIGFACFCYFTCK

SEQ ID NO: 17 - protein, 50 aa, Citrus sinensis
KVCQRRSKTWSGPCLNTGKCSRHCKQQEDARYGACYRQGTGYACFCYFEC

SEQ ID NO: 18 - protein, 50 aa, Citrus clementina
KVCQRRSKTWSGPCLNTGKCSRQCKQQEYARYGACYRQGAGYACYCYFNC

SEQ ID NO: 19 - protein, 50 aa, Citrus clementina; identical to SEQ ID NO: 18

SEQ ID NO: 20 - protein, 50 aa, Amborella trichopoda
KLCQKRSRTWSGFCANSNNCSRQCKNLEGARFGACHRQRIGLACFCYFNC

SEQ ID NO: 21 - protein, 50 aa, Sesamum indicum
KICQRMSKTWSGVCLNSGNCDRQCRNWERAQHGACHRRGLGFACLCYFKC

SEQ ID NO: 22 - protein, 50 aa, Arabidopsis thaliana
RICERRSKTWTGFCGNTRGCDSQCKRWERASHGACHAQFPGFACFCYFNC

SEQ ID NO: 23 - protein, 50 aa, Arabidopsis halleri
RICERRSKTWTGFCGNTRGCDSQCKSWERASHGACHAQFPGFACFCYFNC

SEQ ID NO: 24 - protein, 50 aa, Arabidopsis lyrata; identical to SEQ ID NO: 23

SEQ ID NO: 25 - protein, 50 aa, Capsella rubella
RICQRRSKTWTGFCGNTRGCDSQCKRWERASHGACHAQFPGFACFCYFNC

SEQ ID NO: 26 - protein, 50 aa, Eutrema salsugineum
RVCERRSKTWTGFCGNTRGCDSQCKRWERASHGACHAQFPGFACFCYFNC

SEQ ID NO: 27 - protein, 50 aa, Brassica rapa
RVCQRRSKTWTGFCGNTRGCDSQCKRWERASHGACHAQFPGFACFCYFNC

SEQ ID NO: 28 - protein, 50 aa, Brassica napus; identical to SEQ ID NO: 27

SEQ ID NO: 29 - protein, 50 aa, Brassica napus; identical to SEQ ID NO: 27

SEQ ID NO: 30 - protein, 50 aa, Arabis alpina
RICERRSKTWTGFCANTRGCDSQCKRWERASHGACHAQFPGVACFCYFNC

SEQ ID NO: 31 - protein, 50 aa, Camelina sativa
RTCERRSKTWTGFCGNTRGCDSQCRRWEHASHGACHAQFPGFACFCYFNC

SEQ ID NO: 32 - protein, 50 aa, Camelina sativa
RTCERRSKTWTGFCGNTRGCDSQCRSWEGASHGACHAQFPGFACFCYFNC

SEQ ID NO: 33 - protein, 50 aa, Erythranthe guttata
RLCERRSKTWTGFCGSSNNCNNQCRNWERASHGACHAQFPGFACFCYFNC

SEQ ID NO: 34 - protein, 50 aa, Ricinus communis
KVCQRRSKTWSGFCGSTKNCDRQCKNWEGALHGACHAQFPGVACFCYFKC

SEQ ID NO: 35 - protein, 50 aa, Vitis vinifera
KVCQRPSKTWSGFCGSSKNCDRQCKNWEGAKHGACHAKFPGVACFCYFNC

SEQ ID NO: 36 - protein, 50 aa, Cucumis sativus
KVCERRSKTWSGWCGNTKHCDRQCKNWEGATHGACHAQFPGRACFCYFNC

SEQ ID NO: 37 - protein, 50 aa, Pyrus bretschneideri
RICQRRSKTWSGFCANTGNCNRQCTNWEGALHGACHAQFPGVACFCYFRC

SEQ ID NO: 38 - protein, 50 aa, Prunus persica
RICQRRSKTWSGFCGNTGNCNRQCRNWEGALRGACHAQSPGFACFCYFRC

SEQ ID NO: 39 - protein, 107 aa, Artificial
  synthetic
KLCQAASTTWSGPCLNTGNCKRQCINVEHATFGACAAQGFGFACFCYKKCAPKKVEPKLCEAASKTWSGPCLISGNCKRQCINVEHATSGACAAQGIGFACFCKKKC

SEQ ID NO: 40 - protein, 50 aa, Fragaria vesca
RICQRRSKTWTGLCANTGNCHRQCRNWEGAQRGACHAQFPGFACFCYFNC

SEQ ID NO: 41 - protein, 50 aa, Jatropha curcas
KLCQRRSKTWSGFCGDPGKCNRQCRNWEGASHGACHAQFPGFACFCYFKC

SEQ ID NO: 42 - protein, 50 aa, Amborella trichopoda
KVCQKKSQTWSGECGNTNHCKTQCQKYEDARFGACHSQGFGHACFCYFTC

SEQ ID NO: 43 - protein, 50 aa, Theobroma cacao
KLCQKRSKTWTGPCIKTKNCDHQCRKWEKAQHGACHWQWPGFACFCYVNC

SEQ ID NO: 44 - protein, 50 aa, Medicago truncatula
KLCKRYSTEFHAICTDTGICKNACIYLEHAAFGACHRDGLGFACFCYFNC

SEQ ID NO: 45 - protein, 50 aa, Citrus clementina
KQCSKRAQKWTGPCIKTGSCRNHCRKREGAVDGACHYDFPGFACFCYYNC

SEQ ID NO: 46 - protein, 50 aa, Citrus sinensis; identical to SEQ ID NO: 45

SEQ ID NO: 47 - protein, 49 aa, Solanum lycopersicum
IGCEKMSVTWSGPCFDTGGCNNQCINWEHAIHGACHWDWTGPACYCYFC

SEQ ID NO: 48 - protein, 50 aa, Glycine max
GLCAKRSKTWSGWCGSSNNCDKQCRTKEGATHGACHGNILKRACDCYFKC

SEQ ID NO: 49 - protein, 51 aa, Capsella rubella
AGVCQRYSGSWTGICMSTSNCNTQCIERESAKYGACHFDGNGSACFCYFDC

SEQ ID NO: 50 - protein, 50 aa, Brassica napus
EVCQRYSGSWSGICLSSSNCNTQCIEREHAKYGACHSDDNGLACFCYFDC

SEQ ID NO: 51 - protein, 50 aa, Brassica rapa; identical to SEQ ID NO: 50

SEQ ID NO: 52 - protein, 50 aa, Brassica napus; identical to SEQ ID NO: 50

SEQ ID NO: 53 - protein, 50 aa, Brassica oleracea; identical to SEQ ID NO: 50

SEQ ID NO: 54 - protein, 50 aa, Arabidopsis lyrata
GVCQRYSGSWEGVCIFSSNCNTQCIERESAKYGACHRDDNGLACFCYFDC

SEQ ID NO: 55 - protein, 50 aa, Eutrema salsugineum
EVCQRYSGSWKGFCFSTSNCNNQCIERENAKYGACHADSNGVACFCYFDC

SEQ ID NO: 56 - protein, 173 aa, Leersia perrieri
MEAKAATTVLVLLLLILGGEAGKMCHDPSQTFKGMCFHTMNCISSCTNEGYTGGYCTYLKHKCICTKPCVGEGCEDKICQQHSGTFKGICFNNNNCVSYCVAEQFTSGFCSGVVDRKCICTKECDDKPPTELSLPPPKKKWPPRRQHGQWQQPQRRCDRTEERLCVVWGWLLF

SEQ ID NO: 57 - protein, 103 aa, Leersia perrieri
GKMCHDPSQTFKGMCFHTMNCISSCTNEGYTGGYCTYLKHKCICTKPCVGEGCEDKICQQHSGTFKGICFNNNNCVSYCVAEQFTSGFCSGVVDRKCICTKEC

SEQ ID NO: 58 - protein, 21 aa, Leersia perrieri
MEAKAATTVLVLLLLILGGEA

SEQ ID NO: 59 - protein, 48 aa, Leersia perrieri
GKMCHDPSQTFKGMCFHTMNCISSCTNEGYTGGYCTYLKHKCICTKPC

SEQ ID NO: 60 - protein, 7 aa, Leersia perrieri
VGEGCED

SEQ ID NO: 61 - protein, 48 aa, Leersia perrieri
KICQQHSGTFKGICFNNNNCVSYCVAEQFTSGFCSGVVDRKCICTKEC

SEQ ID NO: 62 - protein, 52 aa, Artificial Sequence
  synthetic
KLCSTTMDLLICGGAIPGAVNQACDDTCRNKGYTGGGFCNMKIQRCVCRKPC

SEQ ID NO: 63 - DNA, 324 bp, Artificial Sequence
  synthetic
aaactttgtcaaaagcgaagtacaacatggtcaggaccttgtcttaacacaggaaactgcaaaagacaatgcattaatgtggagcatgctacttttggtgcttgtcatcgtcaaggctttggttttgcttgcttctgctacaaaaaatgtgctccaaagaaggtggaacctaaactttgtgaaaggcgaagcaaaacatggtcaggaccttgtcttatctcaggaaattgtaaaagacagtgcatcaatgttgagcatgcaacttctggtgcttgtcaccgtcaaggcattggttttgcttgcttctgcaagaaaaaatgttga

SEQ ID NO: 64 - DNA, 327 bp, Artificial Sequence
  synthetic
aagttgtgccaaaaaagatctactacctggagtggtccttgccttaataccggaaattgtaaaagacagtgcatcaatgttgagcatgctacttttggtgcttgtcatagacaaggttttggtttcgcttgtttctgttacaagaaatgtgctccaaagaaagttgaacctaagttgtgtgagagaagatctaaaacttggtctggtccatgtttgatttctggtaactgtaagagacaatgtatcaacgttgaacacgctacaagtggagcctgccatagacaaggtattggtttcgcctgcttttgcaagaagaagtgttaatga

SEQ ID NO: 65 - DNA, 11 bp, Artificial Sequence
  synthetic
tcgagaaaaga

SEQ ID NO: 66 - DNA, 12 bp, Artificial Sequence
  synthetic
taatgagaattc

SEQ ID NO: 67 - DNA, 528 bp, Medicago truncatula
atgacttcctctgctagtaaattctataccatcttcatttttgtctgccttgcctttctctttatttccacatctgaggtggaagcaaaactttgtcaaaagcgaagtacaacatggtcaggaccttgtcttaacacaggaaactgcaaaagacaatgcattaatgtggagcatgctacttttggtgcttgtcatcgtcaaggctttggttttgcttgcttctgctacaaaaaatgtgctccaaagagtatattgttaacattattctttaaaacgattattaaaatgccaaaactaaaatataacaatataaatgtaacaattaatcattatgatcgatgcatatcacctccagatatatgtgaggtggaacctaaactttgtgaaaggcgaagcaaaacatggtcaggaccttgtcttatctcaggaaattgtaaaagacagtgcatcaatgttgagcatgcaacttctggtgcttgtcaccgtcaaggcattggttttgcttgcttctgcaagaaaaaatgttga

SEQ ID NO: 68 - protein, 175 aa, Medicago truncatula
MTSSASKFYTIFIFVCLAFLFISTSEVEAKLCQKRSTTWSGPCLNTGNCKRQCINVEHATFGACHRQGFGFACFCYKKCAPKSILLTLFFKTIIKMPKLKYNNINVTINHYDRCISPPDICEVEPKLCERRSKTWSGPCLISGNCKRQCINVEHATSGACHRQGIGFACFCKKKC

SEQ ID NO: 69 - protein, 146 aa, Medicago truncatula
KLCQKRSTTWSGPCLNTGNCKRQCINVEHATFGACHRQGFGFACFCYKKCAPKSILLTLFFKTIIKMPKLKYNNINVTINHYDRCISPPDICEVEPKLCERRSKTWSGPCLISGNCKRQCINVEHATSGACHRQGIGFACFCKKKC

SEQ ID NO: 70 - protein, 46 aa, Medicago truncatula
APKSILLTLFFKTIIKMPKLKYNNINVTINHYDRCISPPDICEVEP

SEQ ID NO: 71 - DNA, 627 bp, Medicago truncatula
atgacttcctctgctagtaaattctataccatcttcatttttgtctgccttgcctttctctttatttccacatctgaggtggaagcaaaactttgtcaaaagcgaagtacaacatggtcaggaccttgtcttaacacaggaaactgcaaaagacaatgcattaatgtggagcatgctacttttggtgcttgtcatcgtcaaggctttggttttgcttgcttctgctacaaaaaatgtgctccaaagagtatattgttaacattattctttaaaacgattattaaaatgccaaaactaaaatataacaatataaatgtaacaattaatcattatgatcgatgcatatcacctccagatatatgtggtacaaataatcaaacaatctatattaataattgttcattttctaatccatatttttgtgtttttaatttttcatatttttttttttttttatatgtagaggtggaacctaaactttgtgaaaggcgaagcaaaacatggtcaggaccttgtcttatctcaggaaattgtaaaagacagtgcatcaatgttgagcatgcaacttctggtgcttgtcaccgtcaaggcattggttttgcttgcttctgcaagaaaaaatgttga

SEQ ID NO: 72 - protein, 208 aa, Medicago truncatula
MTSSASKFYTIFIFVCLAFLFISTSEVEAKLCQKRSTTWSGPCLNTGNCKRQCINVEHATFGACHRQGFGFACFCYKKCAPKSILLTLFFKTIIKMPKLKYNNINVTINHYDRCISPPDICGTNNQTIYINNCSFSNPYFCVFNFSYFFFFLYVEVEPKLCERRSKTWSGPCLISGNCKRQCINVEHATSGACHRQGIGFACFCKKKC

SEQ ID NO: 73 - protein, 179 aa, Medicago truncatula
KLCQKRSTTWSGPCLNTGNCKRQCINVEHATFGACHRQGFGFACFCYKKCAPKSILLTLFFKTIIKMPKLKYNNINVTINHYDRCISPPDICGTNNQTIYINNCSFSNPYFCVFNFSYFFFFLYVEVEPKLCERRSKTWSGPCLISGNCKRQCINVEHATSGACHRQGIGFACFCKKKC

SEQ ID NO: 74 - protein, 79 aa, Medicago truncatula
APKSILLTLFFKTIIKMPKLKYNNINVTINHYDRCISPPDICGTNNQTIYINNCSFSNPYFCVFNFSYFFFFLYVEVEP

SEQ ID NO: 75 - protein, 76 aa, Medicago truncatula
MARSVPLVSTIFVFLLLLVATGPSMVAEARTCESQSHKFKGPCASDHNCASVCQTERFSGGHCRGFRRRCFCTTHC

SEQ ID NO: 76 - protein, 47 aa, Medicago truncatula
RTCESQSHKFKGPCASDHNCASVCQTERFSGGHCRGFRRRCFCTTHC

SEQ ID NO: 77 - protein, 47 aa, Medicago truncatula; identical to SEQ ID NO: 76

SEQ ID NO: 78 - protein, 72 aa, Medicago sativa
MEKKSLAGLCFLFLVLFVAQEIVVTEARTCENLADKYRGPCFSGCDTHCTTKENAVSGRCRDDFRCWCTKRC

SEQ ID NO: 79 - protein, 45 aa, Medicago sativa
RTCENLADKYRGPCFSGCDTHCTTKENAVSGRCRDDFRCWCTKRC

SEQ ID NO: 80 - protein, 105 aa, Nicotiana alata
MARSLCFMAFAILAMMLFVAYEVQARECKTESNTFPGICITKPPCRKACISEKFTDGHCSKILRRCLCTKPCVFDEKMTKTGAEILAEEAKTLAAALLEEEIMDN

SEQ ID NO: 81 - protein, 47 aa, Nicotiana alata
RECKTESNTFPGICITKPPCRKACISEKFTDGHCSKILRRCLCTKPC

SEQ ID NO: 82 - protein, 105 aa, Solanum lycopersicum
MARSIFFMAFLVLAMMLFVTYEVEAQQICKAPSQTFPGLCFMDSSCRKYCIKEKFTGGHCSKLQRKCLCTKPCVFDKISSEVKATLGEEAKTLSEVVLEEEIMME

SEQ ID NO: 83 - protein, 48 aa, Solanum lycopersicum
QQICKAPSQTFPGLCFMDSSCRKYCIKEKFTGGHCSKLQRKCLCTKPC

SEQ ID NO: 84 - protein, 5 aa, Artificial Sequence
  synthetic
EEKKN

SEQ ID NO: 85 - protein, 5 aa, Artificial Sequence
  synthetic
  MISC_FEATURE (1)..(1): X is E or D
  MISC_FEATURE (2)..(2): X is E or D
  MISC_FEATURE (3)..(3): X is K or R
  MISC_FEATURE (4)..(4): X is K or R
  MISC_FEATURE (5)..(5): X is N or Q
XXXXX

SEQ ID NO: 86 - protein, 31 aa, Impatiens balsamina
DSKPNPTKEEEPAKKPDEVSVKSGGPEVSED

SEQ ID NO: 87 - protein, 28 aa, Impatiens balsamina
ANAEEAAAAIPEASEELAQEEAPVYSED

SEQ ID NO: 88 - protein, 26 aa, Impatiens balsamina
QNAEEAAAAIPEATEKAQEAPVYSED

SEQ ID NO: 89 - protein, 28 aa, Impatiens balsamina
QNAEEAAAAVAIPEASEKAQEGPVYSED

SEQ ID NO: 90 - protein, 16 aa, Impatiens balsamina
SNAADEVATPEDVEPG

SEQ ID NO: 91 - protein, 26 aa, Impatiens balsamina
HNAAEEATLKAFEEEAAREQPVYSED

SEQ ID NO: 92 - protein, 35 aa, Impatiens balsamina
QSAEEAAAFGQGEVTASLMLIMFKACPCMGPVPSV

SEQ ID NO: 93 - protein, 5 aa, Artificial Sequence
  synthetic
GGGGS

SEQ ID NO: 94 - protein, 6 aa, Artificial Sequence
  synthetic
SGGGGS

SEQ ID NO: 95 - protein, 122 aa, Arabidopsis thaliana
MERIPSLASLVSLLIIFATVVNQTRASICNDRLGLCDGCDQRCKAKHGPSCESKCDGPVGMLLCTCTYECGPTKLCNGGLGNCGESCNEQCCDRNCAQRYNGGHGYCNTLDDFSLCLCKYPC

SEQ ID NO: 96 - protein, 44 aa, Arabidopsis thaliana
SICNDRLGLCDGCDQRCKAKHGPSCESKCDGPVGMLLCTCTYEC

SEQ ID NO: 97 - protein, 49 aa, Arabidopsis thaliana
KLCNGGLGNCGESCNEQCCDRNCAQRYNGGHGYCNTLDDFSLCLCKYPC

SEQ ID NO: 98 - protein, 129 aa, Arabidopsis thaliana
METVTSLVFIVNLLIIFTSVVNQARGDTCIDGLGYCNNCDERCKAKHGPSSESSCDRSVGVPLCKCYYECESPPSPPAPPKKCDGGAGICSQRCQGQCCDMNCAQKYIGGHGFCNTLGTFSFCQCEYPC

SEQ ID NO: 99 - protein, 44 aa, Arabidopsis thaliana
DTCIDGLGYCNNCDERCKAKHGPSSESSCDRSVGVPLCKCYYEC

SEQ ID NO: 100 - protein, 10 aa, Arabidopsis thaliana
ESPPSPPAPP

SEQ ID NO: 101 - protein, 49 aa, Arabidopsis thaliana
KKCDGGAGICSQRCQGQCCDMNCAQKYIGGHGFCNTLGTFSFCQCEYPC

SEQ ID NO: 102 - protein, 5 aa, Nicotiana alata
EEKKN

SEQ ID NO: 103 - protein, 18 aa, Artificial Sequence
  synthetic
KESGSVSSEQLAQFRSLD

SEQ ID NO: 104 - protein, 14 aa, Artificial Sequence
  synthetic
EGKSSGSGSESKST

SEQ ID NO: 105 - protein, 12 aa, Artificial Sequence
  synthetic
GSAGSAAGSGEF

SEQ ID NO: 106 - protein, 6 aa, Artificial Sequence
  synthetic
RGFRRR

SEQ ID NO: 107 - protein, 6 aa, Artificial Sequence
  synthetic
KREAEA

SEQ ID NO: 108 - protein, 51 aa, Artificial Sequence
  synthetic
QKLCQRPSGTWSGVCGNNNACKNQCIRLEKARHGSCNYVFPAHKCICYFPC

SEQ ID NO: 109 - protein, 50 aa, Artificial Sequence
  synthetic
ELCEKASKTWSGNCGNTGHCDNQCKSWEGAAHGACHVRNGKHMCFCYFNC

SEQ ID NO: 110 - protein, 46 aa, Artificial Sequence
  synthetic
KTCEHLADTYRGVCFTNASCDDHCKNKAHLISGTCHNWKCFCTQNC

SEQ ID NO: 111 - protein, 136 aa, Medicago sativa
MTSSANKFYTIFVFVCLALLLISTSEVEAKVCQKRSKTWSGPCLNTGNCKRQCINVEHATFGACHRQGFGFACFCYRKCAPKKVEPKLCERRSKTWSGPCLNSGHCKRQCINVEHATSGACHRQGLGIACFCKKKC

SEQ ID NO: 112 - protein, 125 aa, Lotus japonicus
MANKISNSSLFLVFFILVAMVELTMGGGRCSELVSRCGSSFPDCDQTCKSQHSSTNGKGFCNDNICTCYYDCGPPLPPGPVVRKCLAEIGPCSGQCNSNCAGRFKGATGTCNAAFGNLCICQYTC

SEQ ID NO: 113 - DNA, 20 bp, Artificial Sequence
  synthetic ssDNA
ccgtacgttggctgattaca

SEQ ID NO: 114 - DNA, 20 bp, Artificial Sequence
  synthetic ssDNA
ccgtacgttggctgattaca

SEQ ID NO: 115 - DNA, 23 bp, Artificial Sequence
  synthetic ssDNA
cctaaactttgtgaaaggcgaag

SEQ ID NO: 116 - DNA, 19 bp, Artificial Sequence
  synthetic ssDNA
ggtgacaagcaccagaagt

SEQ ID NO: 117 - protein, 50 aa, Medicago sativa
KVCQKRSKTWSGPCLNTGNCKRQCINVEHATFGACHRQGFGFACFCYRKC

SEQ ID NO: 118 - protein, 50 aa, Medicago sativa
KLCERRSKTWSGPCLNSGHCKRQCINVEHATSGACHRQGLGIACFCKKKC

SEQ ID NO: 119 - protein, 46 aa, Lotus japonicus
GGRCSELVSRCGSSFPDCDQTCKSQHSSTNGKGFCNDNICTCYYDC

SEQ ID NO: 120 - protein, 43 aa, Lotus japonicus
RKCLAEIGPCSGQCNSNCAGRFKGATGTCNAAFGNLCICQYTC

(19) United States
(12) Patent Application Publication
(10) Pub. No.: US 2019/0194268 A1
(43) Pub. Date: Jun. 27, 2019

(54) MULTIMERIC DEFENSIN PROTEINS AND RELATED METHODS

(71) Applicant: DONALD DANFORTH PLANT SCIENCE CENTER, St. Louis, MO (US)

(72) Inventors: Dilip M Shah, St. Louis, MO (US); Siva linga sasanka Velivelli, St. Louis, MO (US); Kazi Tariqul Islam, St. Louis, MO (US)

(21) Appl. No.: 16/083,610

(22) PCT Filed: Mar. 10, 2017
(86) PCT No.: PCT/US2017/021904

§ 371 Date: Sep. 10, 2018

Related U.S. Application Data
(60) Provisional application No. 62/307,205, filed on Mar. 11, 2016.

(51) Int. Cl. C07K 14/415; C12N 15/82

(52) U.S. Cl. CPC C07K 14/415; C07K 2319/70; C07K 2319/33; C12N 15/8282

(57) ABSTRACT

Multimeric defensin proteins (MD) containing at least two defensin peptides joined by a spacer peptide that is resistant to endoproteinase cleavage are disclosed along with compositions comprising the MD proteins and transgenic or genetically edited plants or microorganisms that express the MD to inhibit growth of pathogenic fungi. Such MD proteins, compositions, plants, and microorganisms can provide for improved inhibition of fungal growth when compared to a protein containing only one of the defensin peptides found in the MD.

### CROSS-REFERENCE TO RELATED APPLICATIONS

This International Patent Application claims the benefit of U.S. provisional patent application No. 62/307,205, filed Mar. 11, 2016 and incorporated herein by reference in its entirety.

### REFERENCE TO SEQUENCE LISTING SUBMITTED ELECTRONICALLY

A sequence listing containing the file named 47004_164661_ST25.txt which is 66126 bytes (measured in MS-Windows®) and created on Mar. 9, 2017, comprises 120 sequences, is provided herewith via the USPTO's EFS system, and is incorporated herein by reference in its entirety.

### FIELD

The present disclosure relates to antifungal polypeptides that comprise at least two defensin peptides separated by a spacer peptide that is resistant to cleavage by an endoproteinase and methods for controlling pathogenic fungi with the antifungal polypeptides. The antifungal polypeptides can be applied directly to a plant, human, or animal, applied to a plant in the form of microorganisms that produce the polypeptides, or the plants can be genetically edited to produce the polypeptides. The present disclosure also relates to recombinant nucleic acids, microorganisms and plants transformed with the recombinant nucleic acids, and compositions useful in controlling pathogenic fungi.

### BACKGROUND

Protection of agriculturally important crops from pathogenic fungi is crucial in improving crop yields. Fungal infections are a particular problem in damp climates and can become a major concern during crop storage, where such infections can result in spoilage and contamination of food or feed products with fungal toxins. Unfortunately, modern growing methods, harvesting and storage systems can promote plant pathogen infections.

Control of plant pathogens is further complicated by the need to simultaneously control multiple fungi of distinct genera. For example, fungi such as Alternaria; Ascochyta; Botrytis; Cercospora; Colletotrichum; Diplodia; Erysiphe; Fusarium; Gaeumanomyces; Helminthosporium; Macrophomina; Magnaporthe; Nectria; Peronospora; Phoma; Phakopsora, Phymatotrichum; Phytophthora; Plasmopara; Podosphaera; Puccinia; Pythium; Pyrenophora; Pyricularia; Rhizoctonia; Scerotium; Sclerotinia; Septoria; Thielaviopsis; Uncinula; Venturia; and Verticillium species are all recognized plant pathogens. Consequently, resistant crop plant varieties or fungicides that control only a limited subset of fungal pathogens can fail to deliver adequate protection under conditions where multiple pathogens are present. It is further anticipated that plant pathogenic fungi can become resistant to existing fungicides and crop varieties, which can favor the introduction of new fungal control agents with distinct modes of action to combat the resistant fungi.

A group of proteins known as defensins have been shown to inhibit plant pathogens. Defensins are small cysteine-rich peptides of 45-54 amino acids that constitute an important component of the innate immunity of plants (Thomma et al., 2002; Lay and Anderson, 2005). Widely distributed in plants, defensins vary greatly in their amino acid composition. However, they all have a compact shape which is stabilized by either four or five intramolecular disulfide bonds. Plant defensins have a conserved γ-core motif comprising a conserved GXCX3-9C (where X is any amino acid) sequence (Lacerda et al., 2014). The three dimensional structure of the γ-core motif consists of two antiparallel (β-sheets, with an interpolated turn region (Ibid.). Antifungal activity of certain defensins has been correlated with the presence of positively charged amino acid residues in the γ-core motif (Spelbrink et al., Plant Physiol., 2004; Sagaram et al., 2013).

Plant defensins have been extensively studied for their role in plant defense. Some plant defensins inhibit the growth of a broad range of fungi at micromolar concentrations (Broekaert et al., 1995; Broekaert et al., 1997; da Silva Conceicao and Broekaert, 1999) and, when expressed in transgenic plants, confer strong resistance to fungal pathogens (da Silva Conceicao and Broekaert, 1999; Thomma et al., 2002; Lay and Anderson, 2005). Two small cysteine-rich proteins isolated from radish seed, Rs-AFP1 and Rs-AFP2, inhibited the growth of many pathogenic fungi when the pure protein was added to an in vitro antifungal assay medium (U.S. Pat. No. 5,538,525). Transgenic tobacco plants containing the gene encoding Rs-AFP2 protein were found to be more resistant to attack by fungi than non-transformed plants.

Antifungal defensin proteins have also been identified in Alfalfa (Medicago sativa) and shown to inhibit plant pathogens such as Fusarium and Verticillium in both in vitro tests and in transgenic plants (U.S. Pat. No. 6,916,970). Under low salt in vitro assay conditions, the Alfalfa defensin AlfAFP1 inhibited Fusarium culmorum growth by 50% at 1 ug/ml and Verticillium dahliae growth by 50% at 4 ug/ml (i.e. IC50 values of 1 ug/ml and 4 ug/ml, respectively). Expression of the AlfAFP1 protein in transgenic potato plants was also shown to confer resistance to Verticillium dahliae in both greenhouse and field tests (Gao et al., 2000). Mode-of-action analyses have also shown that AlfAFP1 (which is alternatively referred to as MsDef1, for Medicago sativa Defensin 1) induces hyper-branching of F. graminearum (Ramamoorthy et al., 2007) and can block L-type calcium channels (Spelbrink et al., 2004).

Other defensin genes have also been identified in the legume Medicago truncatula (Hanks et al., 2005). The cloned MtDef2 protein has been demonstrated through in vitro experiments to have little or no antifungal activity (Spelbrink et al., 2004). The Medicago truncatula defensin proteins MtDef4 (U.S. Pat. No. 7,825,297; incorporated herein by reference in its entirety) and MtDef5 (WO2014179260 and U.S. patent application Ser. No. 14/888,011; both incorporated herein by reference in its entirety) have antifungal activity.

Several publications have disclosed expression vectors that encode proteins having at least two defensin peptides that are liked by a peptide sequence that can be cleaved by plant endoproteinases (WO2014078900; Vasivarama and Kirti, 2013a; Francois et al.; Vasivarama and Kirti, 2013b). A MtDef5 proprotein comprising two defensin peptides separated by a small peptide linker has also been disclosed in WO2014179260 and U.S. patent application Ser. No. 14/888,011.

### SUMMARY

Antifungal polypeptides that comprise at least two defensin peptides and methods for controlling pathogenic fungi by using the antifungal polypeptides are provided herein. In certain embodiments, the defensin peptides are linked by a spacer peptide that is resistant to plant endoproteinase cleavage. The antifungal polypeptides can be applied directly to a plant, applied to a plant in the form of microorganisms that produce the polypeptides, or the plants can be transformed or genetically edited to produce the polypeptides. The present disclosure also relates to recombinant nucleic acids, microorganisms and plants transformed with the recombinant nucleic acids, and compositions useful in controlling pathogenic plant fungi.

Recombinant nucleic acid molecules comprising a polynucleotide encoding a multimeric defensin polypeptide that comprises two defensin peptides separated by a spacer peptide that is resistant to cleavage by an endoproteinase, wherein the polynucleotide is operably associated with at least one heterologous polynucleotide that promotes transcription, transcript abundance, translation, or any combination thereof are provided herein. In certain embodiments, the endoproteinase is an endogenous plant, yeast, or mammalian endoproteinase. In certain embodiments, the endoproteinase is an endogenous plant, yeast, or mammalian endoproteinase that is present in a corresponding plant, yeast, or mammalian host cell or organism in which the multimeric defensin polypeptide is expressed. In certain embodiments, a recombinant nucleic acid molecule comprising a polynucleotide encoding a multimeric defensin polypeptide that comprises two defensin peptides separated by a spacer peptide that is resistant to cleavage by a plant endoproteinase, wherein the polynucleotide is operably associated with at least one heterologous polynucleotide that promotes transcription, transcript abundance, translation, or any combination thereof is provided. In certain embodiments, a recombinant nucleic acid molecule comprising a polynucleotide encoding a multimeric defensin polypeptide that comprises two defensin peptides separated by a heterologous spacer peptide that is resistant to cleavage by a plant endoproteinase is provided. In certain embodiments of any of the aforementioned recombinant nucleic acid molecules, the polynucleotide is operably associated with at least one heterologous polynucleotide that promotes transcription, transcript abundance, translation, or a combination thereof. Recombinant polynucleotides encoding a polypeptide comprising a multimeric defensin polypeptide that has at least 70%, 75%, 80%, 85%, 90%, 95%, 98%, 99%, or 100% sequence identity across the entire length of a polypeptide selected from the group consisting of SEQ ID NO:57, SEQ ID NO:69, SEQ ID NO:73, and SEQ ID NO:112 or comprising a multimeric defensin polypeptide that has at least 92%, 95%, 98%, 99%, or 100% sequence identity across the entire length of a polypeptide of SEQ ID NO:111, wherein the polynucleotide is operably associated with at least one heterologous polynucleotide that promotes transcription, transcript abundance, translation, or a combination thereof are also provided. In certain embodiments of any of the aforementioned nucleic acid molecules, the multimeric defensin polypeptide inhibits fungal growth. In certain embodiments of any of the aforementioned nucleic acid molecules, the multimeric defensin polypeptide wherein the multimeric defensin polypeptide, when expressed by or administered to a plant, can confer resistance to infection by a plant pathogenic fungus. In certain embodiments of any of the aforementioned nucleic acid molecules, the at least one heterologous polynucleotide comprises a promoter, a 5′ untranslated region (5′ UTR), a 3′ untranslated region (3′ UTR), an intron, a polyadenylation site, or any combination thereof. In certain embodiments of any of the aforementioned nucleic acid molecules, the nucleic acid molecule further comprises a polynucleotide encoding a localization peptide that is operably associated with the multimeric defensin. In certain embodiments, the one localization peptide is an apoplast localization peptide, an endoplasmic reticulum localization peptide, a mitochondrial localization peptide, a plastid localization peptide, a vacuole localization peptide, or any combination thereof. In certain embodiments, the localization peptide is a heterologous localization peptide that can direct an operably associated polypeptide to an extracellular or sub-cellular location that is different than the extracellular or sub-cellular location of a naturally occurring polypeptide comprising one or both of the defensin peptides. In certain embodiments of any of the aforementioned nucleic acid molecules, the spacer peptide does not contain the peptide sequence of SEQ ID NO:6, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, or SEQ ID NO: 100. In certain embodiments of any of the aforementioned nucleic acid molecules, the spacer peptide lacks a plant endoproteinase recognition site. In certain embodiments of any of the aforementioned nucleic acid molecules, the spacer peptide forms a junction with each defensin peptide and each junction lacks a plant endoproteinase recognition site. In certain embodiments of any of the aforementioned nucleic acid molecules, the spacer peptide and the junctions are resistant to cleavage by a plant endoproteinase of a monocot crop plant. In certain embodiments, the monocot crop plant is selected from the group consisting of corn, barley, oat, rice, sorghum, sugarcane, pearl millet, turf grass, and wheat. In certain embodiments of any of the aforementioned nucleic acid molecules, the spacer peptide and the junctions are resistant to cleavage by a plant endoproteinase of a dicot crop plant. In certain embodiments, the dicot crop plant is selected from the group consisting of alfalfa, Brassica sp., cotton, cucurbit, potato, strawberry, sugar beet, soybean, and tomato. In certain embodiments of any of the aforementioned nucleic acid molecules, the spacer peptide is derived from a wild-type linker peptide and comprises at least one mutation that renders the mutated linker peptide resistant to cleavage by a plant endoproteinase in comparison to the corresponding wild-type linker peptide. In certain embodiments, the mutation comprises an amino acid residue insertion, deletion, substitution, or combination thereof relative to the corresponding wild-type linker peptide. In certain embodiments, the corresponding wild-type linker peptide is mutated at an amino acid residue selected from the group consisting of arginyl, lysyl, aspartyl, and glutamyl residue. In certain embodiments, the mutation eliminates a diacidic amino acid sequence, a dibasic amino acid sequence, or both in the wild-type linker peptide. In certain embodiments, the mutation eliminating a diacidic amino acid sequence, a dibasic amino acid sequence, or both in the wild-type linker peptide comprises a substitution of at least one acidic or basic amino acid residue with a glycyl, serinyl, threonyl, alaninyl, leucinyl, isoleucinyl, valinyl, prolyl, phenylalaninyl, tryptophanyl, or methionyl residue. In certain embodiments of the aforementioned nucleic acid molecules, the mutated linker peptide is resistant to cleavage by at least one of a cysteine, serine, threonine, metallo-, or aspartic plant endoproteinase. In certain embodiments of the aforementioned nucleic acid molecules, the mutated linker peptide comprises a polypeptide sequence having at least one, two, three, four, or five amino acid insertions, substitutions, or deletions in SEQ ID NO:6, SEQ ID NO:60, SEQ ID NO:70, SEQ ID NO:74, the amino acid sequence alanyl-glycyl-threonyl, or SEQ ID NO:100. In certain embodiments of any of the aforementioned nucleic acid molecules, the spacer peptide comprises a heterologous spacer peptide that comprises a SEQ ID NO:103; SEQ ID NO:104; SEQ ID NO:105, (Gly4)n sequence, a (Gly4Ser)n sequence of SEQ ID NO:93, a Ser(Gly4Ser)n sequence of SEQ ID NO:94, or combination thereof, wherein n is a positive integer equal to 1, 2, 3, 4, 5, 6, 7, 8, 9 or 10. In certain embodiments of the aforementioned nucleic acid molecules, the encoded spacer peptide is an encoded heterologous spacer peptide that is not operably associated with the encoded defensin peptides in a nucleic acid isolated from a naturally occurring host organism. In certain embodiments of the aforementioned nucleic acid molecules, the two defensin peptides are both derived from a single defensin protein, a defensin proprotein, or variant thereof. In certain embodiments of the aforementioned nucleic acid molecules, the two defensin peptides are derived from two distinct defensin proteins, two distinct defensin proproteins, or variants thereof. In certain embodiments of the aforementioned nucleic acid molecules, the multimeric defensin polypeptide can specifically bind at least one phospholipid, at least one sphingolipid, or a combination thereof. In certain embodiments, the phospholipid is selected from the group consisting of phosphatidic acid, a phosphatidylinositol monophosphate, a phosphatidylinositol bisphosphate, and combinations thereof and the sphingolipid is glucosylceramide. In certain embodiments, the phosphatidyl inositol monophosphate is phosphatidylinositol 3-phosphate, phosphatidylinositol 4-phosphate, phosphatidylinositol-5 phosphate, or any combinations thereof. In certain embodiments of the aforementioned nucleic acid molecules, binding of the multimeric defensin polypeptide to the phospholipid is increased in comparison to binding of a polypeptide consisting of a single defensin peptide of the multimeric defensin polypeptide to the phospholipid. In certain embodiments of the aforementioned nucleic acid molecules, at least one of the defensin peptides comprises an amino acid sequence at least 70%, 75%, 80%, 85%, 90%, 95%, 98%, 99%, or 100% identical to SEQ ID NO:4 , SEQ ID NO:5, SEQ ID NO:7, SEQ ID NO:38, SEQ ID NO:40, SEQ ID NO:55, SEQ ID NO:59, SEQ ID NO:61, SEQ ID NO:62, SEQ ID NO:76, SEQ ID NO:77, SEQ ID NO:79, SEQ ID NO:81, SEQ ID NO:83, SEQ ID NO:96, SEQ ID NO:97, SEQ ID NO:99, SEQ ID NO:101, SEQ ID NO:108, SEQ ID NO:109, SEQ ID NO:110, SEQ ID NO:117, SEQ ID NO:118, SEQ ID NO:119, SEQ ID NO:120, functional fragments thereof, and chimeras thereof. In certain embodiments of the aforementioned nucleic acid molecules, the two defensin peptides are the same or different and each comprises an amino acid sequence at least 70%, 75%, 80%, 85%, 90%, 95%, 98%, 99%, or 100% identical to an amino acid sequence independently selected from the group consisting of SEQ ID NO:4 , SEQ ID NO:5, SEQ ID NO:7, SEQ ID NO:38, SEQ ID NO:40, SEQ ID NO:55, SEQ ID NO:59, SEQ ID NO:61, SEQ ID NO:62, SEQ ID NO:76, SEQ ID NO:77, SEQ ID NO:79, SEQ ID NO:81, SEQ ID NO:83, SEQ ID NO:96, SEQ ID NO:97, SEQ ID NO:99, SEQ ID NO:101, SEQ ID NO:108, SEQ ID NO:109, SEQ ID NO:110, SEQ ID NO:117, SEQ ID NO:118, SEQ ID NO:119, SEQ ID NO:120, functional fragments thereof, and chimeras thereof. In certain embodiments of the aforementioned nucleic acid molecules, the defensin peptides are the same or different and each comprises an amino acid sequence at least 50%, 60%, 70%, 75%, 80%, 85%, 90%, 95%, 98%, 99%, or 100% identical to an amino acid sequence independently selected from the group consisting of SEQ ID NO:4 , SEQ ID NO:5 , SEQ ID NO:7, SEQ ID NO:38, SEQ ID NO:40, SEQ ID NO:55, SEQ ID NO:117, SEQ ID NO:118, SEQ ID NO:119, SEQ ID NO:120, functional fragments thereof, and chimeras thereof. In certain embodiments, (i) the defensin peptides are the same or different and each comprises an amino acid sequence at least 84%, 86%, 88%, 90%, 92%, 95%, 98%, 99%, or 100% identical to an amino acid sequence independently selected from the group consisting of SEQ ID NO:4 , SEQ ID NO:5, SEQ ID NO:117, SEQ ID NO:118, functional fragments thereof, and chimeras thereof. In certain embodiments, binding of the encoded polypeptide multimeric defensin polypeptide to phosphatidylserine, phosphatidylinositol 3-phosphate, phosphatidylinositol 4-phosphate, phosphatidylinositol 5-phosphate, or any combination thereof is increased in comparison to binding of a polypeptide consisting of a single defensin peptide of the multimeric defensin polypeptide to phosphatidylserine, phosphatidylinositol 3-phosphate, phosphatidylinositol 4-phosphate, phosphatidylinositol 5-phosphate, or any combination thereof. In certain embodiments of the aforementioned nucleic acid molecules, the nucleic acid is DNA, RNA, or a combination thereof. In certain embodiments of any of the aforementioned nucleic acid molecules, the multimeric defensin can comprise at least two of any of the aforementioned defensin peptides, wherein the defensin peptides are heterologous to one another. In certain embodiments of any of the aforementioned nucleic acid molecules, the permeability of a fungal plasma membrane treated with the multimeric defensin polypeptide is increased in comparison to permeability of a fungal plasma membrane treated with single defensin peptide of the multimeric defensin polypeptide. Also provided are isolated polypeptides encoded by any of the aforementioned the recombinant nucleic acid molecules.

Also provided are transformed or genetically edited host cells comprising any of the aforementioned recombinant nucleic acid molecules. In certain embodiments, the transformed or genetically edited cell is a bacterial cell, a yeast cell, or a plant cell.

Also provided are transgenic or genetically edited plants comprising any of the aforementioned recombinant nucleic acid molecules. In certain embodiments, the recombinant nucleic acid molecule confers the plant with resistance to infection by a plant pathogenic fungus in comparison to a control plant that lacks the recombinant nucleic acid molecule. In certain embodiments of any of the aforementioned plants, a plant pathogenic fungus inhibitory amount of the multimeric defensin polypeptide is an amount of at least 0.005, 0.05, 0.5, or 1 PPM in a tissue or part of the plant. In certain embodiments of any of the aforementioned plants, the plant pathogenic fungus is a Fusarium sp., Alternaria sp., Verticillium dahlia, Phytophthora sp., Colletotricum sp., Botrytis cinerea, Cercospora sp., or Puccinia sp. In certain embodiments of any of the aforementioned plants, the plant is a monocot crop plant or a dicot crop plant.

In certain embodiments, the monocot crop plant is selected from the group consisting of a corn, barley, oat, pearl millet, rice, sorghum, sugarcane, turf grass, and wheat.

In certain embodiments, the dicot crop plant is selected from the group consisting of alfalfa, a Brassica sp., cotton, cucurbit, potato, strawberry, sugar beet, soybean, and tomato. Also provided are plant parts of any of the aforementioned transgenic or genetically edited plants, wherein the plant part comprises the recombinant nucleic acid. In certain embodiments, the plant part is selected from the group consisting of a flower, leaf, root, seed, stem, and a tuber. In certain embodiments, the plant part is a non-regenerable plant part. Also provided are processed plant products of any of the aforementioned transgenic plant parts or genetically edited plant parts, wherein the processed plant product comprises the recombinant nucleic acid or a fragment thereof. In certain embodiments, the processed plant product is non-regenerable. In certain embodiments, the processed plant product is a meal or flour. In certain embodiments of any of the aforementioned plant products, the fragment comprises a recombinant polynucleotide encoding: (i) at least one junction of a defensin peptide with the heterologous spacer peptide; (ii) a junction of a heterologous localization peptide with a defensin peptide, spacer peptide, or any combination thereof; and (iii) a junction of a heterologous promoter to a defensin peptide. In certain embodiments, the processed plant product is characterized by having reduced levels of fungal toxins in comparison to processed plant products obtained from corresponding control plant crops.

Methods for obtaining a transgenic plant that is resistant to infection by a plant pathogenic fungus comprising the steps of: (i) introducing any of the aforementioned recombinant nucleic acid molecules into a plant cell, tissue, part, or whole plant; (ii) obtaining a plant cell, tissue, part, or whole plant into which the recombinant nucleic acid molecule has integrated into the plant nuclear or plastid genome; and (iii) selecting a transgenic plant obtained from the plant cell, tissue, part or whole plant of step (ii) for expression of a plant pathogenic fungus inhibitory amount of the multimeric defensin polypeptide, thereby obtaining a transgenic plant that is resistant to infection by a plant pathogenic fungus are provided. In certain embodiments, the recombinant nucleic acid molecule is introduced into the plant cell, tissue, part, or whole plant by Agrobacterium or particle-mediated transformation. In certain embodiments, the recombinant nucleic acid molecule is introduced in step (i) with a clustered regularly interspaced short palindromic repeats (CRISPR)-associated (Cas)-guide RNA or source thereof and a Cas endonuclease or source thereof, wherein the guide RNA and Cas endonuclease can form a complex that can introduce a double strand break at a target site in a nuclear genome of the plant cell, tissue, part, or whole plant.

Methods for obtaining a transgenic plant that is resistant to infection by a plant pathogenic fungus comprising the steps of: (i) providing a clustered regularly interspaced short palindromic repeats (CRISPR)-associated (Cas)-guide RNA, a Cas endonuclease, and a DNA molecule comprising a polynucleotide encoding at least one of a defensin peptide, a spacer peptide that is resistant to cleavage by a plant endoproteinase, a heterologous promoter, or a heterologous localization peptide, to a plant cell, tissue, part, or whole plant, wherein the guide RNA and Cas endonuclease can form a complex that can introduce a double strand break at a target site in a nuclear or plastid genome of the plant cell, tissue, part, or whole plant; (ii) obtaining a plant cell, tissue, part, or whole plant wherein the DNA molecule has integrated into the target site; and (iii) selecting a transgenic plant obtained from the plant cell, tissue, part or whole plant of step (ii) comprising any of the aforementioned recombinant nucleic acid molecules for expression of a plant pathogenic fungus inhibitory amount of the multimeric defensin polypeptide are provided. In certain embodiments, the polynucleotide in step (i) encodes a defensin peptide that is operably associated with a spacer peptide.

Methods for obtaining a genetically edited plant expressing a multimeric defensin comprising an amino-terminal first defensin peptide, a mutagenized linker peptide, and a carboxy-terminal second defensin peptide comprising the steps of: (i) introducing a clustered regularly interspaced short palindromic repeats (CRISPR)-associated (Cas)-guide RNA, a Cas endonuclease, and a mutagenizing polynucleotide capable of converting an endogenous wild-type genomic DNA encoding a linker peptide that is susceptible to cleavage by a plant endoproteinase to a mutagenized genomic DNA encoding a spacer peptide that is resistant to cleavage by a plant endoproteinase, into a wild-type plant cell, tissue, part, or whole plant; (ii) obtaining a plant cell, tissue, part, or whole plant wherein the nuclear genome has been mutagenized; and (iii) selecting a genetically edited plant obtained from the plant cell, tissue, part or whole plant of step (iii) for expression of a multimeric defensin comprising the first defensin peptide, the mutagenized linker peptide, and the second defensin peptide are provided. In certain embodiments, the genetically edited plant exhibits improved resistant to infection by a plant pathogenic fungus when compared to a control plant having the wild-type linker peptide domain. In certain embodiments, a wild-type linker peptide comprising an amino acid sequence that is diacidic, dibasic, or a combination thereof is converted to an amino acid sequence that lacks the diacidic, dibasic, or both the diacidic and dibasic amino acid sequences. Genetically edited plant made by any of the aforementioned methods or progeny thereof that comprise the mutagenized genomic DNA encoding the spacer peptide that is resistant to cleavage by a plant endoproteinase are also provided. Genetically edited seed obtained from any of the aforementioned genetically edited plants are also provided, where the seed comprise the mutagenized genomic DNA encoding the spacer peptide that is resistant to cleavage by a plant endoproteinase.

Also provided are methods for producing transgenic or genetically edited plant seed that provide plants resistant to infection by a plant pathogenic fungus that comprises the steps of: (i) selfing or crossing any of the aforementioned transgenic or genetically edited plants; and (ii) harvesting seed that comprises the recombinant nucleic acid molecule of the transgenic or genetically edited plant from the self or cross, thereby producing transgenic or genetically edited plant seed that provide plants resistant to infection by a plant pathogenic fungus. In certain embodiments, the transgenic plant or genetically edited plant is used as a pollen donor in the cross and the seed are harvested from a pollen recipient.

A method for reducing crop damage by a plant pathogenic fungus comprising the steps of: (i) placing seeds or cuttings any of the aforementioned transgenic or genetically edited plants in a field where control plants are susceptible to infection by at least one plant pathogenic fungus; and (ii) cultivating a crop of plants from the seeds or cuttings, thereby reducing crop damage by the plant pathogenic fungus. In certain embodiments, the method further comprises the step of harvesting seed, fruit, leaves, tubers, stems, roots, or any combination thereof from the crop. In certain embodiments, the seed, fruit, leaves, tubers, stems, roots, or any combination thereof have reduced levels of fungal toxins in comparison to seed, fruit, leaves, tubers, stems, roots, or any combination thereof obtained from corresponding control plant crops. In certain embodiments of the aforementioned methods the plant pathogenic fungus is a Fusarium sp., Alternaria sp., Verticillium dahlia, Phytophthora sp., Colletotricum sp., Botrytis cinerea, Cercospora sp., or Puccinia sp.

Polypeptides encoded by any of the aforementioned recombinant nucleic acid molecules are also provided.

Methods of producing a polypeptide encoded by any of the aforementioned recombinant nucleic acid molecules, comprising culturing the any of the aforementioned host cells under conditions in which the polypeptide encoded by the polynucleotide is expressed, and recovering the polypeptide are provided.

Compositions comprising any of the aforementioned polypeptides and a carrier are also provided

Methods for controlling fungal infection comprising the step of applying an effective amount of the aforementioned polypeptides or compositions to a subject in need thereof, a plant, a plant part, or a processed plant product are also provided. In certain embodiments, the fungal infection is caused by the plant pathogens Fusarium sp., Alternaria sp., Verticillium dahlia, Phytophthora sp., Colletotricum sp., Botrytis cinerea, Cercospora sp., or Puccinia sp. or by the human and animal fungal pathogens Aspergillus spp., Fusarium spp., Candida spp., Histoplasma capsulatum, Paracoccidiodes brasiliensis, Sporothrix shenkii, Blastomyces dermatitidis, Coccidioides spp., Geomyces destructans, Trichophyton spp. or Malassezia spp.

Use of any of the aforementioned recombinant nucleic acid molecules, transformed host cells, transgenic or genetically edited plants, transgenic or genetically edited plant parts, processed plant products, polypeptides, transgenic or genetically edited seed, or compositions to inhibit growth of a susceptible fungal species is also provided. In certain embodiments of any of the aforementioned uses, the susceptible fungal species is a Fusarium sp., Alternaria sp., Verticillium dahlia, Phytophthora sp., Colletotricum sp., Botrytis cinerea, Cercospora sp., or Puccinia sp. or is a human and animal fungal pathogen that is an Aspergillus sp., Fusarium sp., Candida sp., Histoplasma capsulatum, Paracoccidiodes brasiliensis, Sporothrix shenkii, Blastomyces dermatitidis, Coccidioides sp., Geomyces destructans, Trichophyton sp. and Malassezia sp.

### BRIEF DESCRIPTION OF THE FIGURES

FIG. 1 shows the complete nucleotide sequence of the MtDef5FL gene (SEQ ID NO:1).

FIG. 2A, B, C shows the coding and predicted amino acid sequences of three MtDef5 proproteins, each containing an apoplast targeting peptide (bold and underlined), two defensin peptides (in upper case), and a peptide separating the defensin peptides (underlined). FIG. 2A shows the Medtr8g012775.1 (MtDef5FL1) nucleotide (SEQ ID NO:1) and amino acid (SEQ ID NO:2) sequences, FIG. 2B shows the Medtr8g012775.2 (MtDef5FL2) nucleotide (SEQ ID NO:67) and amino acid (SEQ ID NO:68) sequences, and FIG. 2C shows the Medtr8g012775.3 (MtDef5FL3) nucleotide (SEQ ID NO:71) and amino acid (SEQ ID NO:72) sequences.

FIG. 3 shows the pPIC9 MtDef5FL vector map.

FIG. 4A,B Panel A shows Coumassie Blue-stained gel showing the purified MtDef5FL1 and MtDef5 defensin peptide A. The sizes of the purified peptides are larger than the actual sizes of the peptides on this gel system. Panel B is a Western blot analysis showing that MtDef5A-specific polyclonal antibody cross-reacting only with the 11.9 kDa MtDef5FL1 protein and 5.6 kDa MtDef5 defensin peptide A.

FIG. 5A, B shows in vitro antifungal activity of MtDef5FL. (A) Quantitative assessment of the inhibition of fungal growth of F. graminearum, F. virguliforme and N. crassa at different concentrations of MtDef5FL. Values are means of three replications. Error bars indicates standard deviations. (B) Images showing the inhibition of F. graminearum, F. virguliforme and N. crassa conidial germination and hyphal growth at different concentration of MtDef5FL. Bar=20

FIG. 6 shows plasma membrane permeabilization by MtDef5FL of fungal hyphae. Fluorescence images of SG binding to the nuclei of F. graminearum, F. virguliforme and N. crassa, following treatment with 0.046 μM of MtDef5FL+0.5 μM SG. Bar=20 μm.

FIG. 7 shows the Leersia perrieri defensin LpDef1 proprotein (SEQ ID NO:56) containing an apoplast targeting peptide (bold and underlined), two (2) defensin peptides (in upper case), and a linker peptides separating the defensin peptides- (underlined). A C-terminal extension (lower case) follows the second defensin peptide in the proprotein.

FIG. 8 shows a CLUSTAL O(1.2.1) multiple sequence alignment (Uniprot) of the defensin peptides of ZmESR-6 (SEQ ID NO:62) with the Leersia perrieri defensin LpDef1 defensin A (SEQ ID NO:59) and defensin B (SEQ ID NO:61).

FIG. 9 shows a comparison of the phospholipid binding activities of the MtDef5 defensin A protein (SEQ ID NO:4) and the MtDef5FL1 protein (SEQ ID NO:7).

FIG. 10 shows an alignment of various plant defensin peptides (SEQ ID NO:8 to SEQ ID NO:55) to the MtDef5 defensin A (SEQ ID NO:4) and defensin B (SEQ ID NO:5).

FIG. 11 shows a map of the Ds-Red-MtDef5FL vector.

FIG. 12 shows a Western blot analysis of the total protein extracted from the leaf tissue of the transgenicArabidopsisline transformed with the Ds-Red-MtDef5FL vector. The expression of the predicted 11.9 kDa MtDef5FL1 protein is evident in this line.

FIG. 13 shows the deduced amino acid sequences of MtDef5FL1 (SEQ ID NO: 2). The 29-amino acid signal peptide sequence is shown in lower case, italics and underlined. The mature MtDef5FL1 protein contains two defensin peptides (A and B), each 50 amino acids in length, connected by a 7-amino acid linker shown in lower case and underlined. Defensin peptides A and B are underlined. The two γ-core motifs of MtDef5FL1 defensin peptides A and 5 peptides are shown in italics and underlined.

FIG. 14A, B shows in vitro antifungal activity of MtDef5FL1. (A) Quantitative assessment of the inhibition of fungal growth of F. graminearum and N. crassa at different concentrations of MtDef5FL1. Values are means of three replications. Error bars indicates standard deviations. (B) The growth of both fungi is completely inhibited at a concentration of 0.75 μM. Bar=20 μm.

FIG. 15 shows activity of MtDef5FL1 against various fungal pathogens.

FIG. 16 shows permeabilization of MtDef5FL1 into fungal hyphae. Fluorescence images of SG binding to the nuclei of F. graminearum and N. crassa, following treatment with 0.75 μM of MtDef5FL1+0.5 μM SG. Bar=20 μm.

FIG. 17 shows that MtDef5FL1 is internalized into F. graminearum. MtDef5FL1 has a diffuse localization within the cytoplasm of F. graminearum. DyLight550 labeled MtDef5 is internalized and diffusely localized within the cytoplasm of conidia and germlings of F. graminearum (upper and lower panels, respectively). Image were taken 3 hr of treatment. Bar=5 μm.

FIG. 18A, B shows the binding of MtDef5FL1 with phospholipids (A) PIP strip showing that MtDef5FL1 strongly binds to phosphatidylinositol monophosphates (B) PIP array displays the relative PIP binding of MtDef5FL1.

FIG. 19 shows that MtDef5FL1 forms higher order oligomers in presence of PIPs as revealed by a protein cross-linking assay.

FIG. 20 shows that MtDef5FL1 forms nanonet structures in presence of PIP. MtDef5FL1+cross linker (BS3) alone or PIP+crosslinker (BS3) do not form nanonet structures.

FIG. 21A, B, C shows: A. Sequences of the core variants with respect to the MtDef5FL1 defensin peptide A (SEQ ID NO:4) and MtDef5FL1 defensin peptide B (SEQ ID NO:5); B. In vitro antifungal activity of MtDef5FL1 γ-core motif variants, C. Membrane permeabilization by MtDef5FL1 γ-core motif variants. MtDef5FL1_V3 induces membrane permeabilization faster than the wild-type MtDef5FL1 as measured by the uptake of SYTOX green.

FIG. 22 shows the interaction of MtDef5FL1 and it variants with phospholipids. PIP strip shows that MtDef5FL1 and it variants strongly binds strongly to phosphatidylinositol monophosphates PI3P, PI4P and PI5P.

FIG. 23 shows that MtDef5FL1 γ-core motif variants exhibit much reduced oligomerization in presence of PI(3)Ps.

FIG. 24A, B shows MtDef5FL1 expression analysis of transgenic Arabidopsis lines (Defy-5, Def5-10, Def5-14) by RT-qPCR. Relative expression was normalized to Ubiq10 gene. Error bars represent SE of three different biological replicates. The Wild-type Col-0 and Null line did not show any transcript accumulation. b) Western blot analysis of the MtDef5 in transgenicArabidopsislines. Transgenic lines showed the expression of ˜24 kD protein and not the expected 11.9 kD protein. This ˜24 kD protein is not present in the wild-type and null lines.

FIG. 25 shows that transgenic lines Def5-5 (SV2), Def5-10 (SV3) and Def5-14 (SV4) exhibit strong resistance to the downy mildew pathogen Hyaloperonospora arabidopsidis Noco2 isolate.

### DETAILED DESCRIPTION

### Definitions

As used herein, a polynucleotide is said to be “endogenous” to a given cell when it is found in a naturally occurring form and genomic location in the cell.

The phrases “antifungal polypeptide” or “antifungal protein” as used herein refer to polypeptides or proteins which exhibit any one or more of the following characteristics of inhibiting the growth of fungal cells, killing fungal cells, disrupting or retarding stages of the fungal life cycle such as spore germination, sporulation, or mating, and/or disrupting fungal cell infection, penetration or spread within a plant or other susceptible subject.

As used herein, the phrase “consensus sequence” refers to an amino acid, DNA or RNA sequence created by aligning three or more homologous sequences and deriving a new sequence having the most prevalent amino acid, nucleic acid or ribonucleic acid residue of the homologous sequences at each position in the created sequence.

The phrases “combating fungal damage”, “combating or controlling fungal damage” or “controlling fungal damage” as used herein refer to reduction in damage to a crop plant or crop plant product due to infection by a fungal pathogen. More generally, these phrases refer to reduction in the adverse effects caused by the presence of a pathogenic fungus in the crop plant. Adverse effects of fungal growth are understood to include any type of plant tissue damage or necrosis, any type of plant yield reduction, any reduction in the value of the crop plant product, and/or production of undesirable fungal metabolites or fungal growth by-products including but not limited to mycotoxins.

The phrase “defensin peptide” is used herein to refer to a peptide comprising a conserved γ-core motif comprising a conserved GXCX3-9C sequence, where X is any amino acid residue. Defensin peptides include, but are not limited to, proteins that are antifungal, that can bind phospholipids, that can bind sphingolipids, or that exhibit any combination of those properties. A defensin peptide can be naturally occurring or non-naturally occurring (e.g., synthetic and/or chimeric).

The term “endoproteinase” is used herein to refer to a peptidase capable of cleaving a peptide bond between two internal amino acid residues in a polypeptide sequence. Endoproteinases can also be referred to as “endoproteases” or “endopeptidases.” The proteolytic activity of an endoproteinase, endoprotease, or endopeptidase is thus different that the proteolytic activity of an “exopeptidase” which cleaves peptide bonds of terminal amino acid residues in a polypeptide.

The phrase “genetically edited plant” is used herein to refer to a plant comprising one or more nucleotide insertions, deletions, substitutions, or any combination thereof in the genomic DNA of the plant where any insertion is of less than 10 nucleotides in length. Such genetically edited plants can be constructed by techniques including, but not limited to, CRISPR/Cas endonuclease-mediated editing, meganuclease-mediated editing, engineered zinc finger endonuclease-mediated editing, and the like.

The term “heterologous”, as used herein in the context of a second polynucleotide that is operably linked to a first polynucleotide, refers to: (i) a second polynucleotide that is derived from a source distinct from the source of the first polynucleotide; (ii) a second polynucleotide derived the same source as the first polynucleotide, where the first, second, or both polynucleotide sequence(s) is/are modified from its/their original form; (iii) a second polynucleotide arranged in an order and/or orientation or in a genomic position or environment with respect to the first polynucleotide that is different than the order and/or orientation in or genomic position or environment of the first and second polynucleotides in a naturally occurring cell; or (iv) the second polynucleotide does not occur in a naturally occurring cell that contains the first polynucleotide. Heterologous polynucleotides include, but are not limited to, polynucleotides that promote transcription (e.g., promoters and enhancer elements), transcript abundance (e.g., introns, 5′UTR, and 3′UTR), translation, or a combination thereof as well as polynucleotides encoding defensin peptides, spacer peptides, or localization peptides. A “heterologous” polynucleotide that promotes transcription, transcript abundance, translation, or a combination thereof as well as polynucleotides encoding defensin peptides, spacer peptides, or localization peptides can be autologous to the cell but, however, arranged in an order and/or orientation or in a genomic position or environment that is different than the order and/or orientation in or genomic position or environment in a naturally occurring cell. A polynucleotide that promotes transcription, transcript abundance, translation, or a combination thereof as well as polynucleotides encoding defensin peptides, spacer peptides, or localization can be heterologous to another polynucleotide when the polynucleotides are not operably linked to one another in a naturally occurring cell. Heterologous polypeptides include, but are not limited to, polypeptides that are not found in a cell or organism as the cell or organism occurs in nature. As such, heterologous polypeptides include, but are not limited to, polypeptides that are localized in a subcellular location, extracellular location, or expressed in a tissue that is distinct from the subcellular location, extracellular location, or tissue where the polypeptide is found in a cell or organism as it occurs in nature.

The term “homolog” as used herein refers to a gene related to a second gene by identity of either the DNA sequences or the encoded protein sequences. Genes that are homologs can be genes separated by the event of speciation (see “ortholog”). Genes that are homologs can also be genes separated by the event of genetic duplication (see “paralog”). Homologs can be from the same or a different organism and can in certain embodiments perform the same biological function in either the same or a different organism.

The phrases “inhibiting growth of a plant pathogenic fungus”, “inhibit fungal growth”, and the like as used herein refers to methods that result in any measurable decrease in fungal growth, where fungal growth includes but is not limited to any measurable decrease in the numbers and/or extent of fungal cells, spores, conidia, or mycelia. As used herein, “inhibiting growth of a plant pathogenic fungus” is also understood to include any measurable decrease in the adverse effects cause by fungal growth in a plant. Adverse effects of fungal growth in a plant include any type of plant tissue damage or necrosis, any type of plant yield reduction, any reduction in the value of the crop plant product, and/or production of undesirable fungal metabolites or fungal growth by-products including but not limited to mycotoxins. As used herein, the phrase “inhibition of fungal growth” and the like, unless otherwise specified, can include inhibition in a plant, human or animal.

As used herein, the phrase “junction sequence”, when used in the context of a multimeric defensin, refers to an amino acid sequence of about six residues where at least three (3) residues are contributed by a spacer peptide and at least three (3) residues are contributed by a defensin peptide. In certain embodiments, 3 amino acids at the N-terminus of the junction sequence are contributed by the final 3 C-terminal residues of the defensin sequence and 3 amino acids at the C-terminus of the junction sequence are contributed by the first 3 N-terminal residues of the spacer peptide sequence. In certain embodiments, 3 amino acids at the N-terminus of the junction sequence are contributed by the final 3 C-terminal residues of the spacer peptide sequence and 3 amino acids at the C-terminus of the junction sequence are contributed by the first 3 N-terminal residues of the defensin peptide sequence.

As used herein, the phrase “linker peptide” refers to any peptide that joins two defensin peptides in a protein. In certain embodiments, a linker peptide can be susceptible to cleavage by an endoproteinase. In certain alternative embodiments, a linker peptide can be a spacer peptide that is resistant to endoproteinase cleavage. One embodiment where a linker peptide can be (e.g., function as) a spacer peptide is when the linker peptide that joins two defensin peptides is localized in an extracellular or sub-cellular location that is deficient in endogenous endoproteinases that can cleave that linker peptide. One embodiment where a linker peptide can be (e.g., function as) a spacer peptide is when the linker peptide is joined to one or more heterologous defensin peptides that render the linker peptide resistant to endoproteinase cleavage. Another embodiment where a linker peptide can be (e.g., function as) a spacer peptide is when the linker peptide is joined to defensin peptide(s) via a heterologous junction sequence or sequences that render the linker peptide resistant to endoproteinase cleavage. A linker peptide can be naturally occurring or non-naturally occurring (e.g., synthetic).

As used herein, the phrase “linker peptide that is susceptible to cleavage by a endoproteinase”, when used in the context of a linker peptide sequence that joins two defensin peptides in a single encoded polypeptide, refers to a linker peptide sequence that permits less than 50% of defensin containing protein in a transgenic or genetically edited organism or cell, an extracellular space of the organism or cell, a sub-cellular location of the organism or cell, or any combination thereof to accumulate as a polypeptide comprising the linker peptide and both defensin peptides that are covalently linked thereto. The phrase “linker peptide that is susceptible to cleavage by a plant endoproteinase”, when used in the context of a linker peptide sequence that joins two defensin peptides in a single encoded polypeptide, refers to a linker peptide sequence that permits less than 50% of defensin containing protein in a transgenic or genetically edited plant or cell, an extracellular space of the plant or cell, a sub-cellular location of the plant or cell, or any combination thereof to accumulate as a polypeptide comprising the linker peptide and both defensin peptides that are covalently linked thereto. In certain embodiments, the endoproteinase is an endogenous plant, yeast, or mammalian endoproteinase.

The phrase “operably linked” as used herein refers to the joining of nucleic acid or amino acid sequences such that one sequence can provide a function to a linked sequence. In the context of a promoter, “operably linked” means that the promoter is connected to a sequence of interest such that the transcription of that sequence of interest is controlled and regulated by that promoter. When the sequence of interest encodes a protein that is to be expressed, “operably linked” means that the promoter is linked to the sequence in such a way that the resulting transcript will be efficiently translated. If the linkage of the promoter to the coding sequence is a transcriptional fusion that is to be expressed, the linkage is made so that the first translational initiation codon in the resulting transcript is the initiation codon of the coding sequence. Alternatively, if the linkage of the promoter to the coding sequence is a translational fusion and the encoded protein is to be expressed, the linkage is made so that the first translational initiation codon contained in the 5 untranslated sequence associated with the promoter and the coding sequence is linked such that the resulting translation product is in frame with the translational open reading frame that encodes the protein. Nucleic acid sequences that can be operably linked include, but are not limited to, sequences that provide gene expression functions (e.g., gene expression elements such as promoters, 5′ untranslated regions, introns, protein coding regions, 3 ‘ untranslated regions, polyadenylation sites, and/or transcriptional terminators), sequences that provide DNA transfer and/or integration functions (e.g., T-DNA border sequences, site specific recombinase recognition sites, integrase recognition sites), sequences that provide for selective functions (e.g., antibiotic resistance markers, biosynthetic genes), sequences that provide scoreable marker functions (e.g., reporter genes), sequences that facilitate in vitro or in vivo manipulations of the sequences (e.g., polylinker sequences, site specific recombination sequences) and sequences that provide replication functions (e.g., bacterial origins of replication, autonomous replication sequences, centromeric sequences). In the context of an amino acid sequence encoding a localization, spacer, or other peptide, “operably linked” means that the peptide is connected to the polyprotein sequence(s) of interest such that it provides a function. Functions of a localization peptide include, but are not limited to, localization of a polyprotein of interest (e.g., an MD) to an extracellular space or subcellular compartment. Functions of a spacer peptide include, but are not limited to, linkage of two polyproteins of interest (e.g., two defensin peptides) such that the polypeptides will be expressed as a single polypeptide (e.g., an MD).

The phrase “percent identity” as used herein refers to the number of elements (i.e., amino acids or nucleotides) in a sequence that are identical or similar within a defined length of two optimally aligned DNA, RNA or protein segments. In one embodiment, the “percent identity” can be calculated by dividing the number of identical elements by the total number of elements in the defined length of the aligned segments and multiplying by 100. When percentage of identity is used in reference to proteins it is understood that certain amino acid residues that are not identical but are nonetheless conservative or similar amino acid substitutions that reflect substitutions of amino acid residues with similar chemical properties (e.g., acidic or basic, hydrophobic, hydrophilic, hydrogen bond donor or acceptor residues). In certain embodiments, such substitutions do not change the functional properties of the molecule. Consequently, the percent identity of protein sequences can be increased to account for conservative (e.g., similar) substitutions. In certain embodiments, percent identity can be achieved by using public or proprietary algorithms. In certain embodiments, percent identity or similarity can be determined using pairwise alignment methods, e.g., BLAST, BLAST-2, ALIGN, or ALIGN-2 or multiple sequence alignment methods such as Megalign (DNASTAR), ClustalW or T-Coffee software. Appropriate scoring functions, e.g., gap penalties or scoring matrices for measuring alignment, including any algorithms needed to achieve optimal alignment quality over the full-length of the sequences being compared. In addition, sequence alignment can be achieved using structural alignment methods (e.g., methods using secondary or tertiary structure information to align two or more sequences), or hybrid methods combining sequence, structural, and phylogenetic information to identify and optimally align candidate protein sequences. The NCBI Basic Local Alignment Search Tool (BLAST) (Altschul et al., J. Mol. Biol. (1990) 215:403) is available from several sources, including the National Center for Biotechnology Information (NCBI, Bethesda, Md.) and on the internet, for use in connection with the sequence analysis programs blastp, blastn, blastx, tblastn and tblastx.

As used herein, the phrase “resistant to cleavage by an endoproteinase”, when used in the context of a spacer peptide sequence that joins two defensin peptides in a single encoded polypeptide, refers to a spacer peptide sequence that permits more than 50%, 60%, 70%, 80%, 90%, or 95% of the defensin peptide containing protein in a transgenic or genetically edited organism, cell, extracellular space of the organism or cell, sub-cellular location of the organism or cell, or any combination thereof to accumulate as a multimeric defensin protein that comprises the spacer peptide and both defensin peptides that are covalently linked thereto. The phrase “resistant to cleavage by a plant endoproteinase”, when used in the context of a spacer peptide sequence that joins two defensin peptides in a single encoded polypeptide, refers to a spacer peptide sequence that permits more than 50%, 60%, 70%, 80%, 90%, or 95% of the defensin peptide containing protein in a transgenic or genetically edited plant or plant cell, an extracellular space of the plant or cell, a sub-cellular location of the plant or cell, or any combination thereof to accumulate as a multimeric defensin protein that comprises the spacer peptide and both defensin peptides that are covalently linked thereto.

As used herein, the phrase “spacer peptide” refers to any peptide that joins two defensin peptides in a protein that is resistant to cleavage by an endoproteinase. In certain embodiments, the endoproteinase is an endogenous plant, yeast, or mammalian endoproteinase. A spacer peptide can be naturally occurring or non-naturally occurring (e.g., synthetic).

The terms “susceptible fungus (or fungi)”, “susceptible fungal infection”, and the like refer to fungi that infect plants, or human or animal patients or subjects, or fungal infections thereof, that are subjection to inhibition of fungal growth by the MD molecules disclosed herein.

The phrase “transgenic organism” refers to an organism or progeny thereof wherein the organism's or progeny organism's DNA of the nuclear or organellar genome contains an inserted exogenous DNA molecule of 10 or more nucleotides in length. The phrase “transgenic plant” refers to a plant or progeny thereof wherein the plant's or progeny plant's DNA of the nuclear or plastid genome contains an introduced exogenous DNA molecule of 10 or more nucleotides in length. Such introduced exogenous DNA molecules can be naturally occurring, non-naturally occurring (e.g., synthetic and/or chimeric), from a heterologous source, or from an autologous source.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

### Further Description

Antifungal polypeptides referred to as “multimeric defensins” (MD) that comprise at least two defensin peptides are provided herein. In certain embodiments, the defensin peptides in the MD are linked by a spacer peptide that is resistant to plant endoproteinase cleavage. The antifungal polypeptides can be applied directly to a plant, applied to a plant in the form of microorganisms that produce the MD, or the plants can be genetically edited to produce the MD. The present disclosure also relates to recombinant nucleic acids, microorganisms and plants transformed with the recombinant nucleic acids, and compositions useful in controlling pathogenic fungi including, but not limited to, plant pathogenic fungi. In certain embodiments, the MD proteins can provide for improved inhibition of fungal growth when compared to a protein containing only one of the defensin peptides found in the MD.

Spacer peptide domains that can be used to join defensin peptides in the MD can be obtained from a variety of sources. In certain embodiments, the spacer peptides are obtained from linker peptides that join defensin proteins. Such linker peptides can be used as is or can be mutagenized. Examples of linker peptides that can be used as is or in a mutagenized form include, but are not limited to, (i) MtDef5FL linker peptide domains comprising SEQ ID NO:70 and SEQ ID NO:74; (ii) the LfDef1 linker peptide domains comprising SEQ ID NO:60, SEQ ID NO:62, and SEQ ID NO:64; (iii) A. thaliana At5g38330 linker peptide domains comprising the peptide sequence N-alanyl-glycyl-threonyl-C (“AGT”); and (iv) A. thaliana At4g30070 linker peptide domain comprising SEQ ID NO:100. Other examples of linker peptide domains that can be used in mutagenized form include, but are not limited to, a MtDef5FL linker peptide domains comprising SEQ ID NO:6 and SEQ ID NO:60. Mutagenesis of any of the aforementioned linker peptides can entail the insertion, deletion, or substitution of at least one, two, three, four, five, six, or seven amino acid residues in the linker peptide sequence that render the mutagenized linker peptide resistant to cleavage by a plant endoproteinase. In certain embodiments, diacidic (aspartyl residues, glutamyl residues, and any combination thereof), dibasic (arginyl residues, lysyl residues, and any combination thereof), or combinations of diacidic and dibasic residues located in a linker peptide sequence are targeted for mutagenesis. Disruption of such diacidic and dibasic residues in the linker peptide sequence by mutagenesis can provide mutagenized linker peptide sequences that are resistant to cleavage by a plant endoproteinase. In certain embodiments, the dibasic residues in SEQ ID NO:6 (i.e., the two lysyl residues) are mutagenized to provide a spacer peptide. In certain embodiments of any of the aforementioned mutageneses, at least one residue in a dibasic or diacidic peptide sequence in the peptide linker is substituted with a glycyl, serinyl, threonyl, alaninyl, leucinyl, isoleucinyl, valinyl, prolyl, phenylalaninyl, tryptophanyl, or methionyl residue. In certain embodiments, the linker peptide or mutated linker peptide is resistant to cleavage by at least one of a cysteine, serine, threonine, metallo-, or aspartic plant endoproteinase. A variety of amino sequences have been identified as plant endoproteinase substrates (Tsiatsiani et al., 2012) and can be mutagenized when present in a linker peptide or other sequence that is used to obtain a spacer peptide for an MD. Resistant to cleavage by at least one of a cysteine, serine, threonine, metallo-, or aspartic plant endoproteinase can be assessed by determining if an MD that incorporates a spacer peptide, a linker peptide, or a mutagenized linker peptide between defensin peptides is cleaved by the plant endoproteinase in either: (i) an in vitro assay where the MD is incubated with a plant endoproteinase under conditions where the endoproteinase is active and determining if the MD is cleaved; or (ii) expressing the MD in a transformed plant cell, tissue, or whole plant with a plant expression vector and determining if the MD is cleaved. Examples of non-limiting methods for determining if a MD is cleaved by an endoproteinase include immunoblot analysis of proteins that have been resolved by SDS-polyacrylamide gel electrophoresis or other techniques such as MALDI-TOF mass spectroscopy and the like. Spacer peptides for use in MD that comprise mutagenized linker peptide sequences having at least 60%, 70%, 80%, 85%, 90%, 95%, 96%, 97%, 98%, or 99% sequence identity to SEQ ID NO:6, SEQ ID NO:60, SEQ ID NO:62, SEQ ID NO:64, SEQ ID NO:70, SEQ ID NO:74, the sequence “AGT”, or SEQ ID NO:100 are provided herein.

Spacer peptides for use in the MD can also be obtained from multimeric- or multi-domain proteins that do not contain defensin peptides. Such peptide linker sequences that join defensin peptides in multimeric or multi-domain proteins have been disclosed (Argos, 1990; George R A, Heringa (2002). Examples of suitable peptide sequences from multimeric or multi-domain proteins that can be used as spacer domains include, but are not limited, immunoglobulin hinge regions from immunoglobulins, a linker between the lipoyl and E3 binding domain in pyruvate dehydrogenase (Turner et al., 1993), a linker between the central and C-terminal domains in cysteine proteinase (P9; Mottram et al., 1989), and functional variants thereof.

Spacer peptides for use in the MD can also be wholly or partially synthetic peptide sequences. Such synthetic spacer peptides are designed to provide for a flexible linkage between the defensin peptides and to be resistant to cleavage by endogenous plant endoproteinases. In certain embodiments, the length of the synthetic spacer peptide can be between about 3, 4, 8, 10, 12, or 16 and about 20, 24, 28, 30, 40, or 50 amino acid residues in length. In certain embodiments, the synthetic spacer peptide can comprise a glycine-rich or glycine/serine containing peptide sequence. Such sequences can include, but are not limited to, a (Gly4)n sequence, a (Gly4Ser)n sequence of SEQ ID NO:93, a Ser(Gly4Ser)n sequence of SEQ ID NO:94, combinations thereof, and variants thereof, wherein n is a positive integer equal to 1, 2, 3, 4, 5, 6, 7, 8, 9 or 10. In certain embodiments, such glycine-rich or glycine/serine containing synthetic peptide sequences can also contain threonyl and/or alanyl residues for flexibility as well as polar lysyl and/or glutamyl residues. Additional synthetic linker sequences that can be used as spacer peptides include, but are not limited to, SEQ ID NO:103, SEQ ID NO:104, SEQ ID NO:105, combinations thereof, and variants thereof. Such variants of synthetic linker sequences include, but are not limited to, insertions, deletions, and substitutions of amino acid residues. Variants of any of the aforementioned synthetic peptide spacers also include, but are not limited, to insertions and/or substitutions of one or more residues that frequently occur in peptides that join domains in proteins such as prolyl, arginyl, phenylalanyl, threonyl, glutamyl, glutaminyl, and combinations thereof. In certain embodiments, such glycine-rich, glycine/serine containing peptide sequence, or other synthetic peptide spacer sequence can be used to mutagenize a linker peptide sequence. In certain embodiments, mutagenesis of a linker peptide sequence by insertion and/or substitution of a glycine-rich or glycine/serine containing peptide sequence can be used to disrupt a peptide sequence recognized by a plant endoproteinase such as a set of diacidic and/or dibasic residues or a site that is cleaved by a cysteine, serine, threonine, metallo-, or aspartic plant endoproteinase. Insertion and/or substitution of a glycine-rich, a glycine/serine peptide sequence, or other synthetic spacer peptide into a linker peptide sequence including, but not limited to, SEQ ID NO:6, SEQ ID NO:60, SEQ ID NO:62, SEQ ID NO:64, SEQ ID NO:70, SEQ ID NO:74, the sequence “AGT”, or SEQ ID NO:100 is also provided herein. In certain embodiments, the spacer peptide can comprise a sequence that contains prolyl, arginyl, phenylalanyl, threonyl, glutamyl, glutaminyl residues, and combinations thereof. The composition and design of peptides suitable for flexible linkage of protein domains described in the literature (Chen et al., 2013) can be adapted for use as spacer peptides in the MD provided herein.

Since the defensin peptides are joined to one another in the MD, the spacer peptide sequences and the junction sequences formed by joining either the amino- or carboxy-terminus of a defensin to a spacer peptide are in certain embodiments also designed or engineered to be free of amino acid sequences that are susceptible to cleavage by plant endoproteinases. In designing MD for expression in plant hosts, the spacer peptide and junction sequences will typically lack diacidic (aspartyl residues, glutamyl residues, and any combination thereof), dibasic (arginyl residues, lysyl residues, and any combination thereof), or combinations of diacidic and dibasic residues in certain embodiments provided herein. Spacer peptide and junction sequences will typically be resistant to cleavage by at least one of a cysteine, serine, threonine, metallo-, or aspartic plant endoproteinase in certain embodiments provided herein. Amino acid sequences identified as plant endoproteinase substrates (Tsiatsiani et al., 2012) will also typically be absent from spacer peptide and junction sequences in certain embodiments provided herein.

In certain embodiments, the MD provided herein can comprise a spacer peptide or junction sequence that is susceptible to cleavage by a plant endoproteinase when the MD is expressed in a plant, plant cell, yeast cell, or mammalian cell manner that that will prevent such cleavage. In one such embodiment, the MD that comprises a spacer peptide or junction sequence that is susceptible to cleavage by a plant endoproteinase is targeted to an extracellular or sub-cellular compartment where activity of that plant endoproteinase reduced or absent. In certain embodiments, an MD that comprises a spacer peptide or junction sequence that is susceptible to cleavage by a vacuolar plant endoproteinase is targeted to either the apoplast, plastids, mitochondria, or endoplasmic reticulum by operable linkage of suitable localization peptides to that MD and/or by removal of any vacuolar localization signal that could have been associated with a given MD. In certain embodiments, an MD that comprises a spacer peptide or junction sequence that is susceptible to cleavage by a plastidic plant endoproteinase is targeted to either the apoplast, mitochondria, endoplasmic reticulum, or vacuole by operable linkage of suitable localization peptides to that MD and/or by removal of any plastid localization signal that could have been associated with a given MD. In certain embodiments, an MD that comprises a spacer peptide or junction sequence that is susceptible to cleavage by an apoplastic plant endoproteinase is targeted to either mitochondria, plastids, endoplasmic reticulum, or vacuole by operable linkage of suitable localization peptides to that MD. In certain embodiments, an MD that comprises a spacer peptide or junction sequence that is susceptible to cleavage by a mitochondrial plant endoproteinase is targeted to an apoplastic space, plastids, endoplasmic reticulum, or vacuole by operable linkage of suitable localization peptides to that MD. Also provided herein are embodiments where an MD that comprises one or more spacer peptides that are resistant to cleavage by a plant endoproteinase is targeted to the apoplast, plastids, mitochondria, vacuole, or endoplasmic reticulum.

A variety of different defensin peptides can be used in the MD provided herein. In certain embodiments, the defensin peptides in the MD will be identical or related to one another such that the two peptides have at least 60%, 70%, 80%, 85%, 90%, 95%, 98%, or 99% sequence identity. In certain embodiments, the defensin peptides will be distinct and have less than 60% identity to one another. In any of the aforementioned embodiments, the defensin peptide(s) can comprise an amino acid sequence that is at least 70%, 75%, 80%, 85%, 90%, 95%, 98%, 99%, or 100% identical to SEQ ID NO:4, SEQ ID NO:5, SEQ ID NO:7, SEQ ID NO:38, SEQ ID NO:40, SEQ ID NO:55, SEQ ID NO:59, SEQ ID NO:61, SEQ ID NO:62, SEQ ID NO:76, SEQ ID NO:77, SEQ ID NO:79, SEQ ID NO:81, SEQ ID NO:83, SEQ ID NO:96, SEQ ID NO:97, SEQ ID NO:99, SEQ ID NO:101, SEQ ID NO:108, SEQ ID NO:109, SEQ ID NO:110, SEQ ID NO:117, SEQ ID NO:118, SEQ ID NO:119, SEQ ID NO:120, functional fragments thereof, and chimeras thereof. In certain embodiments, defensin peptide variants used in the MD can comprise an amino acid sequence that is at least 70%, 75%, 80%, 85%, 90%, 95%, 98%, or 99% identical to SEQ ID NO:4 , SEQ ID NO:5, SEQ ID NO:7-SEQ ID NO:55, SEQ ID NO:59, SEQ ID NO:61, SEQ ID NO:62, SEQ ID NO:76, SEQ ID NO:77, SEQ ID NO:79, SEQ ID NO:81, SEQ ID NO:83, SEQ ID NO:96, SEQ ID NO:97, SEQ ID NO:99, SEQ ID NO:101, SEQ ID NO:108, SEQ ID NO:109, SEQ ID NO:110, SEQ ID NO:117, SEQ ID NO:118, SEQ ID NO:119, or SEQ ID NO:120. In any of the aforementioned embodiments, the variant defensin peptide(s) can also comprise an amino acid sequence that has at least one, two, three, four, five, six, or seven amino acid insertions, deletions, substitutions, or any combination thereof in a SEQ ID NO:4, SEQ ID NO:5, SEQ ID NO:7, SEQ ID NO:38, SEQ ID NO:40, SEQ ID NO:55, SEQ ID NO:59, SEQ ID NO:61, SEQ ID NO:62, SEQ ID NO:76, SEQ ID NO:77, SEQ ID NO:79, SEQ ID NO:81, SEQ ID NO:83, SEQ ID NO:96, SEQ ID NO:97, SEQ ID NO:99, SEQ ID NO:101, SEQ ID NO:108, SEQ ID NO:109, SEQ ID NO:110, SEQ ID NO:117, SEQ ID NO:118, SEQ ID NO:119, or SEQ ID NO:120 defensin peptide sequence. In certain embodiments, the MD can comprise at least two of any of the aforementioned defensin peptides, wherein the defensin peptides are heterologous to one another.

In certain embodiments, one or more amino acids in any of the aforementioned or other variant defensin peptide sequences are substituted with another amino acid(s), the charge and polarity of which is similar to that of the original amino acid, i.e., a conservative amino acid substitution. Substitutes for an amino acid within the defensin peptide sequence can be selected from other members of the class to which the originally occurring amino acid belongs. Amino acids can be divided into the following four groups: (1) acidic amino acids; (2) basic amino acids; (3) neutral polar amino acids; and (4) neutral non-polar amino acids. Representative amino acids within these various groups include, but are not limited to: (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, cystine, tyrosine, asparagine, and glutamine; (4) neutral nonpolar (hydrophobic) amino acids such as alanine, leucine, isoleucine, valine, proline, phenylalanine, tryptophan, and methionine. Conservative amino acid changes within defensin peptide sequences can be made by substituting one amino acid within one of these groups with another amino acid within the same group. Biologically functional equivalents of MD can have 10 or fewer conservative amino acid changes, seven or fewer conservative amino acid changes, or five, four, three, two, or one conservative amino acid changes. The encoding nucleotide sequence (e.g., gene, plasmid DNA, cDNA, or synthetic DNA) will thus have corresponding base substitutions, permitting it to encode biologically functional equivalent forms of the defensin peptides in the MD.

Functional fragments of any of the aforementioned or other defensin peptides used in MD can comprise defensin peptides having amino terminal deletions, carboxy terminal deletions, internal deletions, or any combination thereof. In certain embodiments, the functional fragment can contain at least one, two, three, four, five, six, or seven amino acid residue deletions from the amino terminus, the carboxy terminus, an internal region, or any combination thereof. In any of the aforementioned embodiments, the defensin peptide can comprise a conserved γ-core (gamma-core) amino acid sequence motif (NH2 . . . [GXC][X3-9C] . . . COOH). In certain embodiments, the peptide loop connecting the beta 2 and beta 3 strands of the gamma core motif can be mutagenized to increase the content of positively charged amino acid residues in the loop and increase the anti-fungal activity of the variant defensin (Sagaram et al., 2013).

Defensin chimeras comprising portions of any of the aforementioned or other defensins, defensin variants, or defensin fragments can also be used in the MD provided herein. In one embodiment, the chimeric defensin can comprise an MtDef4 peptide loop connecting the beta 2 and beta 3 strands of the gamma core motif that comprises the sequence RGFRRR (SEQ ID NO:106) or conservative substitutions thereof. Such conservative substitutions would include, but are not limited to, substitution of one or more arginyl residues in SEQ ID NO:106 with lysyl residues. A non-limiting example of a defensin chimera that could be used is an MsDef1/MsDef4 chimera wherein the MsDef1 peptide loop connecting the beta 2 and beta 3 strands of the gamma core motif are replaced with the MtDef4 peptide loop to obtain a defensin peptide with improved activity against Fusarium graminearum (Sagaram et al., 2011).

In any of the aforementioned or other embodiments, the variant defensin peptide can also comprise the cysteinyl residues that are involved in formation of disulfide bridges. In certain embodiments, cysteinyl residues involved in Cysl-Cys8, Cys2-Cys5, Cys3-Cys6, and Cys4-Cys7 disulfide bonding are retained in the variant defensin peptide. In certain embodiments, cysteinyl residues involved in Cysl-Cys8, Cys2-Cys5, Cys3-Cys6, and Cys4-Cys7 disulfide bonding are retained in the variant defensin peptide. In certain embodiments, one or more cysteinyl residues in the variant defensin peptide can be substituted with a distinct amino acid residue. In certain embodiments, one or more cysteinyl residues in the variant defensin peptide can be substituted with a serinyl or threonyl residue. In a MD containing variants of a mature MtDef4 protein (SEQ ID NO:76), substitutions of C3 S, C205, H33R, C41S, C43S, or any combination thereof can be used. Variants of single MtDef4 defensin peptides are disclosed in US Patent No. 7,825,297, which is incorporated herein by reference in its entirety. In a MD containing variants of a mature MtDef5 defensin peptide of SEQ ID NO:4 or SEQ ID NO:5, substitutions of one or more cysteinyl residues at positions 3, 14, 20, 24, 35, 44, 46, and/or 50 of SEQ ID NO:4 or SEQ ID NO:5 can be used. Variants of monomeric MtDef5 defensin proteins are disclosed in WO2014/179260, which is incorporated herein by reference in its entirety.

In certain embodiments, the permeability of a fungal plasma membrane treated with the multimeric defensin polypeptide is increased in comparison to permeability of a fungal plasma membrane treated with single defensin peptide of the multimeric defensin polypeptide. Membrane permeability can be measured by a variety of techniques that include, but are not limited to, dye uptake. Convenient dye uptake assays that can be used to assess changes in in membrane permeability include, but are not limited to, assays for uptake of Hoechst 33342 (H0342), rhodamine 123, SYTOX™ Green, and the like. These dyes enter into fungal cells only if their plasma membrane has been permeabilized by a defensin or other membrane-permeabilizing agent. Without seeking to be limited by theory, in certain embodiments it is believed that the multimeric defensin can provide improved fungal inhibition by increasing the permeability of treated fungal membranes in comparison to fungal membranes treated with a single, non-multimeric defensin peptide.

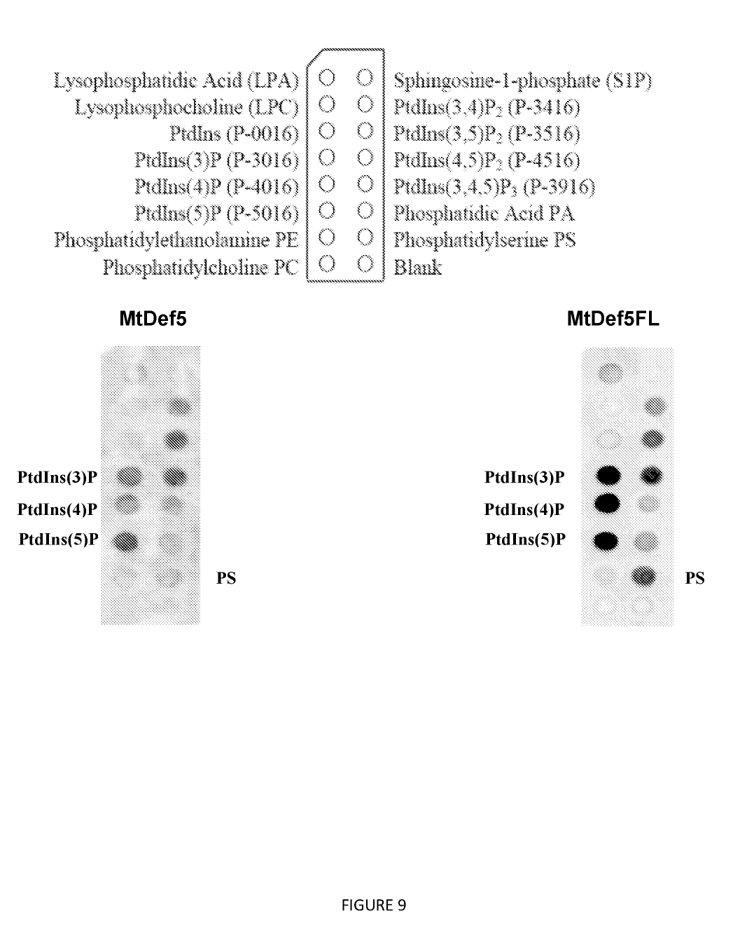

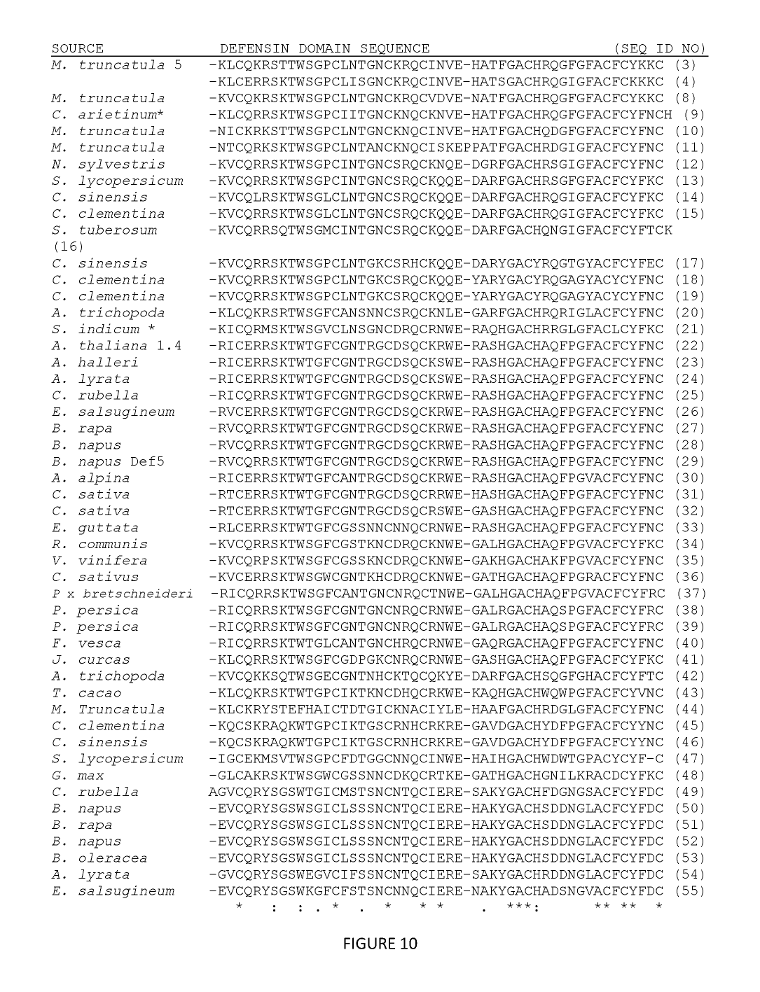

In certain embodiments, the defensin peptide or peptides used in the MD are defensin peptides that exhibit binding to a phospholipid and/or a sphingolipid. A summary of such defensin peptides, the plants in which they naturally occur, their IC50 values against Fusarium graminearum (concentration providing 50% inhibition of Fusarium graminearum growth), phospholipid binding specificity, and phospholipid binding strength is provided in Table 1. In certain embodiments, MD proteins provided herein comprised of any combination of MtDef4, MsDef1, NaD1, TPP3, MtDef5, RsAFP2, DmAMP1, Psd1 defensin peptides or variants thereof can exhibit lower IC50 values against one or more fungal pathogens, improved binding to phospholipids, improved binding to sphingolipids, or any combination thereof in comparison to a reference polypeptide containing just one of the defensin peptides that is contained in the MD. As shown in Table 1 and FIG. 9, the MtDef5FL1 polypeptide that comprises the MtDef5 defensin peptides A and B joined by a linker peptide exhibits both a lower IC50 value for Fusarium graminareum and improved binding to phosphatidyl monophosphates and phosphatidylserine in comparison to a polypeptide containing MtDef5 defensin peptide A. In certain embodiments, MD proteins comprised of any combination of MtDef4, MsDef1, NaD1, TPP3, MtDef5, RsAFP2, DmAMP1, Psd1 defensin peptides or variants thereof and various spacer peptides can be optimized for lower IC50 values against one or more fungal pathogens by selecting for MD having combinations of the defensin peptides and spacer peptides that provide for improved phospholipid and/or sphingolipid binding in comparison to a reference polypeptide containing just one of the defensin peptides that is contained in the MD. In certain embodiments, MD wherein the defensin peptides are the same or different and each peptide comprises an amino acid sequence at least 50%, 60%, 70%, 75%, 80%, 85%, 90%, 95%, 98%, 99%, or 100% identical to an amino acid sequence independently selected from the group consisting of SEQ ID NO:4, SEQ ID NO:5, SEQ ID NO:7-SEQ ID NO:55, functional fragments thereof, and chimeras thereof can be optimized for lower IC50 values against one or more fungal pathogens by selecting for MD having combinations of the defensin peptides and spacer peptides that provide for improved phospholipid binding in comparison to a reference polypeptide containing just one of the defensin peptides. Conservation of amino acid residues and motifs in the defensin peptides of SEQ ID NO:4, SEQ ID NO:5, and SEQ ID NO:7-SEQ ID NO:55 is provided in FIG. 10. In certain embodiments, such conservation can be used to design suitable defensin peptides for use in an MD that have at least 50%, 60%, 70%, 75%, 80%, 85%, 90%, 95%, 98%, 99%, or 100% identical to an amino acid sequence independently selected from the group consisting of SEQ ID NO:4, SEQ ID NO:5, SEQ ID NO:7-SEQ ID NO:55, functional fragments thereof, and chimeras thereof. Suitable assays for determining improved phospholipid and/or sphingolipid binding include, but are not limited to, protein-lipid overlay assays (e.g., Dowler et al., 2002), surface plasmon resonance assays (e.g., Baron and Pauron, 2014), biotin capture lipid affinity assays (e.g., Davidson et al., 2006), titration calorimetry assays (e.g., Miller and Cistola, 1993), and the like.

TABLE 1
Phospholipid binding defensin proteins

| | | IC₅₀ | Phospholipid-binding | Binding |
|---|---|---|---|---|
| Defensin | Plant | (nm)¹ | specificity | strength |
| MtDef4 | Medicago | 750- | Phosphatidic Acid | +++ |
| | truncatula | 1500 | | |
| MsDef1 | Medicago | 1500- | (1) phosphatidylinositol | (1)+ |
| | sativa | 3000 | monophosphates- | |
| | | | (PI3P, PI4P and PI5P) | |
| | | | (2) Phosphatidylinositol | (2)++ |
| | | | 3,5 bisphosphate | |
| | | | (3) phosphatidic acid | (3)+ |
| | | | (4) glucosylceramide | +++ |
| NaD1 | Nicotiana | 500 | (1) phosphatidylinositol | (1)+ |
| | alata | | monophosphates | |
| | | | (2) Phosphatidylinositol | (2)+++ |
| | | | 4,5 bisphosphate | |
| | | | (3) Phosphatidylinositol | (3)+ |
| | | | 3,5 bisphosphate | |
| | | | (4) Phosphatidylinositol | (4)+ |
| | | | 3,4 bisphosphate | |
| | | | (5) Phosphatidylinositol | (5)++ |
| | | | 3,4,5 triphosphate | |
| TPP3 | Tomato | 500 | Phosphatidylinositol 4,5 | +++ |
| | | | bisphosphate | |
| RsAFP2 | Raphanus | Not | Glucosylceramide | +++ |
| | sativum | available | | |
| DmAMP 1 | Dahlia | Not | Mannosyldiinositol | +++ |
| | merckii | available | phosphorylceramide | |
| Psd1 | Pisum | Not | Glucosylceramide | +++ |
| | sativum | available | | |
| MtDef5A | Medicago | 500-750 | (1) Phosphatidyl | (1)+ |
| (Peptide A; | truncatula | | monophosphates | |
| SEQ ID | | | (2) Phosphatidylinositol | (2)++ |
| NO: 5) | | | 4,5 bisphosphate | |
| | | | (3) Phosphatidylinositol | (3)++ |
| | | | 3,5 bisphosphate | |
| | | | (4) Phosphatidylinositol | (4)++ |
| | | | 3,4 bisphosphate | |
| | | | (5) Phosphatidylserine | (5)− |
| MtDef5FL1 | Medicago | 90 | (1) Phosphatidyl | (1)+++ |
| (SEQ ID | truncatula | | monophosphates | |
| NO: 7) | | | (2) Phosphatidylinositol | (2)++ |
| | | | 4,5 bisphosphate | |
| | | | (3) Phosphatidylinositol | (3)+ |
| | | | 3,5 bisphosphate | |
| | | | (4) Phosphatidylinositol | (4)+ |
| | | | 3,4 bisphosphate | |
| | | | (5) Phosphatidylserine | (5)++ |

¹IC₅₀ is for activity of the indicated protein against Fusarium graminareum.

Expression cassettes that provide for expression of the MD in monocotyledonous plants, dicotyledonous plants, or both can be constructed. Such MD expression cassette construction can be effected either in a plant expression vector or in the genome of a plant. Expression cassettes are DNA constructs wherein various promoter, coding, and polyadenylation sequences are operably linked. In general, expression cassettes typically comprise a promoter that is operably linked to a sequence of interest, which is operably linked to a polyadenylation or terminator region. In certain instances including, but not limited to, the expression of transgenes in monocot plants, it can also be useful to include an intron sequence. When an intron sequence is included it is typically placed in the 5′ untranslated leader region of the transgene. In certain instances, it can also be useful to incorporate specific 5′ untranslated sequences in a transgene to enhance transcript stability or to promote efficient translation of the transcript.

A variety of promoters can be used to express the MD proteins. One broad class of useful promoters are referred to as “constitutive” promoters in that they are active in most plant organs throughout plant development. For example, the promoter can be a viral promoter such as a CaMV35S or FMV35S promoter. The CaMV35S and FMV35S promoters are active in a variety of transformed plant tissues and most plant organs (e.g., callus, leaf, seed and root). Enhanced or duplicate versions of the CaMV35S and FMV35S promoters are particularly useful (U.S. Pat. No. 5,378,619, incorporated herein by reference in its entirety). Other useful promoters include the nopaline synthase (NOS) and octopine synthase (OCS) promoters (which are carried on tumor—inducing plasmids of A. tumefaciens), the cauliflower mosaic virus (CaMV) 19S promoters, a maize ubiquitin promoter, the rice Actl promoter, and the Figwort Mosaic Virus (FMV) 35 S promoter (see, e.g., U.S. Pat. No. 5,463,175, incorporated herein by reference in its entirety). It is understood that this group of exemplary promoters is non-limiting and that one skilled in the art could employ other promoters that are not explicitly cited here to express MD proteins.

Promoters that are active in certain plant tissues (i.e., tissue specific promoters) can also be used to drive expression of MD proteins and peptides. Expression of MD proteins and peptides in the tissue that is typically infected by a fungal pathogen is anticipated to be particularly useful. Thus, expression in reproductive tissues, seeds, roots, stems, or leaves can be particularly useful in combating infection of those tissues by certain fungal pathogens in certain crops. Examples of useful tissue-specific, developmentally regulated promoters include but are not limited to the β-conglycinin 7S promoter (Doyle et al., 1986), seed-specific promoters (Lam and Chua, 1991), and promoters associated with napin, phaseolin, zein, soybean trypsin inhibitor, ACP, stearoyl-ACP desaturase, or oleosin genes. Examples of root specific promoters include but are not limited to the RB7 and RD2 promoters described in U.S. Pat. Nos. 5,459,252 and 5,837,876, respectively.

Another class of useful promoters are promoters that are induced by various environmental stimuli. Promoters that are induced by environmental stimuli include, but are not limited to, promoters induced by heat (e.g., heat shock promoters such as Hsp70), promoters induced by light (e.g., the light-inducible promoter from the small subunit of ribulose 1,5-bisphosphate carboxylase, ssRUBISCO, a very abundant plant polypeptide), promoters induced by cold (e.g., COR promoters), promoters induced by oxidative stress (e.g., catalase promoters), promoters induced by drought (e.g., the wheat Em and rice rab16A promoters), and promoters induced by multiple environmental signals (e.g., rd29A promoters, Glutathione-S-transferase (GST) promoters).

Promoters that are induced by fungal infections in plants can also be used to drive expression of MD proteins and peptides. Useful promoters induced by fungal infections include those promoters associated with genes involved in phenylpropanoid metabolism (e.g., phenylalanine ammonia lyase, chalcone synthase promoters), genes that modify plant cell walls (e.g., hydroxyproline-rich glycoprotein, glycine-rich protein, and peroxidase promoters), genes encoding enzymes that degrade fungal cell walls (e.g., chitinase or glucanase promoters), genes encoding thaumatin-like protein promoters, or genes encoding proteins of unknown function that display significant induction upon fungal infection. Maize and flax promoters, designated as Mis1 and Fis1, respectively, are also induced by fungal infections in plants and can be used (US Patent Appl. Pub. No. 20020115849).

Depending on the fungus to which protection is sought, the present MD proteins and peptides can be expressed in any tissue or organ in the plant where the fungus attacks. In the case of Fusarium for example, a useful site for expression is in the roots. In the case of those fungi that infect by entering external plant surfaces, accumulation of the MD proteins and peptides in the apoplast can be used. In certain embodiments, the apoplast-localized MD can be expressed in roots, stems, leaves, etc., by the use of tissue-specific promoters.

Promoters active at particular developmental stages in the plant life cycle can also be used to optimize resistance to fungal infection and/or damage when it is most needed.

An intron can also be included in the DNA expression construct, especially in instances when the sequence of interest is to be expressed in monocot plants. For monocot plant use, introns such as the maize hsp70 intron (U.S. Pat. No. 5,424,412; incorporated by reference herein in its entirety), the maize ubiquitin intron, the Adh intron 1 (Callis et al., 1987), the sucrose synthase intron (Vasil et al., 1989) or the rice Actl intron (McElroy et al., 1990) can be used. Dicot plant introns that are useful include introns such as the CAT-1 intron (Cazzonnelli and Velten, 2003), the pKANNIBAL intron (Wesley et al., 2001; Collier et al., 2005), the PIV2 intron (Mankin et al., 1997) and the “Super Ubiquitin” intron (U.S. Pat. No. 6,596,925, incorporated herein by reference in its entirety; Collier et al., 2005) that have been operably integrated into transgenes. It is understood that this group of exemplary introns is non-limiting and that one skilled in the art could employ other introns that are not explicitly cited here to express MD proteins.

Certain embodiments comprise a sequence encoding an apoplast localization peptides that facilitates secretion of the mature MD proteins or peptides from plant cells. Apoplast localization peptides include, but are not limited to, peptides referred to as signal peptides. In certain embodiments, apoplast localization peptides can be operably linked to the n-termini of MD to provide for apoplast localization. Portions of the defensin proproteins that encode apoplast localization peptides (e.g., signal peptides) that can be used for secreting MD proteins or peptides from plant or other cells. Examples of defensin proproteins that contain apoplast localization peptides that can be used in MD include, but are not limited to, the defensin proproteins of SEQ ID NO:2, 56, 75, 78, 80, 82, 95, and 98, proteins that have at least about 70%, 80%, 90%, 95%, or 99% sequence identity to these sequences, and the biological functional equivalents of these sequences. Alternatively, signal peptide sequences derived from other Medicago defensin proteins (Hanks et al., 2005) can be used. Examples of such other Medicago defensin protein signal peptides include, but are not limited to, signal peptides of MtDef1.1 and MtDef2.1. Another example of a useful signal peptide encoding sequence that can be used in monocot plants is the signal peptide derived from a barley cysteine endoproteinase gene (Koehler and Ho, 1990) or an alpha-amylase gene. Another example of a useful signal peptide encoding sequence that can be used in dicot plants is the tobacco PRlb signal peptide. In other embodiments, wholly synthetic signal peptides can be used. This group of signal peptides is meant to be exemplary and non-limiting, and one skilled in the art could employ other signal peptides that are not explicitly cited here.

In other embodiments, sequences encoding peptides that provide for the localization of an MD in subcellular organelles can be operably linked to the sequences that encode the MD protein or peptide. MD proteins and peptides that are operably linked to a signal peptide are expected to enter the secretion pathway and can be retained by organelles such as the endoplasmic reticulum (ER) or targeted to the vacuole by operably linking the appropriate retention or targeting peptides to the C-terminus of the MD protein or peptide. Examples of vacuolar targeting peptides include, but are not limited to, a CTPP vacuolar targeting signal from the barley lectin gene. Examples of ER targeting peptides include, but are not limited to, a peptide comprising a KDEL amino acid sequence.

In certain embodiments, a plastid localization peptide can be operably linked to the MD to provide for localization of the MD in a plant plastid. Plastid transit peptides can be obtained from nuclear-encoded and plastid localized proteins that include, but are not limited to, Rubisco small subunit (RbcS), chlorophyll a/b-binding protein, ADP-glucose pyrophosphorylase (ADPGPP), and the like. Plastid targeting peptides that been disclosed in non-patent (Li and Teng, 2013) and patent literature (US Patent Appl. Pub. No. 20160017351 and U.S. Pat. No. 5,510,471, each incorporated herein by reference in their entireties). Chimeric plastid targeting peptides have also been disclosed (Lee et al., Plant Physiol., 2015). Any of the aforementioned plastic targeting peptides can be adapted for use in localizing MD proteins in plastids. In certain embodiments, the plastid localization peptide can be operably linked to the N-terminus of the MD.

In certain embodiments, a mitochondrial localization peptide can be operably linked to the MD to provide for localization of the MD in the mitochondria. Mitochondrial localization peptides can be obtained from nuclear-encoded and mitochondrial localized proteins that include, but are not limited to, beta-subunit of the F(1)-ATP synthase, alternative oxidases, and the gamma-subunit of the F(1)-ATP synthase. Mitochondrial targeting peptides have been disclosed (Sjoling and Glaser; 1998; Huang et al., Plant Physiology, 2009). In certain embodiments, the mitochondrial localization peptide will be operably linked to the N-terminus of the MD. Any of the aforementioned mitochondrial targeting peptides can be adapted for use in localizing MD proteins in mitochondria. In certain embodiments, the mitochondrial localization peptide can be operably linked to the N-terminus of the MD.

In still other embodiments, dual localization peptide(s) can be used to provide for localization of the MD in both plastids and mitochondria (Carrie and Small, 2013).

Localization of MD proteins and peptides in the apoplast endoplasmic reticulum, the vacuole, plastids, or mitochondria can provide for useful properties such as increased expression in transgenic plants and/or increased efficacy in inhibiting fungal growth in transgenic plants. In certain embodiments, the localization peptide is a heterologous localization peptide that can direct an operably associated polypeptide to an extracellular or sub-cellular location that is different than the extracellular or sub-cellular location of a naturally occurring polypeptide comprising one or both of the defensin peptides. In certain embodiments, the localization peptide can target an MD that comprises a spacer peptide, linker peptide, or junction sequence that is susceptible to cleavage by a plant endoproteinase to an extracellular or sub-cellular compartment where activity of that plant endoproteinase reduced or absent and thus provide for accumulation of the MD in the transgenic plant.

In other embodiments, the MD-, defensin-, localization-, spacer-, or other polypeptide encoding nucleotide sequence can be synthesized de novo from an MD protein or peptide sequence disclosed herein. The sequence of the polypeptide-encoding nucleotide sequence can be deduced from the MDD-, defensin-, localization-, spacer-, or other polypeptide sequence through use of the genetic code. Computer programs such as “BackTranslate” (GCG™ Package, Acclerys, Inc. San Diego, Calif.) can be used to convert a protein or peptide sequence to the corresponding nucleotide sequence that encodes the protein or peptide.

Furthermore, the synthetic MD-, defensin-, localization-, spacer-, or other polypeptide nucleotide sequence can be designed so that it will be optimally expressed in plants. U.S. Pat. No. 5,500,365 describes a method for synthesizing plant genes to optimize the expression level of the protein encoded by the synthesized gene. This method relates to the modification of the structural gene sequences of the exogenous transgene, to make them more “plant-like” and therefore more efficiently transcribed, processed, translated, and expressed by the plant. Features of genes that are expressed well in plants include use of codons that are commonly used by the plant host and elimination of sequences that can cause undesired intron splicing or polyadenylation in the coding region of a gene transcript. A similar method for obtaining enhanced expression of transgenes in monocotyledonous plants is disclosed in U.S. Pat. No. 5,689,052.

In certain embodiments, an MD encoding sequence can also be operably linked to a 3′ non-translated region containing a polyadenylation signal. This polyadenylation signal provides for the addition of a polyadenylate sequence to the 3′ end of the RNA. The Agrobacterium tumor-inducing (Ti) plasmid nopaline synthase (NOS) gene 3′ and the pea ssRUBISCO E9 gene 3′ un-translated regions contain polyadenylate signals and represent non-limiting examples of such 3′ untranslated regions that can be used. It is understood that this group of polyadenylation regions is non-limiting and that one skilled in the art could employ other polyadenylation regions that are not explicitly cited here.

The DNA constructs that comprise the plant expression cassettes described above can either be constructed in the plant genome by using site specific insertion of heterologous DNA into the plant genome, by mutagenizing the plant genome, and/or by introducing the expression cassette into the plant genome with a vector or other DNA transfer method. Vectors contain sequences that provide for the replication of the vector and covalently linked sequences in a host cell. For example, bacterial vectors will contain origins of replication that permit replication of the vector in one or more bacterial hosts. Agrobacterium-mediated plant transformation vectors typically comprise sequences that permit replication in both E.coli and Agrobacterium as well as one or more “border” sequences positioned so as to permit integration of the expression cassette into the plant chromosome. Such Agrobacterium vectors can be adapted for use in either Agrobacterium tumefaciens or Agrobacterium rhizogenes. Selectable markers encoding genes that confer resistance to antibiotics are also typically included in the vectors to provide for their maintenance in bacterial hosts.

Methods of obtaining a transgenic plant capable of inhibiting growth of a plant pathogenic fungus are also provided. In one embodiment, expression vectors suitable for expression of the MD protein or peptide in various dicot and monocot plants are introduced into a plant, a plant cell, a protoplast, or a plant tissue using transformation techniques as described herein. In another embodiment, the MD expression cassette is constructed in the plant genome by using site specific insertion of heterologous DNA into the plant genome by mutagenizing the plant genome. Next, a transgenic plant containing or comprising the MD expression vector is obtained by regenerating that transgenic plant from the plant, plant cell, protoplast, or plant tissue that received the expression vector. The final step is to obtain a transgenic plant that expresses a plant pathogenic fungus inhibitory amount of the mature MD protein or peptide, where a “plant pathogenic fungus inhibitory amount” is a level of MD protein or peptide sufficient to provide any measurable decrease in fungal growth in the transgenic plant and/or any measurable decrease in the adverse effects caused by fungal growth in the transgenic plant.

Any of the MD expression vectors can be introduced into the chromosomes of a host plant via methods such as Agrobacterium-mediated transformation, Rhizobium-mediated transformation, Sinorhizobium-mediated transformation, particle-mediated transformation, DNA transfection, DNA electroporation, or “whiskers”-mediated transformation. The aforementioned methods of introducing transgenes are described in US Patent Appl. Pub. No. 20050289673 (Agrobacterium-mediated transformation of corn), U.S. Pat. No. 7,002,058 (Agrobacterium-mediated transformation of soybean), U.S. Pat. No. 6,365,807 (particle mediated transformation of rice), and U.S. Pat. No. 5,004,863 (Agrobacterium-mediated transformation of cotton), each of which are incorporated herein by reference in their entirety. Methods of using bacteria such as Rhizobium or Sinorhizobium to transform plants are described in Broothaerts, et al., 2005. It is further understood that the MD expression vector can comprise cis-acting site-specific recombination sites recognized by site-specific recombinases, including Cre, Flp, Gin, Pin, Sre, pinD, Int-B13, and R. Methods of integrating DNA molecules at specific locations in the genomes of transgenic plants through use of site-specific recombinases can then be used (U.S. Pat. No. 7,102,055). Those skilled in the art will further appreciate that any of these gene transfer techniques can be used to introduce the expression vector into the chromosome of a plant cell, a protoplast, a plant tissue, or a plant.

Methods of introducing plant mini-chromosomes comprising plant centromeres that provide for the maintenance of the recombinant mini-chromosome in a transgenic plant (U.S. Pat. Nos. 6,972,197 and 8,435,783) can also be used to introduce and maintain MD in such plants. In these embodiments, the transgenic plants harbor the mini-chromosomes as extrachromosomal elements that are not integrated into the chromosomes of the host plant.

In certain embodiments, transgenic plants can be obtained by linking the gene of interest (in this case an MD-encoding nucleotide sequence) to a selectable marker gene, introducing the linked transgenes into a plant cell, a protoplast, a plant tissue, or a plant by any one of the methods described above, and regenerating or otherwise recovering the transgenic plant under conditions requiring expression of the selectable marker gene for plant growth. The selectable marker gene can be a gene encoding a neomycin phosphotransferase protein, a phosphinothricin acetyltransferase protein, a glyphosate resistant 5-enol-pyruvylshikimate-3-phosphate synthase (EPSPS) protein, a hygromycin phosphotransferase protein, a dihydropteroate synthase protein, a sulfonylurea insensitive acetolactate synthase protein, an atrazine insensitive Q protein, a nitrilase protein capable of degrading bromoxynil, a dehalogenase protein capable of degrading dalapon, a 2,4-dichlorophenoxyacetate monoxygenase protein, a methotrexate insensitive dihydrofolate reductase protein, or an aminoethylcysteine insensitive octopine synthase protein. The corresponding selective agents used in conjunction with each gene can be: neomycin (for neomycin phosphotransferase protein selection), phosphinotricin (for phosphinothricin acetyltransferase protein selection), glyphosate (for glyphosate resistant 5-enol-pyruvylshikimate-3-phosphate synthase (EPSPS) protein selection), hygromycin (for hygromycin phosphotransferase protein selection), sulfadiazine (for a dihydropteroate synthase protein selection), chlorsulfuron (for a sulfonylurea insensitive acetolactate synthase protein selection), atrazine (for an atrazine insensitive Q protein selection), bromoxinyl (for a nitrilase protein selection), dalapon (for a dehalogenase protein selection), 2,4-dichlorophenoxyacetic acid (for a 2,4-dichlorophenoxyacetate monoxygenase protein selection), methotrexate (for a methotrexate insensitive dihydrofolate reductase protein selection), or aminoethylcysteine (for an aminoethylcysteine insensitive octopine synthase protein selection).

In certain embodiments, a transgene plant comprising an MD can be obtained by using techniques that provide for site specific insertion of heterologous DNA into the genome of a plant. In certain embodiments, the a DNA fragment comprising at least one of a defensin peptide, a spacer peptide that is resistant to cleavage by a plant endoproteinase, a heterologous promoter, or a heterologous localization peptide, is site specifically integrated into the genome to a plant cell, tissue, part, or whole plant to create a sequence within that genome that encodes a MD. In one embodiment of the method, the heterologous DNA encodes a spacer peptide sequence that is used to replace the endogenous DNA sequence encoding a linker peptide that joins two encoded defensin to provide a transgenic plant comprising genomic DNA encoding endogenous defensin peptides that are operably linked to a heterologous spacer peptide encoding DNA sequence. In one embodiment of the method, the heterologous DNA encodes a spacer peptide sequence and a defensin peptide that is inserted in-frame at either the N-terminus of the endogenous defensin peptide coding region or at the C-terminus of the defensin peptide coding region to provide a transgenic plant comprising genomic DNA encoding an endogenous defensin peptide that is operably linked to a heterologous spacer peptide encoding DNA sequence and a defensin peptide. In certain embodiments where a heterologous DNA that encodes a spacer peptide sequence and a defensin peptide is inserted in frame with an endogenous defensin encoding sequence, the inserted and defensin peptide can identical to the endogenous defensin peptide or a variant of the endogenous defensin peptide. In one embodiment of the method, the heterologous DNA encodes a heterologous localization peptide that is inserted in frame with a genomic coding region that comprises endogenous defensin peptides that are joined by an endogenous linker peptide to provide a transgenic plant comprising genomic DNA encoding the endogenous defensin peptides and the endogenous linker peptide with the localization peptide operably linked to the encoded peptide. In certain embodiments, the localization peptide will provide for localization of the peptide encoding the two endogenous defensin peptides and the linker peptide in an extracellular or sub-cellular location where activity of a plant endoproteinase that can cleave the peptide linker is reduced or absent. In certain embodiments, a heterologous promoter or promoter element can be inserted at or near the 5′ end of a genomic region that comprises a sequence encoding an endogenous defensin peptide, an endogenous defensin peptide that is joined to another endogenous defensin peptide with a linker peptide, an endogenous defensin peptide that is joined to another defensin peptide with a heterologous spacer peptide, a defensin peptide that is operably linked to a heterologous localization peptide, or any combination thereof to obtain a transgenic plant where the genomic region is under the transcriptional control of the inserted or composite promoter. In practicing any of the aforementioned methods, such heterologous DNA can either be inserted in a parallel (e.g., at the same time) or sequentially (e.g., at the distinct times). In one non-limiting example, a heterologous DNA encoding a spacer peptide and a defensin peptide can be inserted into an endogenous genomic region encoding an endogenous defensin peptide at the same time that a heterologous promoter, promoter element, and/or localization peptide is inserted into the genomic region. In another non-limiting example, a heterologous DNA encoding a spacer peptide and a defensin peptide can be inserted into an endogenous genomic region encoding an endogenous defensin peptide to obtain a first transgenic plant comprising genomic DNA encoding an endogenous defensin peptide that is operably linked to the heterologous spacer peptide encoding DNA sequence and the defensin peptide. A heterologous promoter, promoter element, and/or localization peptide can then be inserted into the genomic DNA of the first genomic plant to a first transgenic plant comprising genomic DNA encoding an endogenous defensin peptide that is operably linked to the heterologous spacer peptide encoding DNA sequence and the defensin peptide as well as to the heterologous promoter, promoter element, and/or localization peptide. Examples of methods for inserting foreign DNA at specific sites in the plant genome with site-specific nucleases such as meganucleases or zinc-finger nucleases are at least disclosed in Voytas, 2013. Examples of methods for inserting foreign DNA into the plant genome with clustered regularly interspaced short palindromic repeats (CRISPR)-associated (Cas)-guide RNA technology and a Cas endonuclease are at least disclosed by Svitashev et al., 2015; Kumar and Jain, 2015; and in US Patent Appl. Pub. No. 20150082478, which is specifically incorporated herein by reference in its entirety.

In certain embodiments, a genetically edited plant comprising an MD can be obtained by using techniques that provide for genome editing in the plant. In one embodiment, a plant comprising an endogenous gene encoding two defensin peptides that are joined by a linker peptide encoding region can be subjected to a genome editing technique where the linker peptide is mutagenized to convert the linker peptide to a spacer peptide that is resistant to cleavage by an endogenous plant endoproteinase. Examples of endogenous plant defensins that comprise two defensin peptides that are separated by linker peptides that could be converted to spacer peptides include, but are not limited to, SEQ ID NO:56, SEQ ID NO:95, and SEQ ID NO:98. Examples of methods for plant genome editing with clustered regularly interspaced short palindromic repeats (CRISPR)-associated (Cas)-polynucleotide modification template technology and a Cas endonuclease are at least disclosed by Svitashev et al., 2015; Kumar and Jain, 2015; and in US Patent Appl. Pub. No. 20150082478, which is specifically incorporated herein by reference in its entirety.

Transgenic plants can also be obtained by linking a gene of interest (in this case an MD-encoding nucleotide sequence) to a scoreable marker gene, introducing the linked transgenes into a plant cell by any of the methods described above, and regenerating the transgenic plants from transformed plant cells that test positive for expression of the scoreable marker gene. The scoreable marker gene can be a gene encoding a beta-glucuronidase protein, a green fluorescent protein, a yellow fluorescent protein, a beta-galactosidase protein, a luciferase protein derived from a luc gene, a luciferase protein derived from a lux gene, a sialidase protein, streptomycin phosphotransferase protein, a nopaline synthase protein, an octopine synthase protein, or a chloramphenicol acetyl transferase protein.

When an expression vector encoding an MD is introduced into a plant cell or plant tissue or when an MD is introduced in the genome of a plant cell or tissue by site specific insertion of heterologous DNA into the plant genome, the transformed cells or tissues can be regenerated into whole plants by culturing these cells or tissues under conditions that promote the formation of a whole plant (i.e., the process of regenerating leaves, stems, roots, and, in certain plants, reproductive tissues). The development or regeneration of transgenic plants from either single plant protoplasts or various explants has been described (Horsch, R. B. et al., 1985). This regeneration and growth process typically includes the steps of selection of transformed cells and culturing selected cells under conditions that will yield rooted plantlets. The resulting transgenic rooted shoots are thereafter planted in an appropriate plant growth medium such as soil. Alternatively, transgenes can also be introduced into isolated plant shoot meristems and plants regenerated without going through callus stage tissue culture (U.S. Pat. No. 7,002,058). When the transgene is introduced directly into a plant, or more specifically into the meristematic tissue of a plant, seed can be harvested from the plant and selected or scored for presence of the transgene. In the case of transgenic plant species that reproduce sexually, seeds can be collected from plants that have been “selfed” (self-pollinated) or out-crossed (i.e., used as a pollen donor or recipient) to establish and maintain the transgenic plant line. Transgenic plants that do not sexually reproduce can be vegetatively propagated to establish and maintain the transgenic plant line. In certain embodiments, transgenic plants are derived from a transformation event where the transgene has inserted into one or more locations in the plant genome. In certain embodiments, a seed produced by the transgenic plant, a progeny from such seed, and a seed produced by the progeny of the original transgenic plant are provided. Such progeny and seeds will have an MD protein-encoding transgene stably incorporated into their genome, and such progeny plants will inherit the traits afforded by the introduction of a stable transgene in Mendelian fashion. It is further recognized that transgenic plants containing the MD encoding DNA constructs described herein, and materials derived therefrom, can be identified through use of PCR or other methods that can specifically detect the sequences in the DNA constructs.

Once a transgenic plant is regenerated or recovered, a variety of methods can be used to identify or obtain a transgenic plant that expresses a plant pathogenic fungus inhibitory amount of MD. One general set of methods is to perform assays that measure the amount of MD that is produced. For example, various antibody-based detection methods employing antibodies that recognize MD can be used to quantitate the amount of MD produced. Examples of such antibody-based assays include, but are not limited to, ELISAs, RIAs, or other methods wherein an MD-recognizing antibody is detectably labelled with an enzyme, an isotope, a fluorophore, a lanthanide, and the like. By using purified or isolated MD protein or peptide as a reference standard in such assays (i.e., providing known amounts of MD), the amount of MD present in the plant tissue in a mole per gram of plant material or mass per gram of plant material can be determined. The MD protein or peptide will typically be expressed in the transgenic plant at the level of “parts per million” or “PPM”, where microgram levels of MD are present in gram amounts of fresh weight plant tissue. In this case, 1 microgram of MD per 1 gram of fresh weight plant tissue would represent a MD concentration of 1 PPM. A plant pathogenic fungus inhibitory amount of MD protein or peptide is at least about 0.05 PPM (i.e., 0.05 μg MD protein or peptide per gram fresh weight plant tissue) or at least about 0.1 PPM. In certain embodiments, a plant pathogenic fungus inhibitory amount of MD is at least about 0.5 PPM. In certain embodiments, the amount of MD is at least about 1.0 PPM. In certain embodiments, the amount of MD protein or peptide is at least about 2.0 PPM. In certain embodiments, the amount of the MD protein is at least about 0.05 PPM, 0.1 PPM, 0.5 PPM, or 1.0 PPM to about 5, 10, 20, 50, or 100 PPM.

Alternatively, the amount of MD-encoding mRNA produced by the transgenic plant can be determined to identify plants that express plant pathogenic fungus inhibitory amounts of MD. Techniques for relating the amount of protein produced to the amount of RNA produced include methods such as constructing a standard curve that relates specific RNA levels (i.e., MD mRNA) to levels of the MD protein or peptide (determined by immunologic or other methods). Methods of quantitating MD mRNA typically involve specific hybridization of a polynucleotide to either the MD mRNA or to a cDNA (complementary DNA) or PCR product derived from the MD RNA. Such polynucleotide probes can be derived from either the sense and/or antisense strand nucleotide sequences of the MD-encoding transgene. Hybridization of a polynucleotide probe to the MD mRNA or cDNA can be detected by methods including, but not limited to, use of probes labelled with an isotope, a fluorophore, a lanthanide, or a hapten such as biotin or digoxigenin. Hybridization of the labelled probe can be detected when the MD RNA is in solution or immobilized on a solid support such as a membrane. When quantitating MD RNA by use of a quantitative reverse-transcriptase Polymerase Chain Reaction (qRT-PCR), the PCR product can be detected by use of any of the aforementioned labelled polynucleotide probes, by use of an intercalating dye such as ethidium bromide or SYBR green, or use of a hybridization probe containing a fluorophore and a quencher such that emission from the fluorophore is only detected when the fluorophore is released by the 5 nuclease activity of the polymerase used in the PCR reaction (i.e., a TaqMan™ reaction; Applied Biosystems, Foster City, Calif.) or when the fluorophore and quencher are displaced by polymerase mediated synthesis of the complementary strand (i.e., Scorpion™ or Molecular Beacon™ probes) .Various methods for conducting qRT-PCR analysis to quantitate mRNA levels are well characterized (Bustin, S. A.; 2002). Fluorescent probes that are activated by the action of enzymes that recognize mismatched nucleic acid complexes (i.e., Invader™, Third Wave Technologies, Madison, Wis.) can also be used to quantitate RNA. Those skilled in the art will also understand that RNA quantitation techniques such as Quantitative Nucleic Acid Sequence Based Amplification (Q-NASBA™) can be used to quantitate MD-encoding mRNA and identify expressing plants.

Transgenic plants that express plant pathogenic fungus inhibitory amounts of MD proteins or peptides can also be identified by directly assaying such plants for inhibition of the growth of a plant pathogenic fungus. Such assays can be used either independently or in conjunction with MDD expression assays to identify the resistant transgenic plants.

Infection of certain plants with certain plant pathogen fungi can result in distinctive effects on plant growth that are readily observed. Consequently, one can distinguish MD-expressing transgenic plants by simply challenging such plants transformed with MD-encoding transgenes with pathogenic plant fungi and observing reduction of the symptoms normally associated with such infections. Such observations are facilitated by co-infecting otherwise identical, non-transgenic control plants that do not contain an MD encoding transgene with the same type and dose of plant pathogenic fungi used to infect the transgenic plants that contain an MD-encoding transgene. Identification of transgenic plants that control or combat fungal infection can be based on observation of decreased disease symptoms, measurement of the decreased fungal growth in the infected plant (e.g., by determining the numbers of colony forming units per gram of infected tissue) and/or by measurement of the amount of mycotoxins present in infected plant tissue. The use of fungal disease severity assays and colony formation assays in conjunction with expression assays to identify transgenic MsDef1 -expressing potato plants that are resistant to Verticillium dahliae has been described (U.S. Pat. No. 6,916,970 and Gao et al., 2000). It is similarly anticipated that a variety of MD-expressing transgenic plants that combat or control fungal pathogens can be identified by scoring transgenic plants for resistance to fungal pathogens that infect those plants. Examples of MD transgene-conferred fungal resistance that can be assayed by observing reductions in disease symptoms or reductions in fungal growth include, but are not limited to, resistance of transgenic corn to Fusarium verticillioides, Fusarium moniliforme, Colletotricum graminicola, Stenocarpella maydis, and/or Cercospora zeae-maydis; resistance of transgenic wheat to head blight (Fusarium graminearum), powdery mildew (Erysiphe graminis f sp. tritici), stripe rust, stem rust or leaf rust (Puccinia recondita f sp. tritici); resistance of transgenic cotton to Fusarium oxysporum and Verticillium dahlia; resistance of transgenic rice to Magnaporthe grisea and Rhizoctonia solani, and resistance of transgenic soybean to Asian Soybean rust (Phakopsora pachyrhizi), Phytophthora Root Rot (Phytophthora sp.), White Mold (Sclerotinia sp.), Sudden Death Syndrome (Fusarium solani) and/or Brown Stem Rot (Phialophora gregata).

Transgenic plants that express plant pathogenic fungus inhibitory amounts of MD can also be identified by measuring decreases in the adverse effects cause by fungal growth in such plants. Such decreases can be ascertained by comparing the extent of the adverse effect in an MD-expressing transgenic plant relative to an otherwise identical, non-transgenic control plant that does not express MD. Adverse effects of fungal growth in a plant that can be measured include any type of plant tissue damage or necrosis, any type of plant yield reduction, any reduction in the value of the crop plant product, and/or production of undesirable fungal metabolites or fungal growth by-products including, but not limited to, mycotoxins. Mycotoxins comprise a number of toxic molecules produced by fungal species, including but not limited to polyketides (including aflatoxins, demethylsterigmatocystin, O-methylsterigmatocystin, etc.), fumonisins, alperisins (e.g., Als A2, Bls B2), sphingofungins (A, B, C and D), trichothecenes, fumifungins, and the like. Methods of quantitating mycotoxin levels are widely documented. Moreover, commercial kits for measurement of the mycotoxins such as aflatoxin, fumonisin, deoxynivalenol, and zearalenone are also available (VICAM, Watertown, Mass., USA).

A wide variety of plants that express MD can either be constructed by using site specific insertion of heterologous DNA into the plant genome, by mutagenizing the plant genome, and/or by introducing the expression cassette into the plant genome with a vector or other DNA transfer method to obtain transgenic plants that combat or control fungal infections, or that resist such infections.

Plants of interest include both food crop plants and biofuels or energy crop plants, as listed above. Transgenic monocot plants obtainable by the expression vectors and methods described herein include but are not limited to barley, corn, flax, oat, rice, rye, sorghum, turf grass, sugarcane, and wheat. Transgenic dicot plants obtainable by the expression vectors and methods described herein include but are not limited to alfalfa, Arabidopsis, barrel medic, banana, broccoli, bean, cabbage, canola, carrot, cassava, cauliflower, celery, citrus, cotton, cucurbits, eucalyptus, garlic, grape, onion, lettuce, pea, peanut, pepper, potato, poplar, pine, sunflower, safflower, soybean, strawberry, sugar beet, sweet potato, tobacco, and tomato.

Expression of MD proteins and peptides in yeast is also specifically contemplated herein. The construction of expression vectors for production of heterologous proteins in various yeast genera is well established. In general, such expression vectors typically comprise a promoter that is operably linked to a sequence of interest which is operably linked to a polyadenylation or terminator region. Examples of yeast genera that have been used to successfully express heterologous genes include Candida, Kluveromyces, Hansuela, Pichia, Saccharomyces, Schizosaccharomyces, and Yarrowia. A general description of expression vectors and transformation systems for Saccharomyces is found in Kingsman et al (1985). Expression vectors and transformation systems useful for yeasts other than Saccharomyces are described in Reiser et al (1990).

In general, the promoter and polyadenylation region are selected based on their operability in a given yeast host. For example, the AOX1 or AOX2 promoters of Pichia can be used in conjunction with the AOX1, AOX2, p40, or p76 polyadenylation sequences of Pichia to express a heterologous protein such as an MD protein or peptide. Both the AOX1 and AOX2 promoters are particularly useful in Pichia as both promoters provide for abundant expression of the linked heterologous gene when induced by addition of methanol to the growth medium. The use of these Pichia promoters and polyadenylation sequences is described in U.S. Pat. No. 4,855,231, which is expressly incorporated herein by reference in its entirety. Similarly, the Hansuela MOX, DHAS, or FMDH promoters can be used to express heterologous proteins such as MD in Hansuela. The MOX, DHAS, or FMDH promoters are particularly useful in Hansuela as these promoters provide for abundant expression of the linked heterologous gene when induced by addition of methanol to the growth medium. The use of the MOX and DHAS promoters in Hansuela is described in U.S. Pat. No. 5,741,672, while the use of the FMDH promoter in Hansuela is described in U.S. Pat. No. 5,389,525, each of which is expressly incorporated herein by reference in its entirety. For Kluveromyces, a Lactase promoter and polyadenylation sequence can be used to express heterologous genes such as MD. Expression of heterologous genes that are operably linked to the Lactase promoter and polyadenylation sequence is achieved by growing Kluveromyces in the presence of galactose. The use of the Lactase promoter and polyadenylation sequences in Kluveromyces is described in U.S. Pat. No. 6,602,682, which is expressly incorporated herein by reference in its entirety.

Yeast expression vectors that provide for secretion of heterologous proteins such as MD into the growth medium by transformed yeast are also contemplated. Secretion of the mature MD protein or peptide is typically achieved by operable linkage of a signal peptide sequence or a signal peptide and propeptide sequence to the mature MD protein- or peptide-encoding sequence. Examples of useful signal peptides for secretion of heterologous proteins in yeast include but are not limited to an alpha-factor signal peptide, an invertase signal peptide, and a PHO1 signal peptide, all of which are derived from yeast. The alpha-factor signal peptide is typically derived from Saccharomyces, Kluveromyces, or Candida, while the PHO1 signal peptide is derived from Pichia.

A particularly useful signal peptide sequence or signal peptide and propeptide sequence for secretion of proteins in yeast is derived from the S. cerevisiae alpha-factor, and is described in U.S. Pat. Nos. 4,546,082, 4,588,684, 4,870,008, and 5,602,034, each of which is expressly incorporated herein by reference in its entirety. The S. cerevisiae alpha-factor signal peptide and propeptide sequence consist of amino acids 1-83 of the primary, unprocessed translation product of the S. cerevisiae alpha mating factor gene (GenBank Accession Number: P01149). In certain embodiments, the signal peptide sequence of the alpha-mating factor comprising amino acids 1 to about 19 to 23 of the alpha-mating factor proprotein can be directly linked to the N-terminus of the mature MD protein to provide for secretion of mature MD protein. In this case, the signal peptide is cleaved from the mature MD protein in the course of the secretion process. Alternatively, the signal peptide and propeptide of the alpha mating factor can be operably linked to the mature MD encoding sequence via a cleavage site sequence. This cleavage site sequence can comprise a variety of sequences that provide for proteolytic processing of the leader sequence and gene of interest. In the native S. cerevisiae alpha mating factor gene the s cleavage site sequence corresponds to amino acid residues 84-89 and is represented by the sequence Lys84-Arg85-Glu86-Ala87-Glu88-Ala 89 (SEQ ID NO:107). The sequence Lys-Arg corresponds to a KEX2 protease recognition site while the Glu-Ala-Glu-Ala sequence corresponds to a duplicated dipeptidylammopeptidase or STE13 recognition site. In certain embodiments, a DNA fragment encoding the 89 amino acid S. cerevisiae alpha factor signal, propeptide coding region, and entire native spacer coding region (i.e., the N-terminal 89 amino acid residues of the alpha mating factor precursor protein containing both the Lys-Arg KEX2 protease cleavage site at residues 84 and 85 as well as the Glu-Ala-Glu-Ala dipeptidylammopeptidase or STE13 recognition site at residues 86-89) is operably linked to the sequence encoding the mature MD protein. When the N-terminal 89 amino acids of the alpha mating factor precursor protein are fused to the N-terminus of a heterologous protein such as MD, the propeptide sequence is typically dissociated from the heterologous protein via the cleavage by endogenous yeast proteases at either the KEX2 or STE13 recognition sites. In other embodiments, a DNA fragment encoding the smaller 85 amino acid Saccharomyces cerevisiae alpha factor signal peptide, propeptide, and KEX2 spacer element (i.e., the N-terminal 85 amino acid residues of the alpha mating factor precursor protein containing just the Lys-Arg KEX2 protease cleavage site at residues 84 and 85) is operably linked to the sequence encoding the mature MD protein. When the N-terminal 85 amino acids of the alpha mating factor precursor protein are fused to the N-terminus of a heterologous protein such as MD, the propeptide sequence is typically dissociated from the heterologous protein via cleavage by endogenous yeast proteases at the KEX2 recognition site. The MD protein can thus be expressed without the glu-ala repeats.

To obtain transformed yeast that express MD proteins and peptides, the yeast MD expression cassettes (e.g., yeast promoter, yeast signal peptide encoding sequence, mature MD protein sequence, and polyadenylation sequence) are typically combined with other sequences that provide for selection of transformed yeast. Examples of useful selectable marker genes include, but are not limited to, genes encoding a ADE protein, a HISS protein, a HIS4 protein, a LEU2 protein, a URA3 protein, ARG4 protein, a TRP1 protein, a LYS2 protein, a protein conferring resistance to a bleomycin or phleomycin antibiotic, a protein conferring resistance to chloramphenicol, a protein conferring resistance to G418 or geneticin, a protein conferring resistance to hygromycin, a protein conferring resistance to methotrexate, an a AR04-OFP protein, and a FZF1-4 protein.

DNA molecules comprising the yeast MD expression cassettes and selectable marker genes are introduced into yeast cells by techniques such as transfection into yeast spheroplasts or electroporation. In certain embodiments, the DNA molecules comprising the yeast MD expression cassettes and selectable marker genes are introduced as linear DNA fragments that are integrated into the genome of the transformed yeast host cell. Integration can occur either at random sites in the yeast host cell genome or at specific sites in the yeast host cell genome. Integration at specific sites in the yeast host cell genome is typically accomplished by homologous recombination between sequences contained in the expression vector and sequences in the yeast host cell genome. Homologous recombination is typically accomplished by linearizing the expression vector within the homologous sequence (for example, within the AOX1 promoter sequence of a Pichia expression vector when integrating the expression vector into the endogenous AOX1 gene in the Pichia host cell). In other embodiments, the yeast expression cassettes can also comprise additional sequences such as autonomous replication sequences (ARS) that provide for the replication of DNA containing the expression cassette as an extrachromosomal (non-integrated) element. Such extra-chromosomal elements are typically maintained in yeast cells by continuous selection for the presence of the linked selectable marker gene. Yeast artificial chromosomes (YACs) containing sequences that provide for replication and mitotic transmission are another type of vector that can be used to maintain the DNA construct in a yeast host.

Yeast cells transformed with the yeast MD expression cassettes can be used to produce MD proteins and peptides. These MD molecules can be used directly as antifungal agents, to produce antifungal compositions that can be applied to plants, as immunogens to raise antibodies that recognize the MD proteins or peptides, or as reference standards in kits for measuring concentrations of MD proteins and peptides in various samples. The transformed yeast cells expressing MD antifungal molecules can also be applied to plants to combat/control pathogenic fungal infections. The methods of producing MD proteins and peptides typically first comprise the step of culturing yeast cells transformed with MD expression cassettes under conditions wherein the yeast cells express a mature MD molecule. In general, the conditions where the yeast cells express the mature MD molecules are conditions that allow for or specifically induce expression of the yeast promoter that is operably linked to the MD coding sequence in the yeast expression cassette. When the yeast is Pichia and the signal-peptide/MD gene is under the control of an AOX1 or AOX2 promoter, addition of methanol to the growth medium will provide for expression of mature MD protein. Similarly, when the yeast is Hansuela and the signal-peptide/MD gene is under the control of a MOX, DHAS, or FMDH promoter, addition of methanol to the growth medium will provide for expression of mature MD protein. Alternatively, when the yeast is Kluveromyces and the signal-peptide/De/5 gene is under the control of a Lactase promoter, addition of galactose to the growth medium will provide for expression of mature MD protein.

Once the transformed yeast culture has been incubated under culture conditions that provide for expression of mature MD protein or peptide for a sufficient period of time, the mature MD molecule can be isolated from the culture. A sufficient period of time can be determined by periodically harvesting portions or aliquots of the culture and assaying for the presence of MD protein or peptide. Analytical assays such as SDS-PAGE with protein staining, Western blot analysis, or any immunodetection method (e.g., such as an ELISA) can be used to monitor MD production. For example, incubation in the presence of methanol for between 1 to 8 days is sufficient to provide for expression of mature MD protein from the AOX1 promoter in Pichia.

Isolation of the MD protein or peptide from the culture can be partial or complete. For MD expression vectors where a yeast signal peptide is operably linked to the sequence encoding the mature MD protein, the mature MD protein can be recovered from the yeast cell culture medium. Yeast cell culture medium that contains the mature MD protein can be separated from the yeast cells by centrifugation or filtration, thus effecting isolation of mature MD protein. Yeast cell culture medium that contains the mature MD protein can be further processed by any combination of dialysis and/or concentration techniques (e.g., precipitation, lyophilization, filtration) to produce a composition containing the MD protein. Production of MD protein can also comprise additional purification steps that result in either a partially or completely pure preparation of the MD protein. To effect such purification, filtration size-exclusion membranes can be used. Alternatively, various types of chromatographic techniques such as size exclusion chromatography, ion-exchange chromatography, or affinity chromatography can be used to produce a partially or completely pure preparation of the MD protein.

Combinations of various isolation techniques can also be employed to produce the mature MD protein. For example, the cell culture medium can be separated from the cells by centrifugation and dialyzed or adjusted. In certain embodiments, a buffer for dialysis or adjustment is a 25 mM sodium acetate buffer at about pH4.5-pH6.0. This dialysate is then subjected to ion-exchange chromatography. For example, a cation-exchange resin such as CM-Sephadex C-25 equilibrated with a 25 mM sodium acetate buffer at about pH6.0 can be used. MD protein bound to the cation exchange resin is washed and then eluted. For example, the aforementioned column is washed with 25 mM sodium acetate buffer at about pH6.0 and subsequently eluted in 1M NaCl, 50mM Tris, pH7.6. Fractions containing the defensin protein are identified by an assay or by UV absorbance and then concentrated by a size-cutoff filtration membrane. The concentrated MD protein is then dialyzed to obtain an essentially or substantially pure MD protein in a buffer. Buffers include, but are not limited to, buffers such as 10 mM Tris, pH 7.6.

Also provided are antifungal compositions for agricultural or pharmaceutical use comprising either an antifungal plant, or antifungal human or veterinary, pathogenic fungus inhibitory amount (“antifungal effective amount”) of one or more the present isolated, purified antifungal MD proteins or peptides, or biologically functional equivalents thereof. Such compositions can comprise one, or any combination of, MD proteins or peptides disclosed herein, and an agriculturally, pharmaceutically, or veterinary-practicably acceptable carrier, diluent, or excipient. As indicated below, other components relevant in agricultural and therapeutic contexts can be included in such compositions as well. The antifungal compositions can be used for inhibiting the growth of, or killing, MD protein- or peptide-susceptible pathogenic fungi associated with plant, human or animal fungal infections. Such antifungal compositions can be formulated for topical administration, and applied topically to either plants, the plant environment (including soil), or humans or animals.

Agricultural compositions comprising any of the present MD molecules alone, or in any combination, can be formulated as described in, for example, Winnacker-Kuchler (1986) Chemical Technology, Fourth Edition, Volume 7, Hanser Verlag, Munich; van Falkenberg (1972-1973) Pesticide Formulations, Second Edition, Marcel Dekker, N.Y.; and K. Martens (1979) Spray Drying Handbook, Third Edition, G. Goodwin, Ltd., London. Formulation aids, such as carriers, inert materials, surfactants, solvents, and other additives are also well known in the art, and are described, for example, in Watkins, Handbook of Insecticide Dust Diluents and Carriers, Second Edition, Darland Books, Caldwell, N.J., and Winnacker-Kuchler (1986) Chemical Technology, Fourth Edition, Volume 7, Hanser Verlag, Munich. Using these formulations, it is also possible to prepare mixtures of the present MD proteins and peptides with other pesticidally active substances, fertilizers, and/or growth regulators, etc., in the form of finished formulations or tank mixes.

Whether alone or in combination with other active agents, the present antifungal MD proteins and peptides can be applied at a concentration in the range of from about 0.1 μg ml to about 100 mg ml, or from about 5 μg ml to about 5 mg ml, at a pH in the range of from about 3.0 to about 9.0. Such compositions can be buffered using, for example, phosphate buffers between about 1 mM and 1 M, about 10 mM to about 100 mM, or about 15 mM to about 50 mM. In the case of low buffer concentrations, a salt can be added to increase the ionic strength. In certain embodiments, NaCl in the range of from about 1 mM to about 1 M, or about 10 mM to about 100 mM, can be added.

Numerous conventional fungal antibiotics and chemical fungicides with which the present MD proteins and peptides can be combined are known in the art, and are described in Worthington and Walker (1983) The Pesticide Manual, Seventh Edition, British Crop Protection Council. These include, for example, polyoxines, nikkomycines, carboxy amides, aromatic carbohydrates, carboxines, morpholines, inhibitors of sterol biosynthesis, and organophosphorous compounds. Other active ingredients which can be formulated in combination with the present antifungal proteins and peptides include, for example, insecticides, attractants, sterilizing agents, acaricides, nematocides, and herbicides. U.S. Pat. No. 5,421,839, which is incorporated herein by reference in its entirety, contains a comprehensive summary of the many active agents with which substances such as the present antifungal MD proteins and peptides can be formulated.

Agriculturally useful antifungal compositions encompassed herein also include those in the form of host cells, such as bacterial and fungal cells, capable of the producing the MD proteins and peptides, and which can colonize plants, including roots, shoots, leaves, or other parts of plants. The term “plant-colonizing microorganism” is used herein to refer to a microorganism that is capable of colonizing the any part of the plant itself and/or the plant environment, including, and which can express the present MD antifungal proteins and peptides in the plant and/or the plant environment. A plant colonizing micro-organism is one that can exist in symbiotic or non-detrimental relationship with a plant in the plant environment. U.S. Pat. No. 5,229,112, which is incorporated herein by reference in its entirety, discloses a variety of plant-colonizing microorganisms that can be engineered to express antifungal proteins, and methods of use thereof, applicable to the MD antifungal proteins and peptides disclosed herein. Plant-colonizing microorganisms expressing the presently disclosed MD antifungal proteins and peptides useful in inhibiting fungal growth in plants include, but are not limited to, bacteria selected from the group consisting of Bacillus spp. including but not limited to Bacillus thuringiensis, Bacillus israelensis, and Bacillus subtilis, Candidatus Liberibacter asiaticus; Pseudomonas spp.; Arthrobacter spp., Azospyrillum spp., Clavibacter spp., Escherichia spp.; Agrobacterium spp., for example A. radiobacter, Rhizobium spp., Erwinia spp. Azotobacter spp., Azospirillum spp., Klebsiella spp., Alcaligenes spp., Rhizobacterium spp., Xanthomonas spp., Ralstonia spp. and Flavobacterium spp., In certain embodiments, the microorganism is a yeast selected from the group consisting of Saccharomyces cerevisiae, Pichia pastoris, and Pichia methanolica. In certain embodiments, the plant colonizing microorganism can be an endophytic bacteria or fungi.

When applying the present MD molecules to the rhizosphere, rhizosphere-colonizing bacteria from the genus Pseudomonas are particularly useful, especially the fluorescent pseudomonads, e.g., Pseudomonas fluorescens, which is especially competitive in the plant rhizosphere and in colonizing the surface of the plant roots in large numbers. Examples of suitable phylloplane (leaf) colonizing bacteria are P. putida, P. syringae, and Erwinia species.

The antifungal plant-colonizing microorganisms that can express MD can be applied directly to the plant, e.g., to the surface of leaves, buds, roots, shoots, floral parts, seeds, etc., or to the soil. When used as a seed coating, the plant-colonizing microorganisms that can express MD are applied to the plant seed prior to planting. The determination of an antifungal effective amount of plant-colonizing microorganisms used for a particular plant can be empirically determined, and will depend on such factors as the plant species, the fungal pathogen, method of planting, and the soil type, (e.g., pH, organic matter content, moisture content). At least one, 10 or 100 plant-colonizing microorganism(s) containing DNA encoding the MD antifungal proteins and peptides disclosed herein is sufficient to control fungal pathogens because it or they can grow into a colony of clones of sufficient number to express antifungal amounts of the MD. However, in practice, due to varying environmental factors which can affect the survival and propagation of the microorganism, a sufficient number of plant colonizing microorganisms should be provided in the seed, plant or plant environment (e.g., roots or foliage) to assure survival and/or proliferation. For example, application of 103 to 1010 bacteria or yeasts per seed can be sufficient to insure colonization on the surface of the roots by the microorganism. In certain embodiments, it is sufficient to dose the plant or plant environment with enough bacteria or other plant-colonizing microorganism to maintain a population that expresses 100 to 250 nanograms of the MD per plant. For example, 105 to 108 bacteria per square centimeter of plant surface can be adequate to control fungal infection. In certain embodiments, at least about 5 or 10 nanograms to about 100, 200, 500, or 1,000 nanograms, of a MD protein can be sufficient to control fungal damage to plants.

Compositions containing the plant colonizing microorganisms that express the MD can be prepared by formulating the biologically active microorganism with adjuvants, diluents, carriers, etc., to provide compositions in the form of finely-divided particulate solids, granules, pellets, wettable powders, dusts, aqueous suspensions, dispersions, or emulsions. Illustrative of suitable carrier vehicles are: solvents, e.g., water or organic solvents, and finely divided solids, e.g., kaolin, chalk, calcium carbonate, talc, silicates, and gypsum. In certain embodiments, plant colonizing microorganisms that express the MD can also be in encapsulated form, e.g., the plant-colonizing microorganisms can be encapsulated within shell walls of polymer, gelatin, lipid, and the like. Other formulation aids such as, for example, emulsifiers, dispersants, surfactants, wetting agents, anti-foam agents, and anti-freeze agents, can be incorporated into the antifungal compositions, especially if such compositions will be stored for any period of time prior to use.

In addition to the plant-colonizing microorganisms that express MD, the compositions provided herein can additionally contain other known biologically active agents, such as, for example, a fungicide, herbicide, or insecticide. Also, two or more plant-colonizing microorganisms that express either a different or the same MD can be combined.

The application of antifungal compositions containing the genetically engineered plant-colonizing microorganisms that can express MD as the active agent can be carried out by conventional techniques utilizing, for example, spreaders, power dusters, boom and hand sprayers, spry dusters, and granular applicators.

The compositions provided herein can be applied in an antifungal effective amount, which will vary depending on such factors as, for example, the specific fungal pathogen to be controlled, the specific plant (and plant part or soil) to be treated, and the method of applying the compositions that comprise MD.

MD proteins and peptides and biologically functional equivalents, as well transgenic or genetically edited plants or microorganisms expressing those proteins, can be used to inhibit the growth of a wide variety of susceptible fungi in plants. In certain embodiments, growth of fungi in the following genera or species can be inhibited: Alternaria (e.g., Alternaria brassicola; Alternaria solani); Ascochyta (e.g., Ascochyta pisi); Aspergillus (e.g., Aspergillus flavus; Aspergillus fumigatus); Botrytis (e.g., Botrytis cinerea); Cercospora (e.g., Cercospora kikuchii; Cercospora zeae-maydis); Colletotrichum (e.g., Colletotrichum lindemuthianum); Diplodia (e.g., Diplodia maydis); Erysiphe (e.g., Erysiphe graminis f.sp. graminis; Erysiphe graminis fsp. hordei); Fusarium (e.g., Fusarium nivale; Fusarium oxysporum; Fusarium graminearum; Fusarium culmorum; Fusarium solani; Fusarium moniliforme; Fusarium roseum); Gaeumanomyces (e.g., Gaeumanomyces graminis fsp. tritici); Helminthosporium (e.g., Helminthosporium turcicum; Helminthosporium carbonum; Helminthosporium maydis); Macrophomina (e.g., Macrophomina phaseolina; Maganaporthe grisea); Nectria (e.g., Nectria heamatococca); Peronospora (e.g., Peronospora manshurica; Peronospora tabacina); Phakopsora (e.g., Phakopsora pachyrhizi); Phoma (e.g., Phoma betae); Phymatotrichum (e.g., Phymatotrichum omnivorum); Phytophthora (e.g., Phytophthora cinnamomi; Phytophthora cactorum; Phytophthora phaseoli; Phytophthora parasitica; Phytophthora citrophthora; Phytophthora megasperma fsp. sojae; Phytophthora infestans); Plasmopara (e.g., Plasmopara viticola); Podosphaera (e.g., Podosphaera leucotricha); Puccinia (e.g., Puccinia sorghi; Puccinia striiformis; Puccinia graminis f.sp. tritici; Puccinia asparagi; Puccinia recondita; Puccinia arachidis); Pythium (e.g., Pythium aphanidermatum; Pythium ultimum); Pyrenophora (e.g., Pyrenophora tritici-repentens); Pyricularia (e.g., Pyricularia oryzae); Rhizoctonia (e.g., Rhizoctonia solani; Rhizoctonia cerealis); Scerotium (e.g., Scerotium rolfsii); Sclerotinia (e.g., Sclerotinia sclerotiorum); Septoria (e.g., Septoria lycopersici; Septoria glycines; Septoria nodorum; Septoria tritici); Thielaviopsis (e.g., Thielaviopsis basicola); Uncinula (e.g., Uncinula necator); Venturia (e.g., Venturia inaequalis); and Verticillium (e.g., Verticillium dahliae; Verticillium alboatrum).

Pharmaceutical or veterinary compositions that comprise an antifungal effective amount of MD proteins, peptides, or biologically functional equivalents thereof and a pharmaceutically acceptable carrier are also provided. Such pharmaceutical or veterinary compositions can be used for inhibiting the growth of, or killing, susceptible pathogenic fungi that infect humans or animals, i.e., treating such fungal infections by administering to a patient or other subject in need thereof. In certain embodiments, compositions comprising MD proteins and peptides, and biologically functional equivalents thereof, can be formulated by methods such as those described in Remington: The Science and Practice of Pharmacy (2005), 21st Edition, University of the Sciences in Philadelphia, Lippincott Williams & Wilkins. In certain embodiments, the compositions can contain MD proteins and peptides, and various combinations thereof, at concentrations in the range of from about 0.1 μg ml to about 100 mg ml, or about 5 μg per ml to about 5 mg per ml, at a pH in the range of from about 3.0 to about 9.0. Such compositions can be buffered using, for example, phosphate buffers at a concentration of about 1 mM to about 1 M, about 10 mM to about 100 mM, or about 15 mM to 50 mM. In the case of low buffer concentrations, a salt can be added to increase the ionic strength. In certain embodiments, NaCl in the range of about 1 mM to about 1 M, or about 10 mM to about 100 mM, can be added.

The MD proteins and peptides can be formulated alone, in any combination with one another, and either of these can additionally be formulated in combination with other conventional antifungal therapeutic compounds such as, by way of non-limiting example, polyene antifungals; imidazole, triazole, and thiazole antifungals; allylamines; and echinocandins that are routinely used in human and veterinary medicine.

Administration of the compositions that comprise MD to a human or animal subject in need thereof can be accomplished via a variety of conventional routes that include, but are not limited to, topical application.

### EXAMPLES

### Example 1

### Description of MtDef5FL Gene and Encoded MtDef5FL1, MtDef5FL2, and MtDef5FL3 Proteins

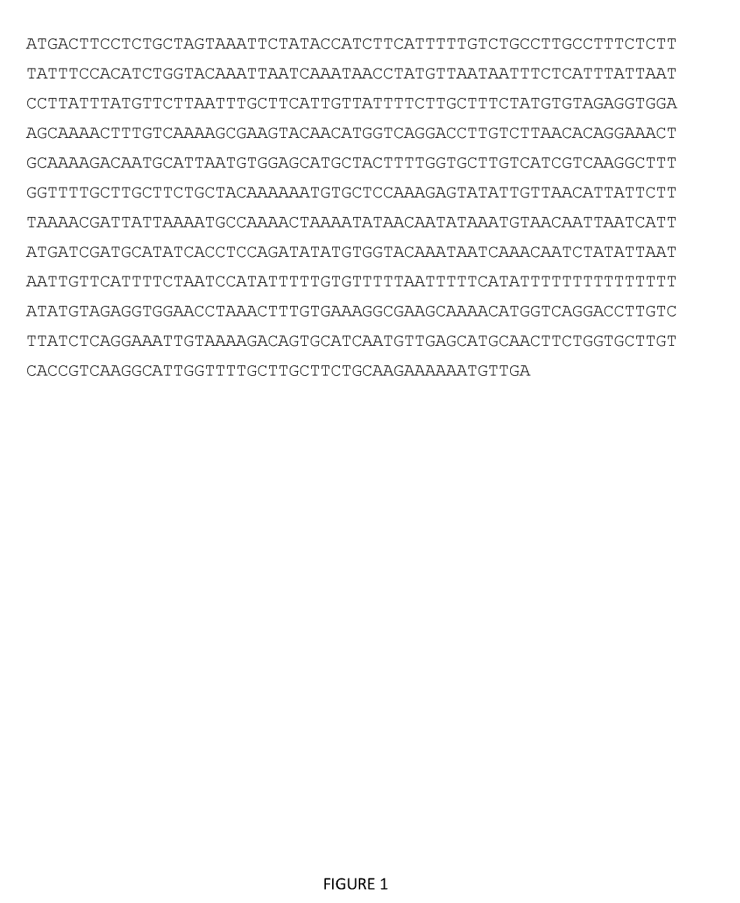

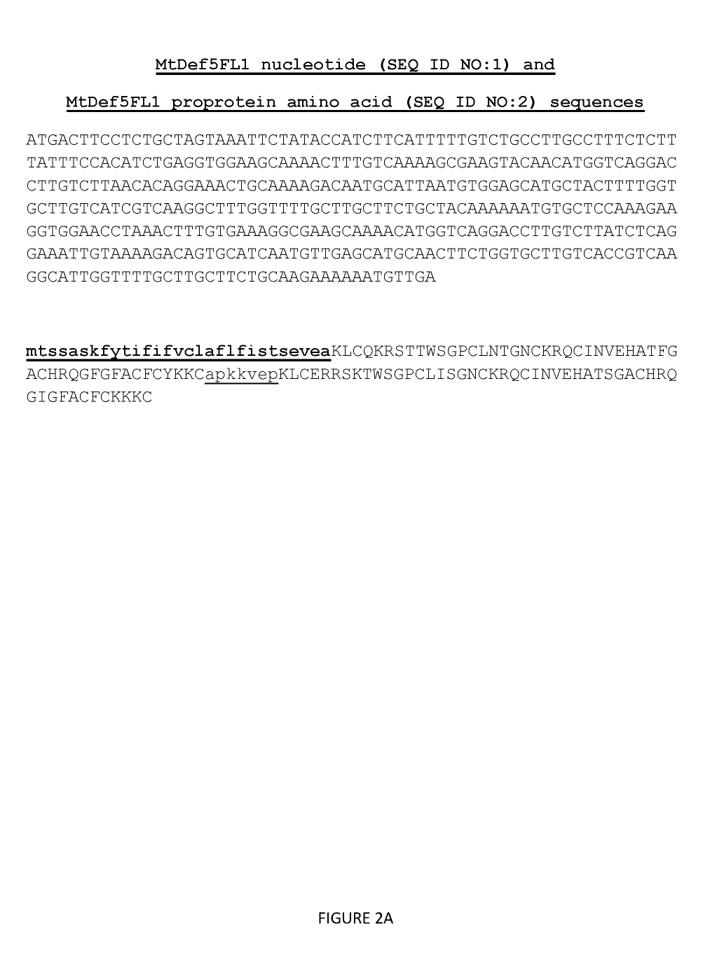

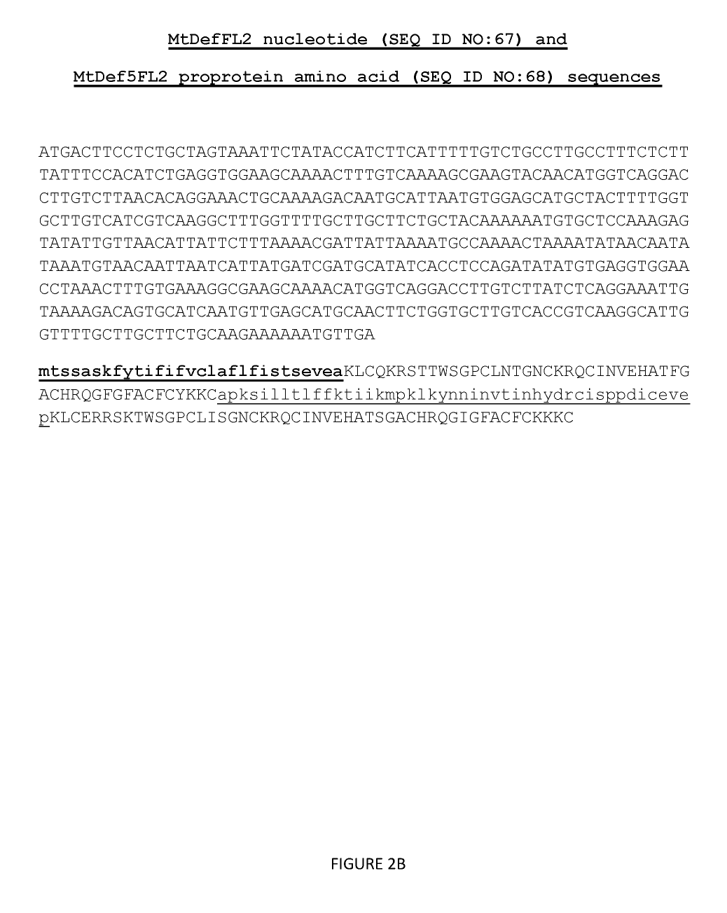

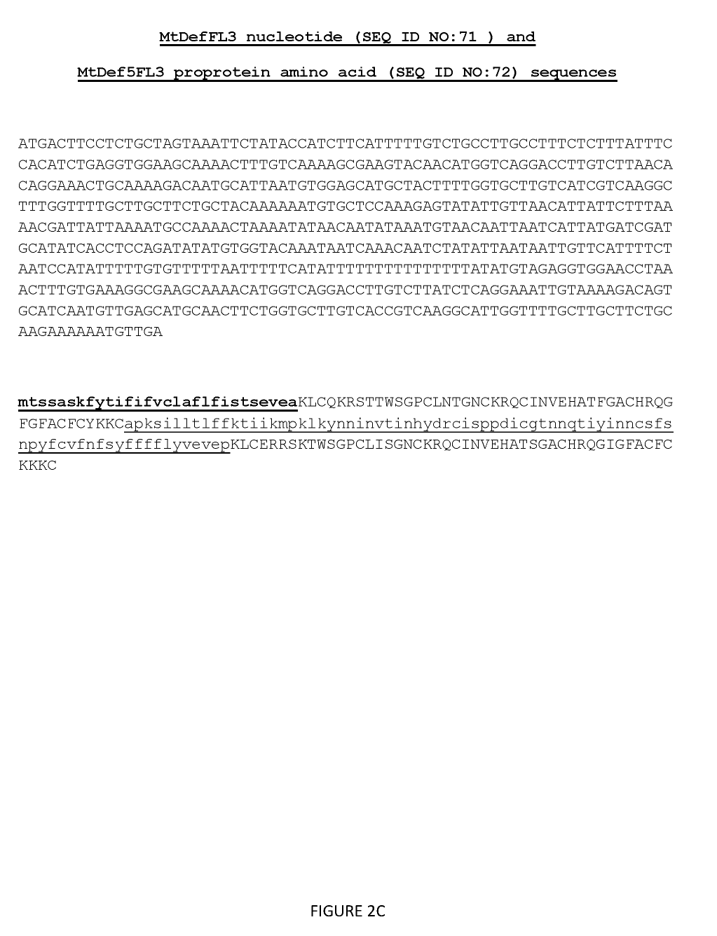

The Medicago truncatula genome contains 63 predicted genes encoding small cysteine-rich defensins that contain four disulfide bonds. The nucleotide sequence of one of these genes (Medtr8g012775 or MtDef5FL1; SEQ ID NO:1) is shown in FIG. 1. This gene is predicted to be alternatively spliced giving rise to three proteins (FIG. 2A, B, C). The amino acid sequence of each of the three proteins however contains two defensin peptides of 50-amino acid each connected by a linker peptide sequence of different length. The homology based modeling of each defensin peptide predicts one α-helix and three antiparallel β-strands connected by four disulfide bonds. Each protein also contains a secretory signal peptide of 28-amino acids which enables the mature protein to be secreted to the apoplast.

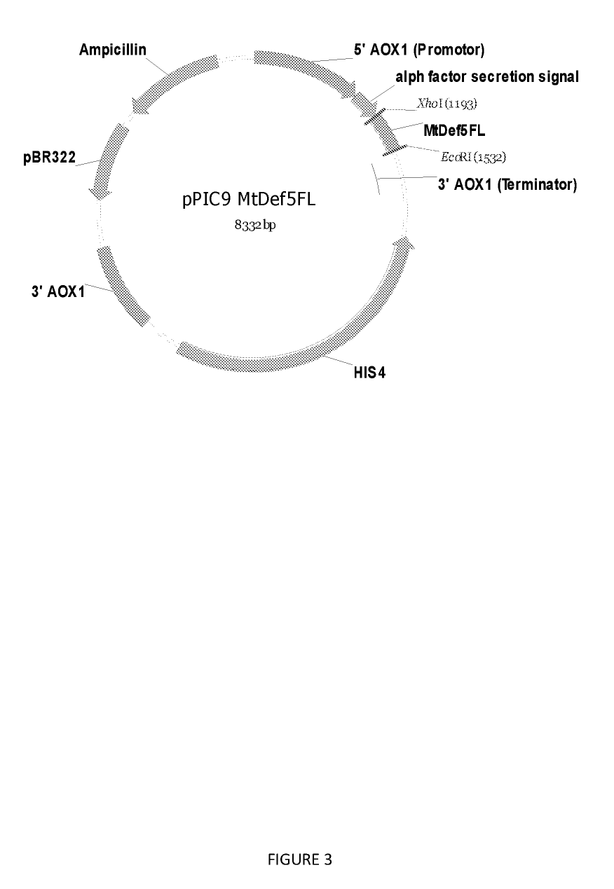

The DNA sequence encoding the mature defensin sequence of MtDef5FL1 (Medtr8g012775.1; SEQ ID NO:63) was codon-optimized by GenScript (Piscataway, N.J.) for P. pastoris expression using OptimumGene™ Algorithm (SEQ ID NO:64). A sequence encoding the XhoI restriction endonuclease and the KEX2 protease site (CTCGAGAAAAGA; SEQ ID NO:65) was added upstream of the MtDef5FL1 mature coding sequence. Two stop codons, along with an EcoRI restriction enzyme site (TAATGAGAATTC; SEQ ID NO:66) were added downstream of the MtDef5FL1 mature coding sequence. The algorithm optimized a variety of parameters that were critical to the efficiency of MtDef5FL1 gene expression, including but not limited to codon usage bias, GC content, CpG dinucleotides content, mRNA secondary structure, cryptic splicing sites, premature PolyA sites, internal chi sites and ribosomal binding sites, negative CpG islands, RNA instability motif, repeat sequences and, restriction sites that can interfere with cloning. The codon-optimized synthetic MtDef5FL1 gene was cloned into EcoRV digested pUC57. The synthetic gene was purchased from GenScript (Piscataway, NJ). The resultant pUC57/MtDef5FL1 plasmid was transformed into One Shot® Top10 competent E. coli cells (Invitrogen) and the recombinant cells were selected on carbenicillin-containing LB agar plates. The resulting recombinant plasmid was digested with Xhol and EcoRI, and ligated further into the Xhol and EcoRI digested pPIC9. The resulting vector (FIG. 3) contained a nucleic acid sequence encoding the mature MtDef5FL1 defensin sequence (SEQ ID NO:7) fused in frame with the a-factor secretion signal sequence. This nucleic acid sequence was operably linked to the P. pastoris alcohol oxidase promotor. After transformation into Top10 competent E. coli cells, the pPIC9-MtDef5FL1 plasmid was isolated and then linearized by digestion with SalI restriction enzyme and transformed into competent cells of P. pastoris GS115 by electroporation (Gene Pulser® II, Bio-Rad, USA), according to the manufacturer's instructions (Pichia Expression Kit®, Invitrogen). His+ transformants were selected by plating on minimal dextrose and minimal methanol plates.

P. pastoris colony containing the pPIC9MtDef5FL construct was inoculated into buffered minimal glycerol (BMG) media and after overnight growth transferred into buffered minimal methanol (BMM) media. The promoter driving the expression of MtDef5FL was induced with methanol every 24 hour, according to the manufacturer's directions (Invitrogen). The cultures were grown for 7 days at 28o C, and cells were removed by centrifugation at 4000 rpm 5 min and retained the supernatant. The pH of the supernatant was adjusted to 6.0 and CM-Sephadex C-25 cation-exchange resin (Amersham Biosciences, Piscataway, N.J.) was added to bind protein and incubated in incubator at 4° C. and 110 rpm overnight. The slurry was poured through MiraCloth® in a Buchner funnel and resin was collected and packed into FPLC (Fast Protein Liquid Chromatography column). Resin was extensively washed with binding buffer (25 mM sodium acetate, pH 6), and the bound protein was then eluted in 1 M NaCl, 50 mM Tris, pH 7.6. FPLC fractions were collected and concentrated at 4° C. in Amicon Stirred Cell concentrator with 3000 Molecular Weight Cut Off (MWCO) membrane and dialyzed in 1000 MWCO tubing against 50 mM Tris pH 7.6. The dialysate was filter sterilized through 0.2 μm filter and lyophilized. Lyophilized protein was resuspended in nuclease free water and the protein concentration was determined by bicinchonic acid (BCA) assay using the micro plate protocol provided by the manufacturer (Pierce, Rockford, Ill.). Purity and size of the protein isolate were assessed using 15% SDS-PAGE gel (FIG. 4).

The antifungal activity of MtDef5FL was measured in an in vitro assay using 96-well microtiter plates. Fifty microliters (μL) of each protein dilution were added to each well of the microtiter plate containing 50 μL of spore suspension. F. graminearum PH-1, Fusarium virguliforme and Neurospora crassa spores were harvested from carboxymethyl cellulose liquid medium, potato dextrose agar (Difco, Sparks, Md.) and Vogel's (Vogel, 1964) agar medium, respectively. For conidial suspension, Vogel's liquid medium was used for N. crassa and 2× synthetic fungal medium (SFM) without calcium was used for F. graminearum and F. virguliforme. To monitor the early visible phenotypic effects of MtDef5FL on conidial germination and growth of fungal hyphae (at 14-16 hours after treatment with MtDef5FL), bright-field images were taken using the transmitted light channel in a Leica SP8-X confocal microscope. The quantitative fungal growth inhibition by MtDef5FL was estimated by measuring the absorbance at 595 nm using Spectramax M2 spectrophotometer (Molecular Devices, Sunnyvale, Calif.) (Broekaert et al., 1990).

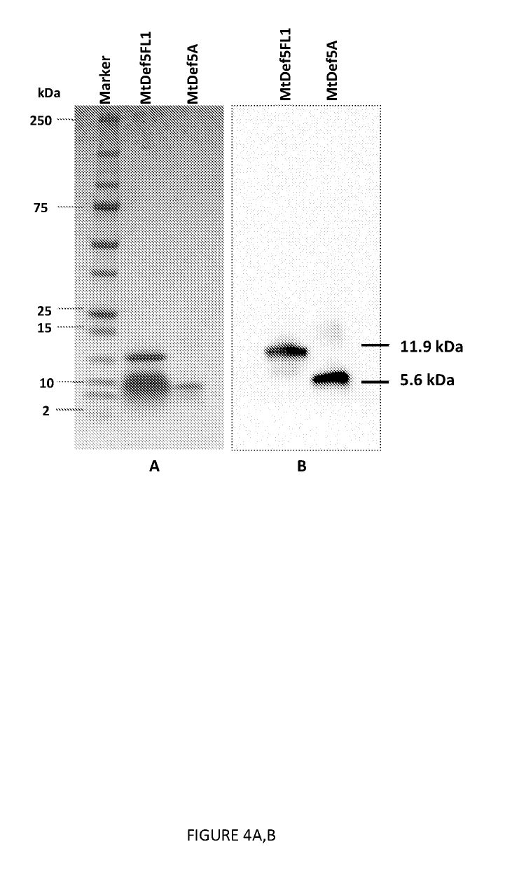

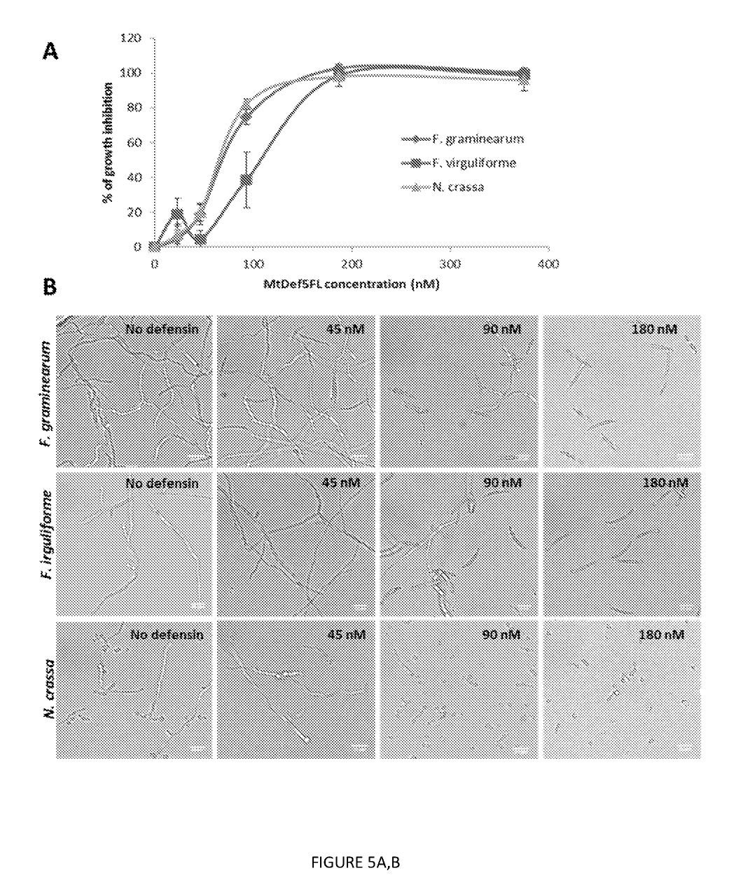

MtDef5FL1 displayed phenomenal antifungal activity against all three fungi. The IC50 value of MtDef5FL1 against F. graminearum, F. virguliforme and N. crassa was 0.090 μM, 0.090 μM and 0.090 μM, respectively (FIG. 5A). It was remarkably potent against F. graminearum, F. virguliforme and N. crassa with an IC100 value of 0.18 μM (FIG. 5A). Microscopic observations after 14-16 hr incubation of F. graminearum, F. virguliforme and N. crassa conidia indicated that MtDef5FL1 exhibited antifungal activity at concentrations as low as 0.090 μM and inhibited conidial germination completely (100% growth inhibition) at 0.18 μM (FIG. 5B). Thus, MtDef5FL1 is a potent antifungal protein with strong potential as an antifungal agent in transgenic crops.

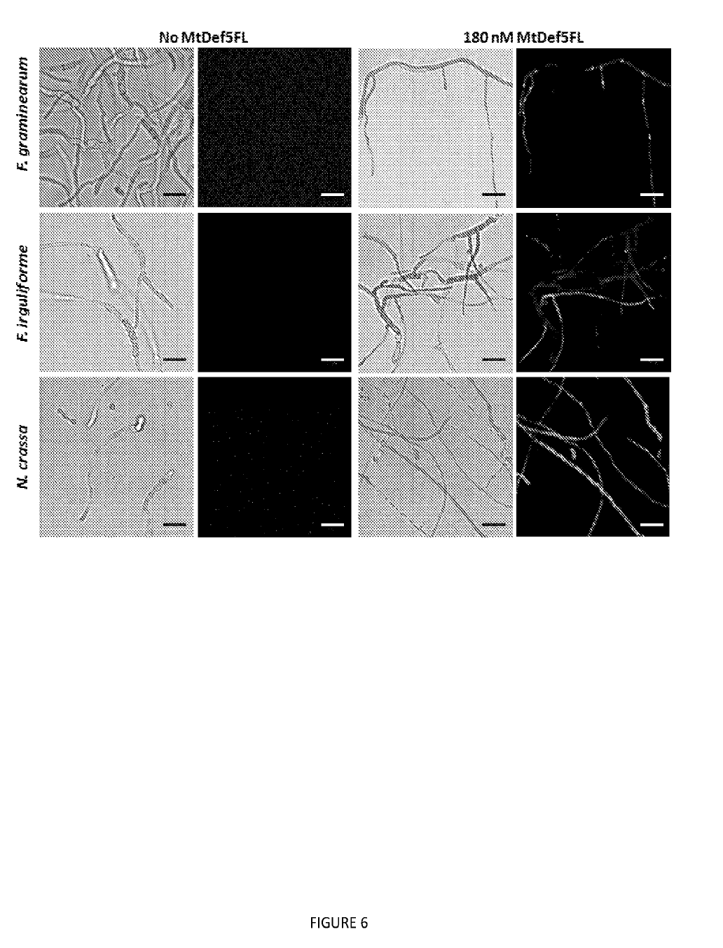

MtDefS was examined for its ability to permeabilize F. graminearum, F. virguliforme and N. crassa plasma membrane using the fluorometric SYTOX Green (SG) dye which is only taken up by cells with a compromised plasma membrane. Permeabilization was measured by confocal microscopy following SG uptake assay. SG uptake was monitored under microscope after 2 hrs of incubation at 0.180 μM (IC100) of MtDefS along with 0.5 μm SG. SG uptake was observed with MtDef5FL in all three fungi (FIG. 6). These results indicate that MtDef5FL1 disrupts the plasma membrane of F. graminearum, F. virguliforme and N. crassa, and therefore permeabilizes the fungal cell.

### Example 2

### Phospholipid Binding by MtDefS Defensin Peptide A and MtDef5FL1

To test the binding properties of MtDefS peptide A (MtDefS Peptide A; SEQ ID NO:4) and MtDef5FL1 (SEQ ID NO:7), a protein-lipid overlay experiment was performed using PIP Strips™ (2×6 cm nitrocellulose membranes) that are spotted with 100 pmol of various biologically active lipids (Echelon Biosciences, Salt Lake City, Utah). Briefly, lipid strips were blocked with 10 mL of blocking buffer, PBS-T/3% fat free BSA for 12-16 h at 4° C. with gentle agitation. The blocking buffer was discarded and the lipid strips were incubated with 5 mL of PBS-T/3% fat free BSA containing 1 μg/ml of MtDef5 peptide A or MtDef5FL1 for 60 min at 4° C. After hybridization, protein solution was discarded and the strips were then washed with PBS-T three times with gentle agitation for 20 minutes each at room temperature. The protein-lipid interactions were detected by subsequently incubating lipid strips with rabbit anti-MtDef5 peptide A derived synthetic peptide (1 μg/ml) diluted in 5 mL of blocking buffer with gentle agitation for 60 min at 4° C. The lipid strips were washed with PBS-T two times with gentle agitation, for 15 minutes each wash, at room temperature. HRP-conjugated Goat Anti-Rabbit IgG secondary antibody (Thermo Scientific, Cat no: SA 1-9510) diluted 1:4000 in blocking buffer was added to the lipid strips and incubated for 60 min at 4° C. After two washes with PBS-T, chemiluminescence was detected using SuperSignal® West Femto Maximum Sensitivity substrate kit (Thermo Scientific, Cat no: 34094) following the manufacturer's protocol.

To investigate the spectrum of potential lipid signals interacting with MtDef5A or MtDef5FL1, protein-lipid overlay assay was performed with various biologically active lipids immobilized on a nitrocellulose membrane (FIG. 9). Using this assay, we have determined that MtDef5FL1 strongly binds to phosphatidylinositol 3-phosphate (PI3P), phosphatidylinositol 4-phosphate (PI4P) and phosphatidylinositol 5-phosphate (PISP). Although the MtDef5FL1 showed much higher affinity for phosphatidylinositol monophosphates, it also binds to a lesser extent to other functionally important membrane lipids such as phosphatidylinositol 4,5-bisphosphate (PI4,5P2), phosphatidylinositol 3,5-bisphosphate (PI3,5P2) and phosphatidylinositol 3,4-bisphosphate (PI3,4P2). MtDef5 peptide A also binds to PIPs and PIP2. However, MtDef5 peptide A binds to PI3P, PI4P and PISP with much less affinity than MtDef5FL1. Moreover, MtDef5 peptide A fails to bind PS.

The presence of two MtDef5 defensin peptides connected by a short peptide sequence on MtDef5FL1 significantly increases its binding affinity to PI3P, PI4P and PISP. In addition, a new lipid binding specificity, i.e., binding to PS, is generated.

### Example 3

### Expression of MtDef5FL1 in Transgenic Arabidopsis

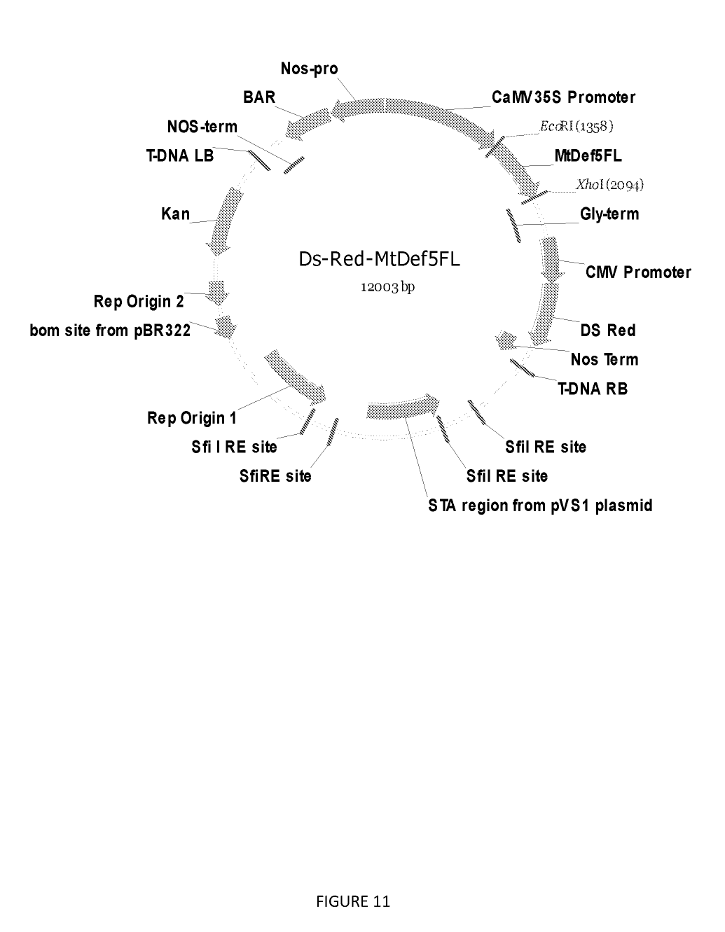

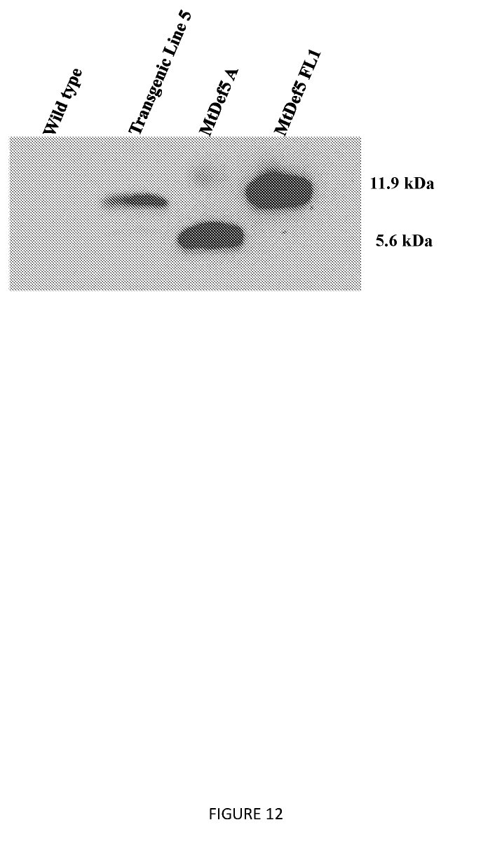

For expression of MtDef5FL1 in Arabidopsis thaliana, the 742 bp DNA sequence (SEQ ID NO: 1) encoding the signal peptide and the two defensin peptides A & B of MtDef5FL1 was synthesized by GenScript (Piscataway, N.J.). The EcoRI and Xhol restriction endonuclease sites were introduced at the 5′ and 3′ ends of the MtDef5FL nucleotide sequence. The synthetic MtDef5FL gene was cloned into EcoRV digested pUC57. The resulting pUC57/MtDef5FL plasmid was transformed into One Shot® Top10 competent E. coli cells (Invitrogen) and the recombinant cells were selected on carbenicillin-containing LB agar plates. The recombinant plasmid was digested with EcoRI and Xhol, and ligated into the Xhol and EcoRI digested vector Ds-Red. The Ds-Red/MtDef5FL plasmid thus generated (FIG. 11) was transformed into One Shot® Top10 competent E. coli cells (Invitrogen) and the recombinant cells were selected on kanamycin-containing LB agar plates. The vector Ds-Red, containing the MtDef5FL gene driven by the CaMV 35S promoter together with Ds-red gene driven by the CaMV 35S promoter, was transferred into Agrobacterium tumefaciens strain GV301. Arabidopsis thaliana Col-0 plants were transformed using the floral dip method (Kaur et al., Molecular Plant Pathology 13, 1032-1046, 2012). Putative T1 transformants were selected by screening for Ds-red fluorescence in seeds. T2 seeds showing 3 fluorescent: 1 non-fluorescent segregation ratio of Ds-Red positive were planted to obtain homozygous lines (T3).

To determine MtDef5FL1 expression in T2 generated transgenic Arabidopsis lines, the immunoblot analysis was performed using a primary antibody raised against MtDef5A synthetic peptide. Leaf tissue (100-200 mg) was ground to a fine powder in liquid nitrogen and the homogenate was suspended in 200 μl of ice-cold extraction buffer containing 250 mM Tris-HCl ), pH 7.5, 2.5 mM EDTA, 0.1% ascorbic acid, 1 mM PMSF. The tissue suspension was centrifuged at 12000 rpm for 10 min at 4° C. The supernatant was collected and the protein was quantified using the Bradford assay. Protein (20 μg) was diluted with an equivalent volume of 2× Laemmli sample buffer and reducing agent (100 mM DTT). The samples were heated at 95° C. for 5 min and loaded on 4-20% SDS-PAGE gel. Following electrophoresis, samples were transferred to a nitrocellulose membrane and immunoblotted using the anti-MtDef5 polyclonal antibody (1 μg/ml) and the HRP-conjugated Goat Anti-Rabbit IgG secondary antibody (1:4000). Signal was detected using the SuperSignal West Pico Chemiluminescent Substrate Kit (Thermo-Scientific) following the manufacturer's protocol. The Western blot analysis of the total protein extracted from the leaf tissue of the transgenicArabidopsisline showed expression of the predicted 11.9 kDa MtDef5FL1 protein.

### Example 4

### Characterization of a Leersia perrieri defensin protein

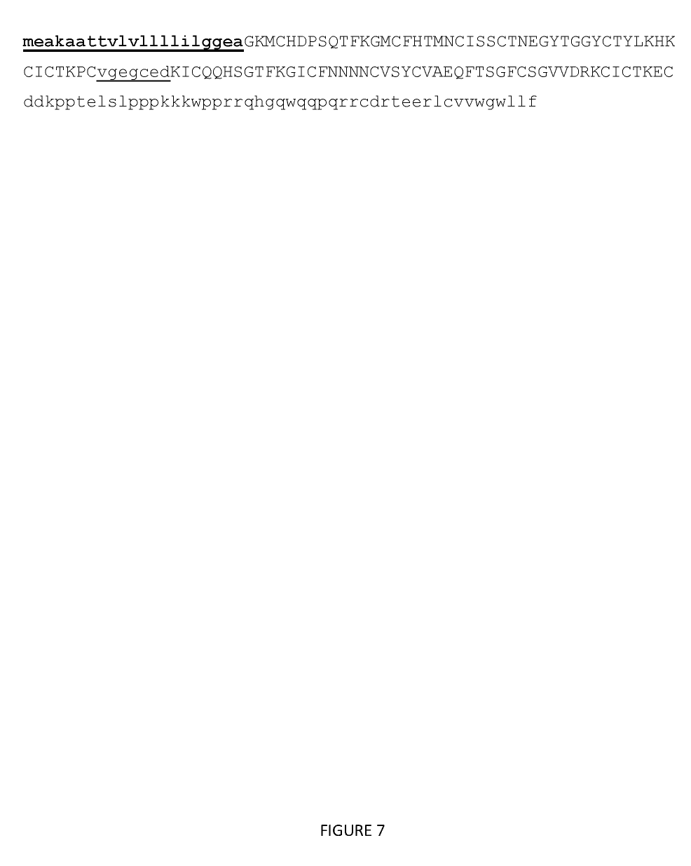

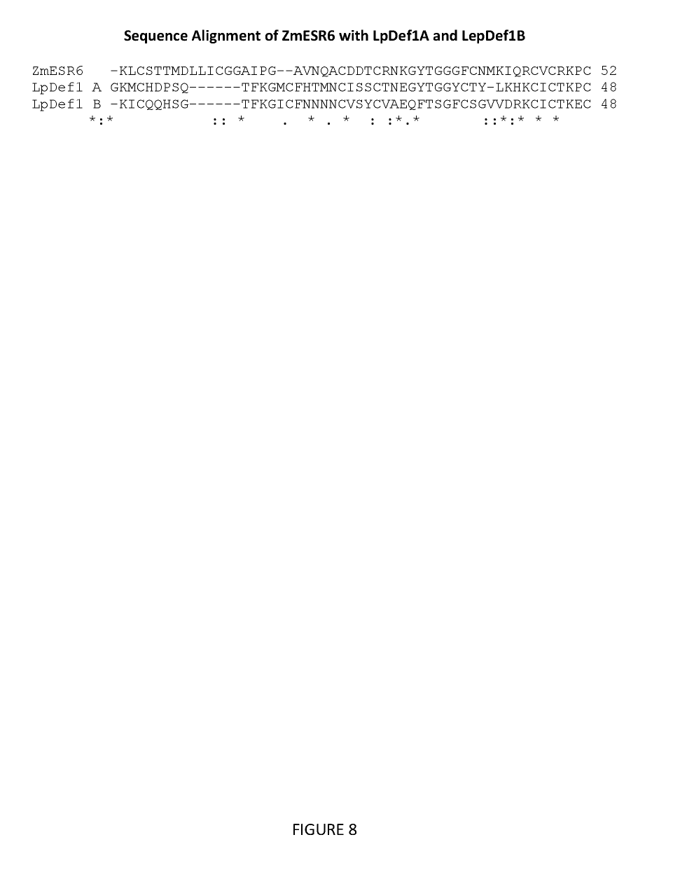

The mature peptide coding sequence of the maize defensin ZmESR-6 (Uniprot ID: Q5CC32 in the Uniprot protein database on the World Wide Web at uniprot.org); SEQ ID NO:62) from Zea mays was used as a BLASTP (UniProt Database) query to identify a novel defensin from Leersia perrieri. A search using the ZmESR-6 as a query has revealed one novel defensin gene that encodes a protein with 173 amino acids, named LpDef1 (Uniprot ID: AOAOD9VCR3; SEQ ID NO:56). The presence of a signal peptide was predicted using signalP and the results showed that the first 21 amino acids of LpDef1 defensin peptide encode an N-terminal secretory signal peptide followed by a 152 amino acid peptide (FIG. 7). Notwithstanding the conserved pattern shared by all plant classical defensins, subsequent examination has confirmed that this dimeric protein consists of two defensin peptides connected by a peptide linker sequence. The linker peptide (SEQ ID NO:60) contains diacidic residues. In certain contexts, diacidic residues in peptides can be cleaved by plant endoproteinases. A peptides with the ZmESR-6 defensin peptide contained both highly conserved cysteine residues and γ-core structural motifs (CXCX3-9C) between mature peptide amino acid sequences in each peptide (FIG. 8). Analysis of the deduced mature amino acid sequences showed that both defensin peptide A and defensin peptide B consist of 48 amino acid residues each (FIG. 7; SEQ ID NO:56, 57, 58, 59, 60, and 61).

### Example 5

### Biological Sequences and Associated SEQ ID NO

TABLE 2
Biological sequences

| | SOURCE | | | |
|---|---|---|---|---|
| | AND GENE | | | |
| | NAME | | | |
| | (Defensin | | | |
| | Name, | | | |
| | Uniprot | | | |
| | Accession, | | | |
| | U.S. Pat. | | | |
| | No., and/or | | | |
| SEQ | U.S. patent | | | |
| ID | application | | | |
| NO | Pub. No.) | TYPE | PEPTIDE¹ | SEQUENCE |
| 1 | M. | DNA | Proprotein | ATGACTTCCTCTGCTAGTAAATTCTATACCATCTT |
| | truncatula | | encoding | CATTTTTGTCTGCCTTGCCTTTCTCTTTATTTCCA |
| | MtDef5FL | | | CATCTGGTACAAATTAATCAAATAACCTATGTTAA |
| | | | | TAATTTCTCATTTATTAATCCTTATTTATGTTCTT |
| | | | | AATTTGCTTCATTGTTATTTTCTTGCTTTCTATGT |
| | | | | GTAGAGGTGGAAGCAAAACTTTGTCAAAAGCGAAG |
| | | | | TACAACATGGTCAGGACCTTGTCTTAACACAGGAA |
| | | | | ACTGCAAAAGACAATGCATTAATGTGGAGCATGCT |
| | | | | ACTTTTGGTGCTTGTCATCGTCAAGGCTTTGGTTT |
| | | | | TGCTTGCTTCTGCTACAAAAAATGTGCTCCAAAGA |
| | | | | GTATATTGTTAACATTATTCTTTAAAACGATTATT |
| | | | | AAAATGCCAAAACTAAAATATAACAATATAAATGT |
| | | | | AACAATTAATCATTATGATCGATGCATATCACCTC |
| | | | | CAGATATATGTGGTACAAATAATCAAACAATCTAT |
| | | | | ATTAATAATTGTTCATTTTCTAATCCATATTTTTG |
| | | | | TGTTTTTAATTTTTCATATTTTTTTTTTTTTTTAT |
| | | | | ATGTAGAGGTGGAACCTAAACTTTGTGAAAGGCGA |
| | | | | AGCAAAACATGGTCAGGACCTTGTCTTATCTCAGG |
| | | | | AAATTGTAAAAGACAGTGCATCAATGTTGAGCATG |
| | | | | CAACTTCTGGTGCTTGTCACCGTCAAGGCATTGGT |
| | | | | TTTGCTTGCTTCTGCAAGAAAAAATGTTGA |
| 2 | M. | AA | Proprotein¹ | mtssaskfytififvclaflfistseveaKLCQKR |
| | truncatula | | | STTWSGPCLNIGNCKRQCINVEHATFGACHRQGFG |
| | MtDef5FL1 | | | FACFCYKKCapkkvepKLCERRSKTWSGPCLISGN |
| | | | | CKRQCINVEHATSGACHRQGIGFACFCKKKC |
| 3 | M. | AA | Signal | MTSSASKFYTIFIFVCLAFLFISTSEVEA |
| | truncatula | | peptide | |
| | MtDef5 SP | | | |
| 4 | M. | AA | Defensin | KLCQKRSTTWSGPCLNIGNCKRQCINVEHATFGAC |
| | truncatula | | peptide | HRQGFGFACFCYKKC |
| | MtDef5 | | | |
| | Defensin | | | |
| | Peptide A | | | |
| 5 | M. | AA | Defensin | KLCERRSKTWSGPCLISGNCKRQCINVEHATSGAC |
| | truncatula | | peptide | HRQGIGFACFCKKKC |
| | MtDef5 | | | |
| | Defensin | | | |
| | Peptide B | | | |
| 6 | M. | AA | Linker | APKKVEP |
| | truncatula | | peptide | |
| | MtDef5 | | | |
| | Linker | | | |
| | Peptide 1 | | | |
| 7 | M. | AA | Protein² | CQKRSTTWSGPCLNTGNCKRQCINVEHATFGACHR |
| | truncatula | | | QGFGFACFCYKKCapkkvepKLCERRSKTWSGPCL |
| | MtDef5FL1 | | | ISGNCKRQCINVEHATSGACHRQGIGFACFCKKKC |
| 8 | Medicago | AA | Defensin | KVCQKRSKTWSGPCLNIGNCKRQCVDVENATFGAC |
| | truncatula | | peptide | HRQGFGFACFCYKKC |
| 9 | Cicer | AA | Defensin | KLCQRRSKTWSGPCIITGNCKNQCKNVEHATFGAC |
| | arietinum* | | peptide | HRQGFGFACFCYFNCH |
| 10 | M. | AA | Defensin | NICKRKSTTWSGPCLNTGNCKNQCINVEHATFGAC |
| | truncatula | | peptide | HQDGFGFACFCYFNC |
| 11 | M. | AA | Defensin | NTCQRKSKTWSGPCLNTANCKNQCISKEPPATFGA |
| | truncatula | | peptide | CHRDGIGFACFCYFNC |
| 12 | Nicotiana | AA | Defensin | KVCQRRSKTWSGPCINTGNCSRQCKNQEDGRFGAC |
| | sylvestris | | peptide | HRSGIGFACFCYFNC |
| 13 | Solanum | AA | Defensin | KVCQRRSKTWSGPCINTGNCSRQCKQQEDARFGAC |
| | lycopersicum | | peptide | HRSGFGFACFCYFKC |
| 14 | Citrus | AA | Defensin | KVCQLRSKTWSGLCLNIGNCSRQCKQQEDARFGAC |
| | sinensis | | peptide | HRQGIGFACFCYFKC |
| 15 | C. | AA | Defensin | KVCQRRSKTWSGLCLNIGNCSRQCKQQEDARFGAC |
| | clementina | | peptide | HRQGIGFACFCYFKC |
| 16 | S. tuberosum | AA | Defensin | KVCQRRSQTWSGMCINTGNCSRQCKQQEDARFGAC |
| | | | peptide | HQNGIGFACFCYFTCK |
| 17 | C. sinensis | AA | Defensin | KVCQRRSKTWSGPCLNIGKCSRHCKQQEDARYGAC |
| | | | peptide | YRQGTGYACFCYFEC |
| 18 | C. | AA | Defensin | KVCQRRSKTWSGPCLNIGKCSRQCKQQEYARYGAC |
| | clementina | | peptide | YRQGAGYACYCYFNC |
| 19 | C. | AA | Defensin | KVCQRRSKTWSGPCLNIGKCSRQCKQQEYARYGAC |
| | clementina | | peptide | YRQGAGYACYCYFNC |
| 20 | Amborella | AA | Defensin | KLCQKRSRTWSGFCANSNNCSRQCKNLEGARFGAC |
| | trichopoda | | peptide | HRQRIGLACFCYFNC |
| 21 | Sesamum | AA | Defensin | KICQRMSKTWSGVCLNSGNCDRQCRNWERAQHGAC |
| | indicum* | | peptide | HRRGLGFACLCYFKC |
| 22 | A. thaliana | AA | Defensin | RICERRSKTWTGFCGNTRGCDSQCKRWERASHGAC |
| | 1.4 | | peptide | HAQFPGFACFCYFNC |
| 23 | A. halleri | AA | Defensin | RICERRSKTWTGFCGNTRGCDSQCKSWERASHGAC |
| | | | peptide | HAQFPGFACFCYFNC |
| 24 | A. lyrata | AA | Defensin | RICERRSKTWTGFCGNTRGCDSQCKSWERASHGAC |
| | | | peptide | HAQFPGFACFCYFNC |
| 25 | Capsella | AA | Defensin | RICQRRSKTWTGFCGNTRGCDSQCKRWERASHGAC |
| | rubella | | peptide | HAQFPGFACFCYFNC |
| 26 | Eutrema | AA | Defensin | RVCERRSKTWTGFCGNTRGCDSQCKRWERASHGAC |
| | salsugineum | | peptide | HAQFPGFACFCYFNC |
| 27 | Brassica | AA | Defensin | RVCQRRSKTWTGFCGNTRGCDSQCKRWERASHGAC |
| | rapa | | peptide | HAQFPGFACFCYFNC |
| 28 | B. napus | AA | Defensin | RVCQRRSKTWTGFCGNTRGCDSQCKRWERASHGAC |
| | | | peptide | HAQFPGFACFCYFNC |
| 29 | B. napus | AA | Defensin | RVCQRRSKTWTGFCGNTRGCDSQCKRWERASHGAC |
| | Def5 | | peptide | HAQFPGFACFCYFNC |
| 30 | Arabis | AA | Defensin | RICERRSKTWTGFCANTRGCDSQCKRWERASHGAC |
| | alpina | | peptide | HAQFPGVACFCYFNC |
| 31 | Camelina | AA | Defensin | RICERRSKTWTGFCGNTRGCDSQCRRWEHASHGAC |
| | sativa | | peptide | HAQFPGFACFCYFNC |
| 32 | Camelina | AA | Defensin | RICERRSKTWTGFCGNTRGCDSQCRSWEGASHGAC |
| | sativa | | peptide | HAQFPGFACFCYFNC |
| 33 | Erythranthe | AA | Defensin | RLCERRSKTWTGFCGSSNNCNNQCRNWERASHGAC |
| | guttata | | peptide | HAQFPGFACFCYFNC |
| 34 | Ricinus | AA | Defensin | KVCQRRSKTWSGFCGSTKNCDRQCKNWEGALHGAC |
| | communis | | peptide | HAQFPGVACFCYFKC |
| 35 | Vitis | AA | Defensin | KVCQRPSKTWSGFCGSSKNCDRQCKNWEGAKHGAC |
| | vinifera | | peptide | HAKFPGVACFCYFNC |
| 36 | Cucumis | AA | Defensin | KVCERRSKTWSGWCGNIKHCDRQCKNWEGATHGAC |
| | sativus | | peptide | HAQFPGRACFCYFNC |
| 37 | Pyrus | AA | Defensin | RICQRRSKTWSGFCANTGNCNRQCTNWEGALHGAC |
| | bretschneideri | | peptide | HAQFPGVACFCYFRC |
| 38 | Prunus | AA | Defensin | RICQRRSKTWSGFCGNIGNCNRQCRNWEGALRGAC |
| | persica | | peptide | HAQSPGFACFCYFRC |
| 39 | MtDef5FL1_V1 | AA | Protein | KLCQAASTTWSGPCLNIGNCKRQCINVEHATFGAC |
| | | | | AAQGFGFACFCYKKCapkkvepKLCEAASKTWSGP |
| | | | | CLISGNCKRQCINVEHATSGACAAQGIGFACFCKK |
| | | | | KC |
| 40 | Fragaria | AA | Defensin | RICQRRSKTWTGLCANTGNCHRQCRNWEGAQRGAC |
| | vesca | | peptide | HAQFPGFACFCYFNC |
| 41 | Jatropha | AA | Defensin | KLCQRRSKTWSGFCGDPGKCNRQCRNWEGASHGAC |
| | curcas | | peptide | HAQFPGFACFCYFKC |
| 42 | Amborella | AA | Defensin | KVCQKKSQTWSGECGNTNHCKTQCQKYEDARFGAC |
| | trichopoda | | peptide | HSQGFGHACFCYFTC |
| 43 | Theobroma | AA | Defensin | KLCQKRSKTWTGPCIKTKNCDHQCRKWEKAQHGAC |
| | cacao | | peptide | HWQWPGFACFCYVNC |
| 44 | M. | AA | Defensin | KLCKRYSTEFHAICTDTGICKNACIYLEHAAFGAC |
| | Truncatula | | peptide | HRDGLGFACFCYFNC |
| 45 | Citrus | AA | Defensin | KQCSKRAQKWTGPCIKTGSCRNHCRKREGAVDGAC |
| | clementina | | peptide | HYDFPGFACFCYYNC |
| 46 | Citrus | AA | Defensin | KQCSKRAQKWTGPCIKTGSCRNHCRKREGAVDGAC |
| | sinensis | | peptide | HYDFPGFACFCYYNC |
| 47 | S. | AA | Defensin | IGCEKMSVTWSGPCFDTGGCNNQCINWEHAIHGAC |
| | lycopersicum | | peptide | HWDWTGPACYCYFC |
| 48 | Glycine max | AA | Defensin | GLCAKRSKTWSGWCGSSNNCDKQCRTKEGATHGAC |
| | | | peptide | HGNILKRACDCYFKC |
| 49 | Capsella | AA | Defensin | AGVCQRYSGSWTGICMSTSNCNTQCIERESAKYGA |
| | rubella | | peptide | CHFDGNGSACFCYFDC |
| 50 | B. napus | AA | Defensin | EVCQRYSGSWSGICLSSSNCNTQCIEREHAKYGAC |
| | | | peptide | HSDDNGLACFCYFDC |
| 51 | B. rapa | AA | Defensin | EVCQRYSGSWSGICLSSSNCNTQCIEREHAKYGAC |
| | | | peptide | HSDDNGLACFCYFDC |
| 52 | B. napus | AA | Defensin | EVCQRYSGSWSGICLSSSNCNTQCIEREHAKYGAC |
| | | | peptide | HSDDNGLACFCYFDC |
| 53 | B. oleracea | AA | Defensin | EVCQRYSGSWSGICLSSSNCNTQCIEREHAKYGAC |
| | | | peptide | HSDDNGLACFCYFDC |
| 54 | A. lyrata | AA | Defensin | GVCQRYSGSWEGVCIFSSNCNTQCIERESAKYGAC |
| | | | peptide | HRDDNGLACFCYFDC |
| 55 | Eutrema | AA | Defensin | EVCQRYSGSWKGFCFSTSNCNNQCIERENAKYGAC |
| | salsugineum | | peptide | HADSNGVACFCYFDC |
| 56 | Leersia | AA | Proprotein | meakaattvlvllllilggeaGKMCHDPSQTFKGM |
| | perrieri | | | CFHTMNCISSCTNEGYTGGYCTYLKHKCICTKPCv |
| | (A0A0D9VC | | | gegcedKICQQHSGTFKGICFNNNNCVSYCVAEQF |
| | R3)³ | | | TSGFCSGVVDRKCICTKECddkpptelslpppkkk |
| | | | | wpprrqhgqwqqpqrrcdrteerlcvvwgwllf |
| 57 | Leersia | AA | Protein | GKMCHDPSQTFKGMCFHTMNCISSCTNEGYTGGYC |
| | perrieri | | with two | TYLKHKCICTKPCvgegcedKICQQHSGTFKGICF |
| | | | defensin | NNNNCVSYCVAEQFTSGFCSGVVDRKCICTKEC |
| | | | peptide | |
| | | | sequences | |
| | | | separated | |
| | | | by a linker | |
| | | | peptide | |
| 58 | Leersia | AA | Signal | meakaattvlvllllilggea |
| | perrieri | | peptide | |
| 59 | Leersia | AA | Defensin | GKMCHDPSQTFKGMCFHTMNCISSCTNEGYTGGYC |
| | perrieri | | Peptide A | TYLKHKCICTKPC |
| 60 | Leersia | AA | Linker | vgegced |
| | perrieri | | peptide | |
| | | | Domain | |
| 61 | Leersia | AA | Defensin | KICQQHSGTFKGICFNNNNCVSYCVAEQFTSGFCS |
| | perrieri | | Peptide B | GVVDRKCICTKEC |
| 62 | ZmESR-6 | AA | Defensin | KLCSTTMDLLICGGAIPGAVNQACDDTCRNKGYTG |
| | | | peptide | GGFCNMKIQRCVCRKPC |
| 63 | MtDefFL1 | DNA | protein | AAACTTTGTCAAAAGCGAAGTACAACATGGTCAGG |
| | encoding | | encoding | ACCTTGTCTTAACACAGGAAACTGCAAAAGACAAT |
| | sequence | | | GCATTAATGTGGAGCATGCTACTTTTGGTGCTTGT |
| | | | | CATCGTCAAGGCTTTGGTTTTGCTTGCTTCTGCTA |
| | | | | CAAAAAATGTGCTCCAAAGAAGGTGGAACCTAAAC |
| | | | | TTTGTGAAAGGCGAAGCAAAACATGGTCAGGACCT |
| | | | | TGTCTTATCTCAGGAAATTGTAAAAGACAGTGCAT |
| | | | | CAATGTTGAGCATGCAACTTCTGGTGCTTGTCACC |
| | | | | GTCAAGGCATTGGTTTTGCTTGCTTCTGCAAGAAA |
| | | | | AAATGTTGA |
| 64 | codon- | DNA | | AAGTTGTGCCAAAAAAGATCTACTACCTGGAGTGG |
| | optimized | | | TCCTTGCCTTAATACCGGAAATTGTAAAAGACAGT |
| | mature | | | GCATCAATGTTGAGCATGCTACTTTTGGTGCTTGT |
| | defensin | | | CATAGACAAGGTTTTGGTTTCGCTTGTTTCTGTTA |
| | sequence of | | | CAAGAAATGTGCTCCAAAGAAAGTTGAACCTAAGT |
| | MtDef5FL1 | | | TGTGTGAGAGAAGATCTAAAACTTGGTCTGGTCCA |
| | for Pichia | | | TGTTTGATTTCTGGTAACTGTAAGAGACAATGTAT |
| | expression | | | CAACGTTGAACACGCTACAAGTGGAGCCTGCCATA |
| | | | | GACAAGGTATTGGTTTCGCCTGCTTTTGCAAGAAG |
| | | | | AAGTGTTAATGA |
| 65 | sequence | DNA | Vector | TCGAGAAAAGA |
| | encoding the | | sequence | |
| | XhoI | | | |
| | restriction | | | |
| | enzyme site | | | |
| | and the | | | |
| | KEX2 | | | |
| | protease site | | | |
| 66 | Two stop | DNA | Vector | TAATGAGAATTC |
| | codons, | | sequence | |
| | along with an | | | |
| | EcoRI | | | |
| | restriction | | | |
| | enzyme site | | | |
| 67 | M. | DNA | | ATGACTTCCTCTGCTAGTAAATTCTATACCATCTT |
| | truncatula | | | CATTTTTGTCTGCCTTGCCTTTCTCTTTATTTCCA |
| | MtDef5FL2 | | | CATCTGAGGTGGAAGCAAAACTTTGTCAAAAGCGA |
| | | | | AGTACAACATGGTCAGGACCTTGTCTTAACACAGG |
| | | | | AAACTGCAAAAGACAATGCATTAATGTGGAGCATG |
| | | | | CTACTTTTGGTGCTTGTCATCGTCAAGGCTTTGGT |
| | | | | TTTGCTTGCTTCTGCTACAAAAAATGTGCTCCAAA |
| | | | | GAGTATATTGTTAACATTATTCTTTAAAACGATTA |
| | | | | TTAAAATGCCAAAACTAAAATATAACAATATAAAT |
| | | | | GTAACAATTAATCATTATGATCGATGCATATCACC |
| | | | | TCCAGATATATGTGAGGTGGAACCTAAACTTTGTG |
| | | | | AAAGGCGAAGCAAAACATGGTCAGGACCTTGTCTT |
| | | | | ATCTCAGGAAATTGTAAAAGACAGTGCATCAATGT |
| | | | | TGAGCATGCAACTTCTGGTGCTTGTCACCGTCAAG |
| | | | | GCATTGGTTTTGCTTGCTTCTGCAAGAAAAAATGT |
| | | | | TGA |
| 68 | M. | AA | Proprotein¹ | mtssaskfytififvclaflfistseveaKLCQKR |
| | truncatula | | | STTWSGPCLNTGNCKRQCINVEHATFGACHRQGFG |
| | MtDef5FL2 | | | FACFCYKKCapksilltlffktiikmpklkynnin |
| | | | | vtinhydrcisppdicevepKLCERRSKTWSGPCL |
| | | | | ISGNCKRQCINVEHATSGACHRQGIGFACFCKKKC |
| 69 | M. | AA | Protein | KLCQKRSTTWSGPCLNTGNCKRQCINVEHATFGAC |
| | truncatula | | with two | HRQGFGFACFCYKKCapksilltlffktiikmpkl |
| | MtDef5FL2 | | defensin | kynninvtinhydrcisppdicevepKLCERRSKT |
| | | | peptide | WSGPCLISGNCKRQCINVEHATSGACHRQGIGFAC |
| | | | sequences | FCKKKC |
| | | | separated | |
| | | | by a linker | |
| | | | peptide | |
| 70 | M. | AA | Linker | apksilltlffktiikmpklkynninvtinhydrc |
| | truncatula | | peptide | isppdicevep |
| | MtDef5 | | | |
| | Linker | | | |
| | Peptide 2 | | | |
| 71 | M. | DNA | Proprotein | ATGACTTCCTCTGCTAGTAAATTCTATACCATCTT |
| | truncatula | | encoding | CATTTTTGTCTGCCTTGCCTTTCTCTTTATTTCCA |
| | MtDef5FL3 | | | CATCTGAGGTGGAAGCAAAACTTTGTCAAAAGCGA |
| | | | | AGTACAACATGGTCAGGACCTTGTCTTAACACAGG |
| | | | | AAACTGCAAAAGACAATGCATTAATGTGGAGCATG |
| | | | | CTACTTTTGGTGCTTGTCATCGTCAAGGCTTTGGT |
| | | | | TTTGCTTGCTTCTGCTACAAAAAATGTGCTCCAAA |
| | | | | GAGTATATTGTTAACATTATTCTTTAAAACGATTA |
| | | | | TTAAAATGCCAAAACTAAAATATAACAATATAAAT |
| | | | | GTAACAATTAATCATTATGATCGATGCATATCACC |
| | | | | TCCAGATATATGTGGTACAAATAATCAAACAATCT |
| | | | | ATATTAATAATTGTTCATTTTCTAATCCATATTTT |
| | | | | TGTGTTTTTAATTTTTCATATTTTTTTTTTTTTTT |
| | | | | ATATGTAGAGGTGGAACCTAAACTTTGTGAAAGGC |
| | | | | GAAGCAAAACATGGTCAGGACCTTGTCTTATCTCA |
| | | | | GGAAATTGTAAAAGACAGTGCATCAATGTTGAGCA |
| | | | | TGCAACTTCTGGTGCTTGTCACCGTCAAGGCATTG |
| | | | | GTTTTGCTTGCTTCTGCAAGAAAAAATGTTGA |
| 72 | M. | AA | Proprotein¹ | mtssaskfytififvclaflfistseveaKLCQKR |
| | truncatula | | | STTWSGPCLNTGNCKRQCINVEHATFGACHRQGFG |
| | MtDef5FL3 | | | FACFCYKKCapksilltlffktiikmpklkynnin |
| | | | | vtinhydrcisppdicgtnnqtiyinncsfsnpyf |
| | | | | cvfnfsyfffflyvevepKLCERRSKTWSGPCLIS |
| | | | | GNCKRQCINVEHATSGACHRQGIGFACFCKKKC |
| 73 | M. | AA | Protein | KLCQKRSTTWSGPCLNTGNCKRQCINVEHATFGAC |
| | truncatula | | with two | HRQGFGFACFCYKKCapksilltlffktiikmpkl |
| | MtDef5FL3 | | defensin | kynninvtinhydrcisppdicgtnnqtiyinncs |
| | | | peptide | fsnpyfcvfnfsyfffflyvevepKLCERRSKTWS |
| | | | sequences | GPCLISGNCKRQCINVEHATSGACHRQGIGFACFC |
| | | | separated | KKKC |
| | | | by a linker | |
| | | | peptide | |
| 74 | M. | AA | Linker | apksilltlffktiikmpklkynninvtinhydrc |
| | truncatula | | Peptide | isppdicgtnnqtiyinncsfsnpyfcvfnfsyff |
| | MtDef5 | | | fflyvevep |
| | Linker | | | |
| | Peptide 3 | | | |
| 75 | Medicago | AA | proprotein | marsvplvstifvfllllvatgpsmvaeaRTCESQ |
| | truncatula | | | SHKFKGPCASDHNCASVCQTERFSGGHCRGFRRRC |
| | (MtDef4; | | | FCTTHC |
| | SEQ ID | | | |
| | NO: 111 in | | | |
| | U.S. Pat. | | | |
| | No. 7,825,297) | | | |
| 76 | Medicago | AA | Defensin | RTCESQSHKFKGPCASDHNCASVCQTERFSGGHCR |
| | truncatula | | peptide | GFRRRCFCTTHC |
| | (MtDef4; | | | |
| | SEQ ID | | | |
| | NO: 112 in | | | |
| | U.S. Pat. | | | |
| | No. 7,825,297) | | | |
| 77 | Medicago | AA | Defensin | RTCESQSHKFKGPCASDHNCASVCQTERFSGGHCR |
| | truncatula | | Peptide | GFRRRCFCTTHC |
| | (MtDef4; | | consensus | |
| | SEQ ID | | | |
| | NO: 71 in | | | |
| | U.S. Pat. No. | | | |
| | 7,825,297) | | | |
| 78 | Medicago | AA | proprotein | mekkslaglcflflvlfvaqeivvteaRTCENLAD |
| | sativa | | | KYRGPCFSGCDTHCTTKENAVSGRCRDDFRCWCTK |
| | (MsDef1) | | | RC |
| 79 | Medicago | AA | Defensin | RTCENLADKYRGPCFSGCDTHCTTKENAVSGRCRD |
| | sativa | | peptide | DFRCWCTKRC |
| | (MsDef1) | | | |
| 80 | Nicotiana | AA | proprotein | marslcfmafailammlfvayevqaRECKTESNIF |
| | alata (NaD1) | | | PGICITKPPCRKACISEKFTDGHCSKILRRCLCTK |
| | | | | PCVFDEKMTKTGAEILAEEAKTLAAALLEEEIMDN |
| 81 | Nicotiana | AA | Defensin | RECKTESNTFPGICITKPPCRKACISEKFTDGHCS |
| | alata (NaD1) | | peptide | KILRRCLCTKPC |
| 82 | Tomato- | AA | proprotein | marsiffmaflvlammlfvtyevea QQICK |
| | Solanum | | | APSQTFPGLCFMDSSCRKYC |
| | lycopersicum | | | IKEKFTGGHCSKLQRKCLCT KPC |
| | (TPP3) | | | VFDKISSEVKATLGEEAKTLSEVVLEEEIMME |
| 83 | Tomato- | AA | Defensin | QQICKAPSQTFPGLCFMDSSCRKYC |
| | Solanum | | peptide | IKEKFTGGHCSKLQRKCLCTKPC |
| | lycopersicum | | | |
| | (TPP3) | | | |
| 84 | Hexima | AA | Protease | EEKKN |
| | Linker | | sensitive | |
| | | | linker | |
| 85 | Hexima | AA | Protease | X.sub.1X.sub.2X.sub.3X.sub.4X.sub.5 |
| | Linker | | sensitive | where X.sub.1 is E (glu) or D |
| | | | peptide | (asp), X.sub.2 is E (glu) or D |
| | | | | (asp), X.sub.3 is K (lys) or R |
| | | | | (arg), X.sub.4 is K (lys) or R |
| | | | | (arg) and X.sub.5 is N (asn) or Q |
| | | | | (gln) |
| 86 | 1^{st} internal | AA | Protease | DSKPNPTKEEEPAKKPDEVSVKSGGPEVSED |
| | propeptide | | sensitive | |
| | from IbAMP | | peptide | |
| | polyprotein | | | |
| | precursor | | | |
| | from the | | | |
| | seeds of I. | | | |
| | balsamina | | | |
| 87 | 2^{nd} internal | AA | Protease | ANAEEAAAAIPEASEELAQEEAPVYSED |
| | propeptide | | sensitive | |
| | IbAMP | | peptide | |
| | polyprotein | | | |
| 88 | 3^{rd} internal | AA | Protease | QNAEEAAAAIPEATEKAQEAPVYSED |
| | propeptide | | sensitive | |
| | IbAMP | | peptide | |
| | polyprotein | | | |
| 89 | 4^{th} internal | AA | Protease | QNAEEAAAAVAIPEASEKAQEGPVYSED |
| | propeptide | | sensitive | |
| | IbAMP | | peptide | |
| | polyprotein | | | |
| 90 | 5^{th} internal | AA | Protease | SNAADEVATPEDVEPG |
| | propeptide | | sensitive | |
| | IbAMP | | peptide | |
| | polyprotein | | | |
| 91 | 6^{th} internal | AA | Protease | HNAAEEATLKAFEEEAAREQPVYSED |
| | propeptide | | sensitive | |
| | IbAMP | | peptide | |
| | polyprotein | | | |
| 92 | 7^{th} internal | AA | Protease | QSAEEAAAFGQGEVTASLMLIMFKACPCMGPVPSV |
| | propeptide | | sensitive | |
| | IbAMP | | peptide | |
| | polyprotein | | | |
| 93 | Synthetic | AA | Synthetic | GGGGS |
| | Gly₄Ser | | spacer | |
| | sequence | | peptide | |
| 94 | Synthetic | AA | Synthetic | SGGGGS |
| | S(Gly₄Ser)n | | spacer | |
| | sequence | | peptide | |
| 95 | Arabidopsis | AA | proprotein¹ | meripslaslvslliifatvvnqtraSICNDRLGL |
| | At5g38330 | | | CDGCDQRCKAKHGPSCESKCDGPVGMLLCTCTYEC |
| | | | | gptKLCNGGLGNCGESCNEQCCDRNCAQRYNGGHG |
| | | | | YCNTLDDFSLCLCKYPC |
| 96 | Arabidopsis | AA | Defensin | SICNDRLGLCDGCDQRCKAKHGPSCESKCDGPVGM |
| | At5g38330 | | peptide I | LLCTCTYEC |
| 97 | Arabidopsis | AA | Defensin | KLCNGGLGNCGESCNEQCCDRNCAQRYNGGHGYCN |
| | At5g38330 | | peptide II | TLDDFSLCLCKYPC |
| 98 | Arabidopsis | AA | proprotein¹ | metvtslvfivnlliiftsvvnqargDICIDGLGY |
| | At4g30070 | | | CNNCDERCKAKHGPSSESSCDRSVGVPLCKCYYEC |
| | | | | esppsppappKKCDGGAGICSQRCQGQCCDMNCAQ |
| | | | | KYIGGHGFCNTLGTFSFCQCEYPC |
| 99 | Arabidopsis | AA | Defensin | DTCIDGLGYCNNCDERCKAKHGPSSESSCDRSVGV |
| | At4g30070 | | peptide I | PLCKCYYEC |
| 100 | Arabidopsis | AA | Linker | ESPPSPPAPP |
| | At4g30070 | | Peptide | |
| | | | peptide | |
| 101 | Arabidopsis | AA | Defensin | KKCDGGAGICSQRCQGQCCDMNCAQKYIGG HGFC |
| | At4g30070 | | peptide II | NTLGTFSFCQCEYPC |
| 102 | Nicotiana | AA | Protease | EEKKN |
| | alata | | sensitive | |
| | proteinase | | peptide | |
| | inhibitor | | motif | |
| 103 | Synthetic | AA | spacer | KESGSVSSEQLAQFRSLD |
| | spacer | | peptide | |
| | peptide | | | |
| 104 | Synthetic | AA | spacer | EGKSSGSGSESKST |
| | spacer | | peptide | |
| | peptide | | | |
| 105 | Synthetic | AA | spacer | GSAGSAAGSGEF |
| | spacer | | peptide | |
| | peptide | | | |
| 106 | MtDef4 | AA | Defensin | RGFRRR |
| | gamma-core | | fragment | |
| | peptide loop | | | |
| 107 | Yeast | AA | Protease | KREAEA |
| | protease | | cleavage | |
| | cleavage site | | site | |
| | sequence | | | |
| 108 | RsAFP2 | AA | Defensin | QKLCQRPSGTWSGVCGNNNACKNQCIRLEKAR |
| | | | peptide | HGSCNYVFPAHKCICYFPC |
| 109 | DmAMP1 | AA | Defensin | ELCEKASKTWSGNCGNTGHCDNQCKSWEGAAH |
| | | | peptide | GACHVRNGKHMCFCYFNC |
| 110 | Psd1 | AA | Defensin | KTCEHLADTYRGVCFTNASCDDHCKNKAHLIS |
| | | | peptide | GTCHNWKCFCTQNC |
| 111 | Medicago | AA | Proprotein¹ | mtssankfytifvfvclalllistseveaKVCQKR |
| | sativa | | | SKTWSGPCLNTGNCKRQCINVEHATFGACHRQGFG |
| | MsDef5 | | | FACFCYRKCapkkvepKLCERRSKTWSGPCLNSGH |
| | ortholog | | | CKRQCINVEHATSGACHRQGLGIACFCKKKC |
| 112 | Lotus | AA | Proprotein¹ | mankisnsslflvffilvamveltmgGGRCSELVS |
| | japonicum | | | RCGSSFPDCDQICKSQHSSINGKGFCNDNICTCYY |
| | | | | DCgpplppgpvvRKCLAEIGPCSGQCNSNCAGRFK |
| | | | | GATGTCNAAFGNLCICQYTC |
| 113 | synthetic | ssDNA | primer | CCGTACGTTGGCTGATTACA |
| 114 | synthetic | ssDNA | primer | CCGTACGTTGGCTGATTACA |
| 115 | synthetic | ssDNA | primer | CCTAAACTTTGTGAAAGGCGAAG |
| 116 | synthetic | ssDNA | primer | GGTGACAAGCACCAGAAGT |
| 117 | Medicago | AA | Defensin | KVCQKRSKTWSGPCLNTGNCKRQCINVEHATF |
| | sativa | | Peptide A | GACHRQGFGFACFCYRKC |
| | MsDef5 | | | |
| | ortholog | | | |
| 118 | Medicago | AA | Defensin | KLCERRSKTWSGPCLNSGHCKRQCINVEHATSGAC |
| | sativa | | Peptide B | HRQGLGIACFCKKKC |
| | MsDef5 | | | |
| | ortholog | | | |
| 119 | Lotus | AA | Defensin | GGRCSELVSRCGSSFPDCDQTCKSQHSSTN |
| | japonicus | | Peptide A | GKGFCNDNICTCYYDC |
| 120 | Lotus | AA | Defensin | RKCLAEIGPCSGQCNSNCAGRFKGATGTC |
| | japonicus | | Peptide B | NAAFGNLCICQYTC |

¹In proproteins, apoplast targeting peptides (signal peptide) are bold and underlined, defensin peptides are in upper case, peptides separating the defensin peptides are underlined, and any vacuolar targeting peptide (where present) is double underlined.
²Protein as expressed in Pichia.
³Umprot protein database on the World Wide Web at uniport.org.

### Example 6

### Additional Analysis of Antifungal Defensins

Materials And Methods

Fungal Cultures and Growth Medium

Fusarium graminearum PH-1 was routinely cultured on complete medium (CM). For production of conidia, the fungus was inoculated into carboxymethyl cellulose medium (CMC) and cultured for 2-5 days at 30° C. with shaking at 180 rpm. Neurospora crassa was routinely cultured on Vogel's agar media. Botrytis cinerea, Fusarium thapsinum, F. verticilloides, F. oxysporum, and F. virguliforme were cultured onto PDA (Potato dextrose agar). In vitro antifungal assays were performed using synthetic fungal medium (SFM) without calcium as described previously (Broekaert et al.,1990).

Expression and Purification of MtDef5 and its Variants

The codon-optimized DNA sequences encoding the mature defensin sequence of MtDef5FL1 and its variants were each cloned between the Xhol and Xbal sites of pPICZαA vector in frame with the a-factor secretion signal sequence without the Glu-Ala repeats at the Kex2 signal cleavage site. The resulting recombinant pPICZαA-dimeric defensin was then linearized by digestion with Sad restriction enzyme and transformed into competent cells of P. pastoris X33 by electroporation. Transformants were initially selected on YPD (10 g/L yeast extract, 20 g/L peptone, 20 g/L glucose and 20 g/L agar) plates containing 150 μg/mL of zeocin, followed by restreaking the transformants on YPD plates containing 500 μg/mL of zeocin. Finally, transformants that survived at higher zeocin concentration were used for production of each dimeric defensin. The MtDef5FL1 expressing P. pastoris X33 was grown at 28° C. for 2 days on YPD agar. Then, a single colony was inoculated into 50 mL of YPD broth and grown overnight at 30° C. on a rotary shaker at 225 rpm. Pichia culture was then inoculated into 500 mL of buffered minimal glycerol media (BMGY/L—10 g yeast extract, 20 g peptone, 100 ml 1M potassium phosphate of pH 6.0, 100 mL 10× yeast nitrogen base, 2 mL 500× biotin and 100 mL 10× glycerol) and grown overnight at 30° C. on a rotary shaker at 225 rpm. After overnight growth, cells were pelleted by centrifugation at 8,000 rpm for 10 minutes at room temperature (RT) and re-suspended in 1,000 mL of buffered methanol complex medium (BMM/L—100 mM potassium phosphate of pH 6.0, 100 mL 10× yeast nitrogen base, 2 mL 500× biotin and 5 mL methanol). The culture was grown for 5 days at 25° C., and 5 mL of methanol was added to the culture every 24 hours to maintain induction of gene expression. After induction, cells were pelleted by centrifugation at 4,000 rpm for 5 minutes, and the supernatant was retained. The pH of the supernatant was adjusted to 6.0 and CM-Sephadex C-25 cation-exchange resin (Amersham Biosciences, Piscataway, N.J.) was added to bind the protein and the culture was incubated in an incubator at 4° C. and 110 rpm for 1 to 2 days. The slurry was poured through Miracloth® into a Buchner funnel and the resin was collected and packed into a FPLC (Fast protein liquid chromatography) column. The resin was extensively washed with a binding buffer (25 mM sodium acetate, pH 6.0), and the bound protein was then eluted in 1 M NaCl, 50 mM Tris, pH 7.6. FPLC fractions were collected and concentrated at 4° C. in an Amicon Stirred Cell concentrator (Cole-Parmer, Vernon Hills, Ill.) with a membrane that had a molecular weight cut-off (MWCO) of 3,000. The concentrated fraction was dialyzed in 1,000 MWCO tubing against 50 mM Tris, pH 7.6. The dialysate was filter-sterilized through a 0.2 μm filter and lyophilized. The lyophilized protein was re-suspended in nuclease-free water and the protein concentration was determined by NanoDrop spectrophotometer (Thermo Scientific). Purity and size of the protein isolate were assessed using a 15% SDS-PAGE gel.

Antifungal Assays

The antifungal activity of MtDef5 was measured in an in vitro assay using 96-well microtiter plates. Fifty microliters (μL) of each protein dilution were added to each well of the microtiter plate, which contained 50 μL of spore suspension (105 spores mL-1) of F. graminearum and N. crassa prepared in 2× SFM and Vogel's medium, respectively. To determine the broad-spectrum antifungal activity of MtDef5FL1 against F. thapsinum, F. verticilloides, F. oxysporum, F. virguliforme, and B. cinerea-spore suspension(s) were prepared in 2× SFM at a concentration of 105 spores mL-1. A control group without MtDef5FL1 was used as a negative control. To monitor the visible phenotypic effects of MtDef5FL1 on conidial germination and growth of fungal hyphae (at 14 to 16 hours after treatment with MtDef5FL1), bright-field images were taken using the transmitted light channel in a Leica SP8-X confocal microscope. The quantitative fungal growth inhibition by MtDef5FL1 was estimated by measuring the absorbance at 595 nm using a Tecan Infinite M200 Pro (Tecan Systems Inc., San Jose, Calif.) microplate reader at different time points.

SYTOX Green (SG) Uptake Assay

SG uptake assays were conducted as described (Sagaram et al., 2011). N. crassa and F. graminearum conidia (504, of 5×104/mL) were allowed to germinate overnight at room temperature in Vogel's liquid and 2X SFM media, respectively, in a 10 mm microwell of 35 mm glass bottom microwell dish with a No. 1.5 cover glass. Wet filter papers were placed in the container to prevent drying of the conidial suspension. After 16 hours, MtDef5FL1 (50 μL, at concentrations of 90 nM and 180 nM) and 1 μL of 0.5 μM SG (Thermo Fisher Scientific, N.Y.) were added to both fungal hyphae. Samples were gently agitated for 2 hours and mounted on a microscope for imaging. A Leica SP8-X confocal microscope was used for all confocal imaging. Control plates with SG, and without MtDef5FL1, were used as a negative control.

Protein-Lipid Interactions Using Lipid Blot Assays

To test the binding properties of MtDef5 and its γ-core motif variants, a protein-lipid overlay experiment was performed using PIP Strips™ (2×6 cm nitrocellulose membranes) that are spotted with 100 pmol of various biologically active lipids, respectively (Echelon Biosciences, Salt Lake City, Utah). To determine relative degree of the binding of MtDef5, PIP Array (Echelon Biosciences, UT) pre-spotted with concentration gradient of various phosphoinositides was used. Briefly, lipid strips and arrays were blocked with 10 mL of blocking buffer, PBS-T/3% fat free BSA for 12-16 h at 4° C. with gentle agitation. The blocking buffer was discarded and the lipid strips and arrays were incubated with 5 mL of PBS-T/3% fat free BSA, containing 3 μg/ml of MtDef5 or its variants for 60 min at 4° C. After hybridization, protein solution was discarded and the strips and arrays were then washed with PBS-T three times with gentle agitation for 20 minutes each at room temperature. The protein-lipid interactions were detected by subsequently incubating lipid strips and arrays with rabbit anti-MtDef5 derived synthetic peptide (1 μg/ml) antibody diluted in 5 mL of blocking buffer with gentle agitation for 60 min at 4° C. The lipid strips and arrays were washed with PBS-T two times with gentle agitation, for 15 minutes each wash, at room temperature. HRP-conjugated Goat Anti-Rabbit IgG secondary antibody (Thermo Scientific, Cat no: SA 1-9510) diluted 1:4000 in blocking buffer was added to the lipid strips and arrays and incubated for 60 min at 4° C. After two washes with PBS-T, chemiluminescence was detected using SuperSignal® West Femto Maximum Sensitivity substrate kit (Thermo Scientific, Cat no: 34094) following the manufacturer's protocol.

Oligomerization of MtDef5FL1 and its Variants

To determine the oligomeric status of MtDef5FL1 and its γ-core motif variants, protein cross-linking experiments were performed in the presence or absence of PIPs cross-linked with bis[sulfosuccinimidyl] suberate (BS3) substrate. Briefly, MtDef5 and its γ-core motif variants, prepared in 1× PBS buffer, at 1.5 mg/ml (5 μl) were incubated with 2.73 mM PI(3)P or PI(4)P or PI(5)P (1.5 μl) at room temperature for 30 min. The cross-linking reaction was initiated by addition of freshly dissolved water soluble 12.5 mM BS3 (0.5 μl) and incubated for 30 min at room temperature. After crosslinking, samples were reduced with 100 mM dithiothreitol (DTT) and separated on a 4-20% (Bio-Rad, TGX Stain-Free™ precast gels) SDS-PAGE. The oligomerization pattern was visualized using a Bio-Rad ChemiDoc XRS+ system.

Transmission Electron Microscopy (TEM)

TEM imaging was performed according to the procedure described by Adda et al.,(2009). Briefly, MtDef5FL1 cross-linked protein complex reaction mixture (10 μl) was applied to 400-mesh copper grids coated with a thin layer of carbon for 2 min. Excess material was removed by blotting, and samples were negatively stained twice with 10 μl of a 2% (wt/vol) uranyl acetate solution (Electron Microscopy Services, Hatfield, Pa.). The grids were air-dried and viewed using a transmission electron microscope operated at 80 kV. Digital images were acquired using a LEO 912 AB energy filter TEM operated at 120 kV. In some cases large fields of view were acquired by montaging.

Confocal Microscopy/Live Cell Imaging

MtDef5FL1 defensin was labeled with DyLight550 using labeling kit following the protocol provided by the manufacturer (Thermo Scientific, USA). Laser-scanning confocal microscopy was used to monitor internalization of fluorescently labeled MtDef5 into N. crassa and F. graminearum cells. For MtDef5FL1 internalization assays, N. crassa conidia (50 μL of 105/mL) and germlings were placed in 10 mm microwell of 35 mm glass bottom microwell dishes (MatTek Corporation, Ashland Mass.). Wet filter papers were placed in the petri dishes to prevent drying of the conidial suspension. The bottom of the 10 mm microwell has a No. 1.5 cover glass and hence facilitates direct observation of the sample without mechanical disruption due to sample transfer. Fifty microliter of 2 μM Dylight550-MtDef5FL1 was added to the conidia or the germlings of N. crassa and the plates were placed immediately on the microscope for imaging. A Leica SP8-X confocal microscope was used for all confocal imaging. DyLight-MtDef5FL1 was excited at 550 nm and fluorescence detected at 560-600 nm, and FM4-64 dye was excited at 550 nm and fluorescence detected at 690-800 nm. Imaging was carried out at room temperature and images were analyzed using ImageJ and Bitplane Imaris softwares. qRT-PCR analysis

Total RNA was extracted with the E.Z.N.A Plant RNA Kit (Omega, USA) according to the manufacturer's instructions. The total RNA was treated with TURBO DNA-free™ Kit (Ambion, USA) and quantified using a NanoDrop Spectrophotometer (Thermo Scientific, USA)., according to the manufacturer's instructions. Following RNA extraction, cDNA was synthesized from 1 μg total RNA using iScript™ cDNA Synthesis Kit (Bio-Rad, USA). Real-time qPCR analysis was performed using SsoAdvanced™ Universal IT SYBR Green Supermix (Bio-Rad, USA) on a Bio-Rad CFX384 Touch™ Real-Time PCR Detection, according to the manufacturer instructions. For each sample, a reaction without RT was performed as a control for contamination by genomic DNA. Each experiment was performed in triplicates with at least three independent samples. Primer pair sequences used for amplifying ubiquitin 10 were forward, CCGTACGTTGGCTGATTACA (SEQ ID NO:113); reverse, CGTCTTTCCCGTTAGGGTTT (SEQ ID NO:114) and for MtDef5 forward, CCTAAACTTTGTGAAAGGCGAAG (SEQ ID NO:115); reverse, GGTGACAAGCACCAGAAGT (SEQ ID NO:116). Expression was normalized to the ubiquitin 10 gene as an endogenous control (Kaur et al., 2012). The resulting data were analyzed using relative quantification based on the AACt(delta-delta-Ct) method.

### Western Blot Analysis

Total protein extracts were prepared by grinding leaf tissue of wild-type Col-0, Null line and transgenic Arabidopsis lines in 200 μl of HEPES protein extraction buffer, according to the protocol described by the Kim et al., 2008 with slight modifications (100 mm N-2-hydroxyethylpiperazine-N′-2-ethanesulfonic acid (HEPES), pH 7.5, 5 mm ethylenediaminetetraacetic acid (EDTA), 10 mm dithiothreitol (DTT), 1 mm phenylmethylsulfonyl fluoride (PMSF), 5% glycerol). The extract was centrifuged at 14,000 g for 40 min, and the supernatant obtained was used to determine the protein concentration using Bradford assay kit (Bio-Rad, USA). Equal amounts (45 μg) of protein samples were separated on 4-20% (Bio-Rad, TGX™ precast gels) SDS-PAGE gels and electroblotted onto a low fluorescence polyvinylidine difluoride (PVDF) membrane (Azure Biosystems, USA). Membranes were blocked in 10 mL of fluorescent blot blocking buffer (Azure Biosystems, USA) for 1 h at room temperature with gentle agitation. The membranes were incubated for 1 h with the rabbit anti-MtDef5 derived primary antibody (1 μg/ml), followed by incubation for 1 h with the AzureSpectra α-rabbit 550 fluorophore-conjugated secondary antibody (Azure Biosystems, USA). Images were taken using the background quenching sheets using RGB with an Azure Biosystems c600. ImageJ was used to quantify the protein accumulation in three transgenic lines. Purified MtDef5 protein (100 ng) was used as a positive control.

Hyaloperonospora arabidopsidis inoculations and spore count measurements

Spores of an oomycete oligate biotrophic pathogen H. arabidopsidis Noco2 isolate were harvested from infected WTArabidopsisplants and the concentration was adjusted to 5×104 spores/mL. Spore solution was sprayed onto 4-week-old transgenic, WT and nullArabidopsisplants, which were subsequently incubated in a growth chamber maintained at 17° C. for 7 days with a 10-h day : 14-h night cycle. The aerial parts of two plants from each line were cut and placed in a 50-mL tube containing 1 mL of sterile water. The tube was vortexed (about 15 s) and the spores were counted using a haemocytometer. For each transgenic, WT and null line, two biological replications were used. Values are normalized to Col-0. Error bars represent standard deviation between two independent biological replicates.

### Results

MtDef5FL1 exhibits extremely potent broad-spectrum antifungal activity.

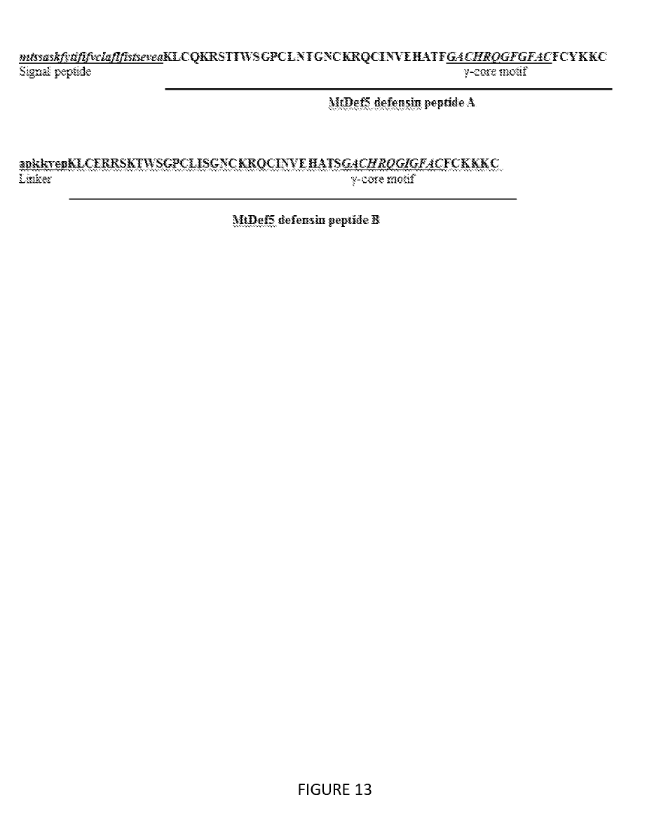

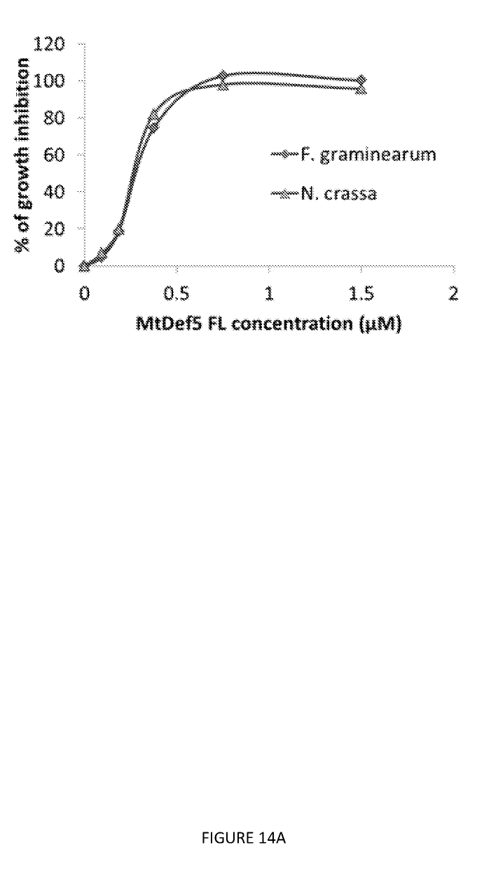

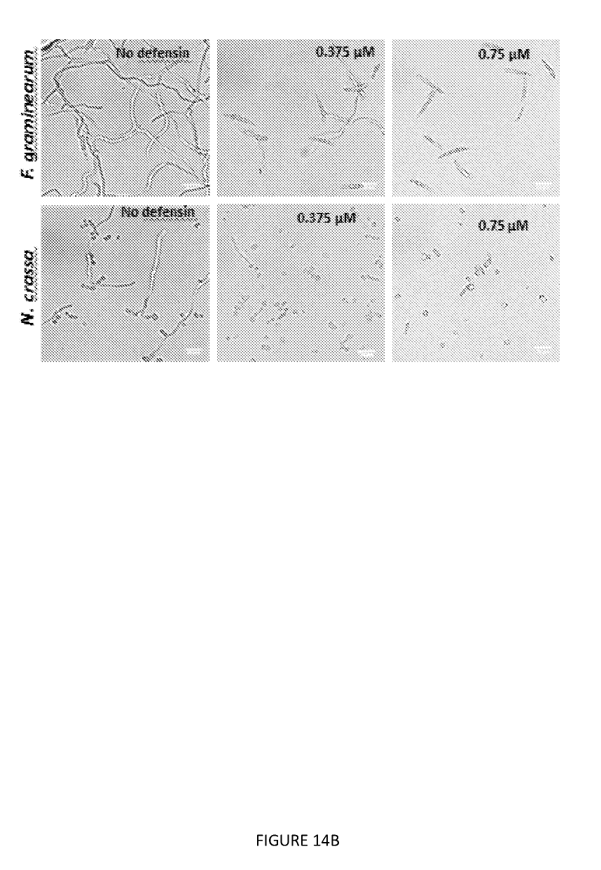

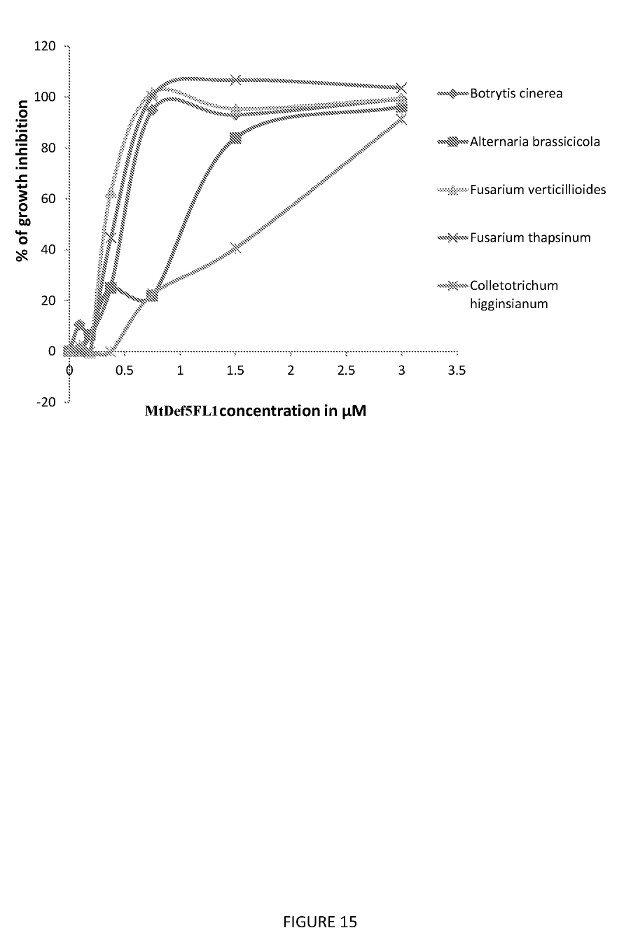

MtDef5FL1 (FIG. 13) was expressed in P. pastoris and the protein secreted into the medium was purified using our previously published protocols. The antifungal activity of MtDef5FL1 was tested against F. graminearum and N. crassa. MtDef5 inhibited the growth of F. graminearum and Neurospora crassa with an IC50 value of 0.25-0.3 μM and minimal inhibitory concentration (MIC or IC100) of 0.7-0.75 μM (FIG. 14). This compares with the IC50 value of 0.75-1.5 μM and MIC value of 6 μM for MtDef4 against the F. graminearum. Based on the MIC values of these two defensins against this fungus, MtDef5FL1 is 3 to 5-fold more potent than MtDef4. MtDef5FL1 also exhibits potent antifungal activity against several other filamentous fungal pathogens including F. virguliforme, Alternaria brassicicola, Colletotrichum higginsianum and Botrytis cinerea (Bci) (FIG. 15). Thus, MtDef5FL1 is a potent antifungal protein with strong potential as an antifungal agent in transgenic crops.

MtDef5FL1 Permeabilizes Fungal Plasma Membrane

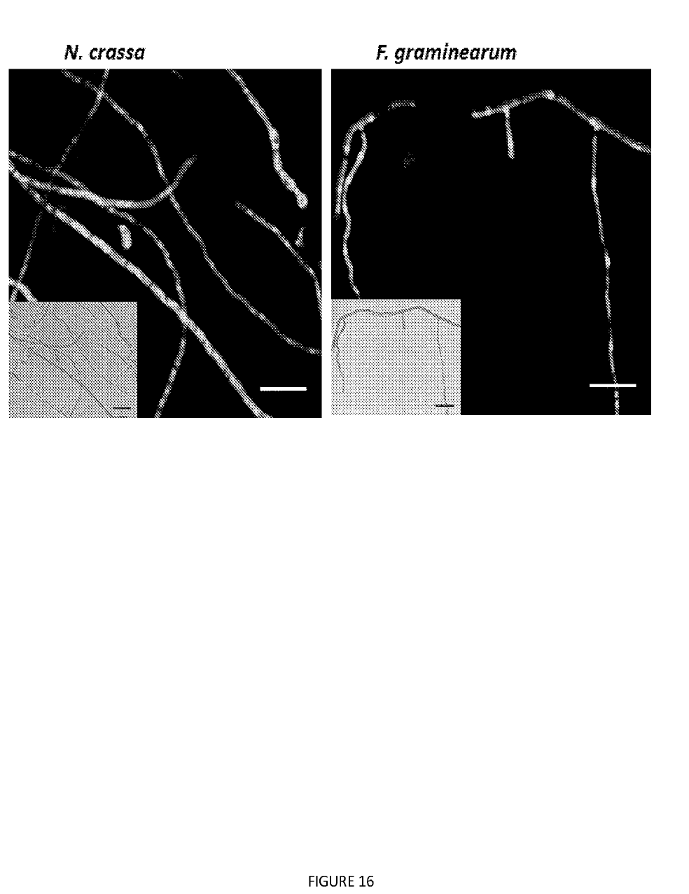

Many plant defensins act on fungal cells by permeabilizing their plasma membrane. The SYTOX™ Green (SG) uptake assay was used to test if MtDef5FL1 permeabilizes the plasma membrane of F. graminearum, and N. crassa. SG is a dye which is only taken up by cells with a compromised plasma membrane and its fluorescence increases >500-fold upon binding to nucleic acids thus allowing quantitative analysis and fluorescence microscopy. The uptake of SG was assessed using confocal microscopy in fungal hyphae treated with 0.75 μM MtDef5FL1. SG uptake was visible in the hyphal cells of both fungi after 3 hr of treatment with MtDef5FL1 (FIG. 16). This result indicates that membrane permeabilization is one of the key early events in the antifungal action of the MtDef5FL1.

MtDef5FL1 is internalized by fungal cells and diffusely localized within the cytoplasm

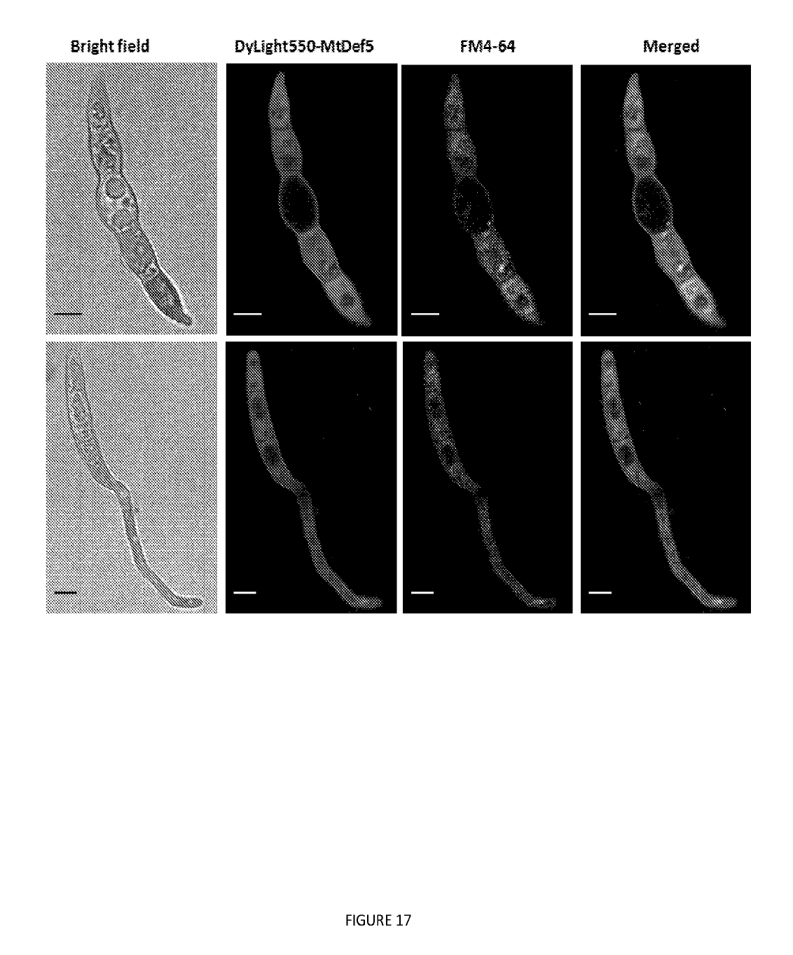

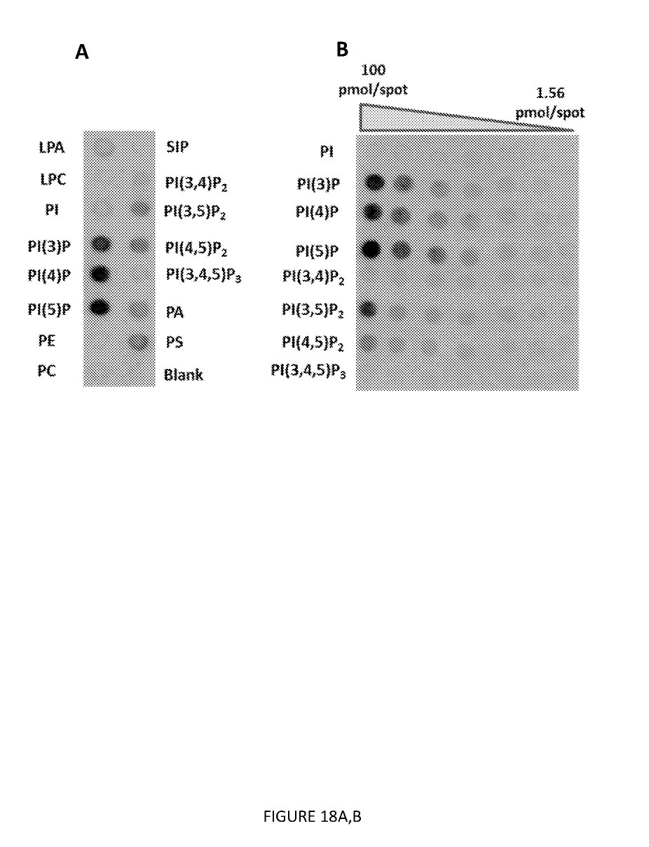

To determine MtDef5FL1 internalization and its subcellular targets in fungal cell, we labeled the protein with the fluorophore DyLight550 using the labeling kit following the protocol provided by the manufacturer (Thermo Scientific, USA). F. graminearum conidia and germlings (4 hr) were treated with DyLight550-MtDef5FL1 at the final concentration of 3μM and the membrane selective dye FM4-64 (Hickey et al., 2002) for 3 hr. As shown in FIG. 17, MtDef5FL1 is internalized by fungal cells treated with fluorescently labeled defensin. Upon entry to the cells, DyLight550-MtDef5FL1 does not localize within any specific organelle but readily diffuses into the cytoplasm of conidia and germlings of F. graminearum.

MtDef5FL1 binds strongly to phosphatidylinositol monophosphates (PIPs)

To investigate the spectrum of potential lipid ligands interacting with MtDef5FL1, protein-lipid overlay assay was performed with various biologically active lipids immobilized on a PIP strip. Using this assay, we have determined that MtDef5FL1 strongly binds to phosphatidylinositol 3-phosphate (PI3P), phosphatidylinositol 4-phosphate (PI4P) and phosphatidylinositol 5-phosphate (PISP). Although the MtDef5FL1 showed much higher affinity for phosphatidylinositol monophosphates, it also binds to a lesser extent to other functionally important membrane lipids such as phosphatidylinositol 3,5-bisphosphate (PI3,5P2) phosphatidylinositol 4,5-bisphosphate (PI4,5P2), phoshatidic acid (PA) and phosphatidylserine (PS) (FIG. 18A). Consistent with the initial results obtained using a PIP strip, we observed binding of MtDef5FL1 to all three PIPs even at 12.5 pmoles per spot indicating high affinity of this defensin to these bioactive phospholipids (FIG. 18B).

MtDef5FL1 forms oligomers in presence of PIPs leading to formation of nanonet-like structures

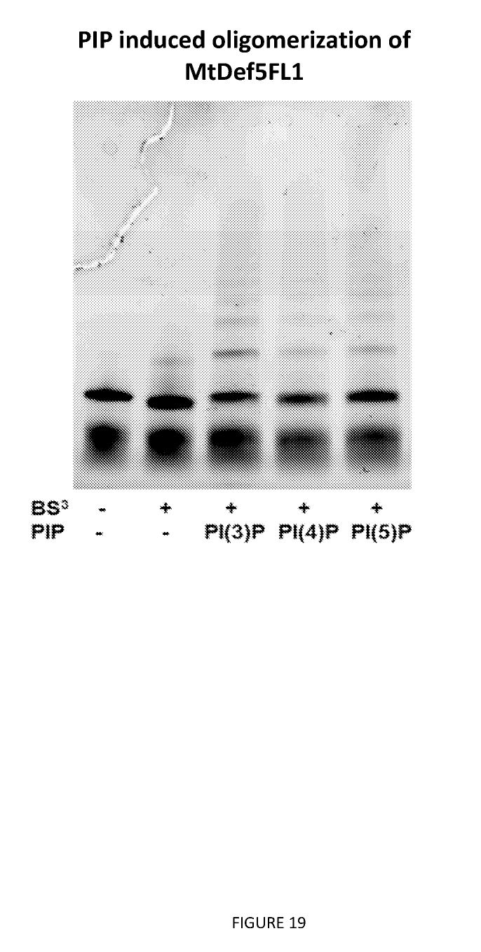

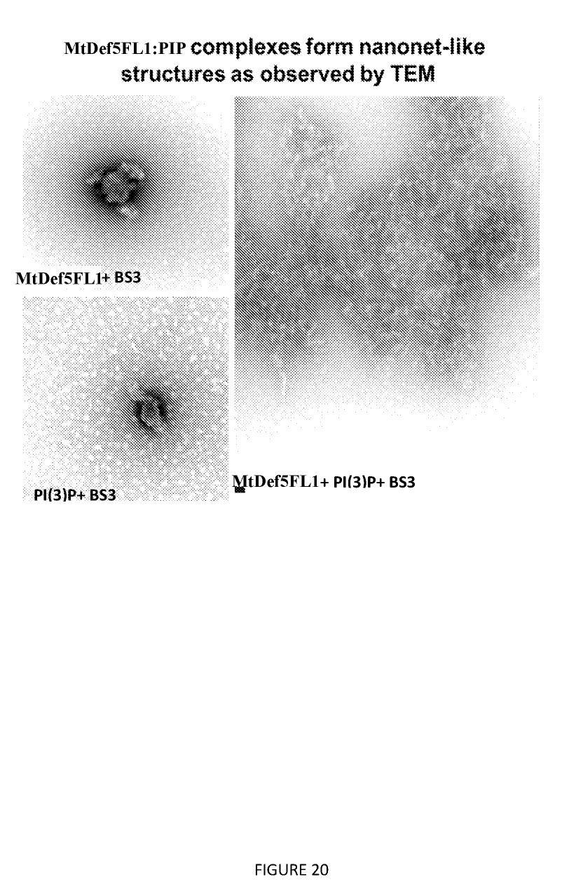

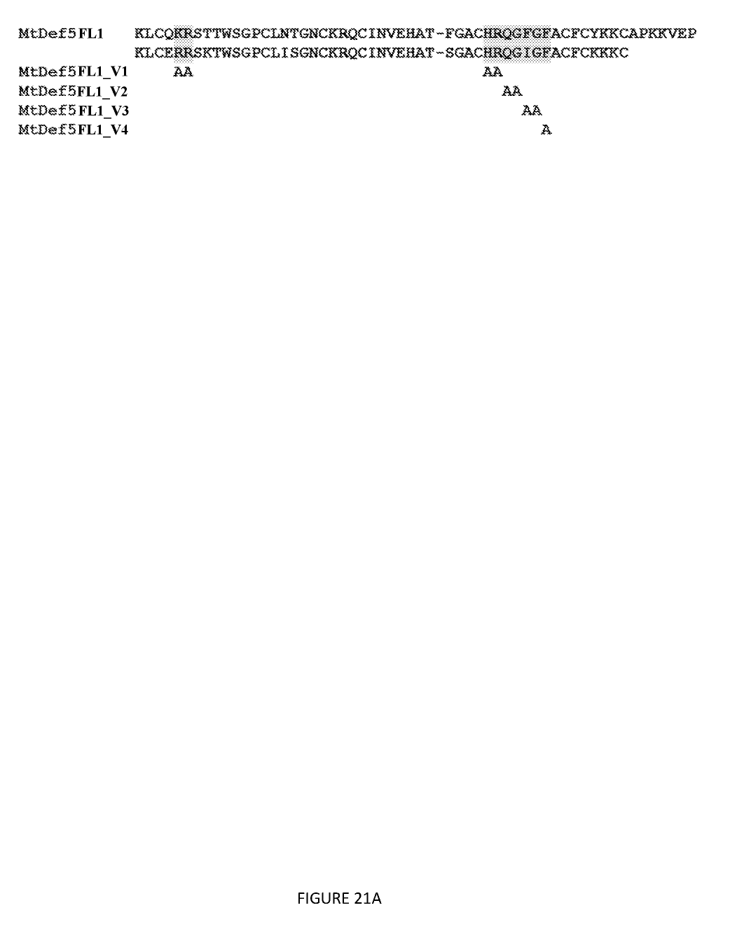

We tested in vitro ability of MtDef5FL1 to oligomerize by conducting protein cross-linking experiments. MtDef5FL1 formed higher-order oligomers only in the presence of PIPs. Separation of cross-linked protein complexes on gradient SDS-PAGE indicates the formation of trimers, tetramers and high-molecular-weight oligomers (FIG. 19). The MtDef5FL1-PIP oligomers were visualized under the transmission electron microscope. Nanonet-like structures were observed only in the presence of PIP. In contrast, PIP-B53 or MtDef5FL1-B53 substrate alone did not form nanonet-like structures (FIG. 20).

MtDef5FL1 γ-core motif variants except MtDef5FL1_V4 exhibit reduced antifungal activity; however, all variants exhibit reduced membrane permeabilization and retain wild-type phospholipid binding

We generated four MtDef5FL1 variants to identify the amino acid residues involved in the antifungal activity, membrane permeabilization, PIP binding, and oligomerization of MtDef5FL1. MtDef5FL1 variants were expressed in P. pastoris, purified and tested for their antifungal activity against F. graminearum.

MtDef5FL1 γ-core motif variants exhibit different antifungal activity and ability to permeabilize fungal plasma membrane

As shown in FIG. 8A, MtDef5FL1_V3 and MtDef5FL1_V4 retain wild-type antifungal activity. However, MtDef5FL1_V1 (SEQ ID NO:39) and MtDef5FL1_V2 show 4-fold and 2-fold reduction in their antifungal activity against F. graminearum relative to that of the wild-type MtDef5FL1. These variants were also tested for their ability to permeabilize the plasma membrane of F. graminearum using the SG uptake assay. As shown in FIG. 8B, MtDef5FL1_V3 exhibited faster uptake of SG than the wild-type MtDef5FL1 during the first 30 min. MtDef5FL1_V2 and MtDef5FL1_V4 permeabilized the plsma membrane of the fungus in a manner similar to that of the wild-type MtDef5FL1. In contrast, MtDef5FL1_V1 lost its ability to permeabilize the fungal plasma membrane almost completely.

MtDef5FL1 γ-core motif variants retain wild-type PIPs binding

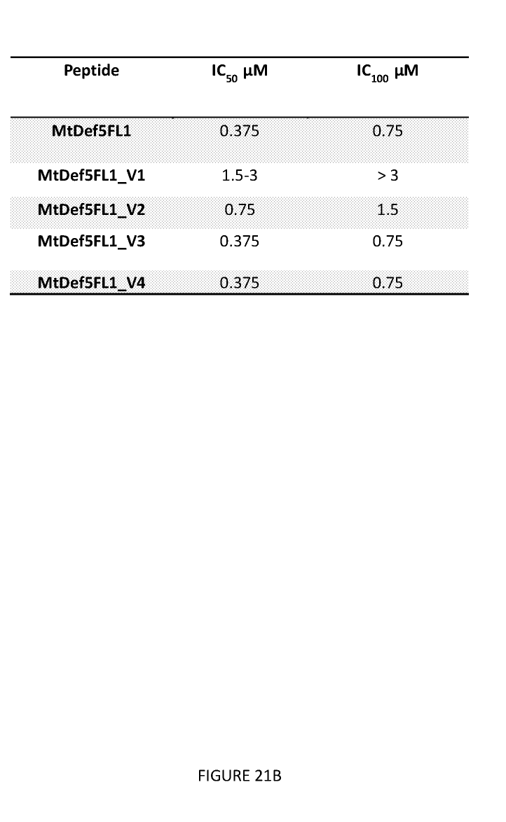

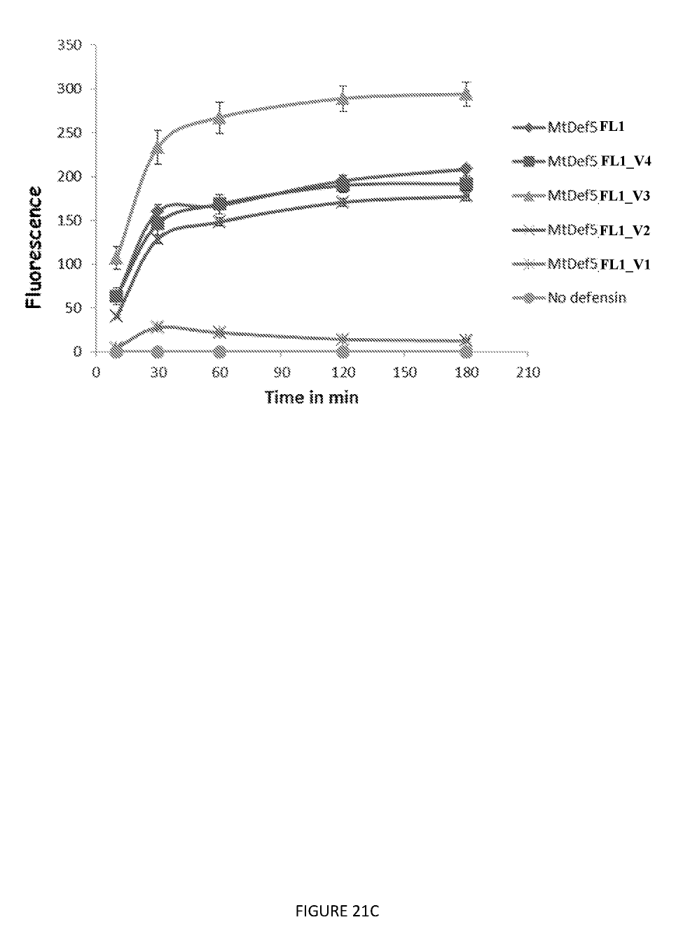

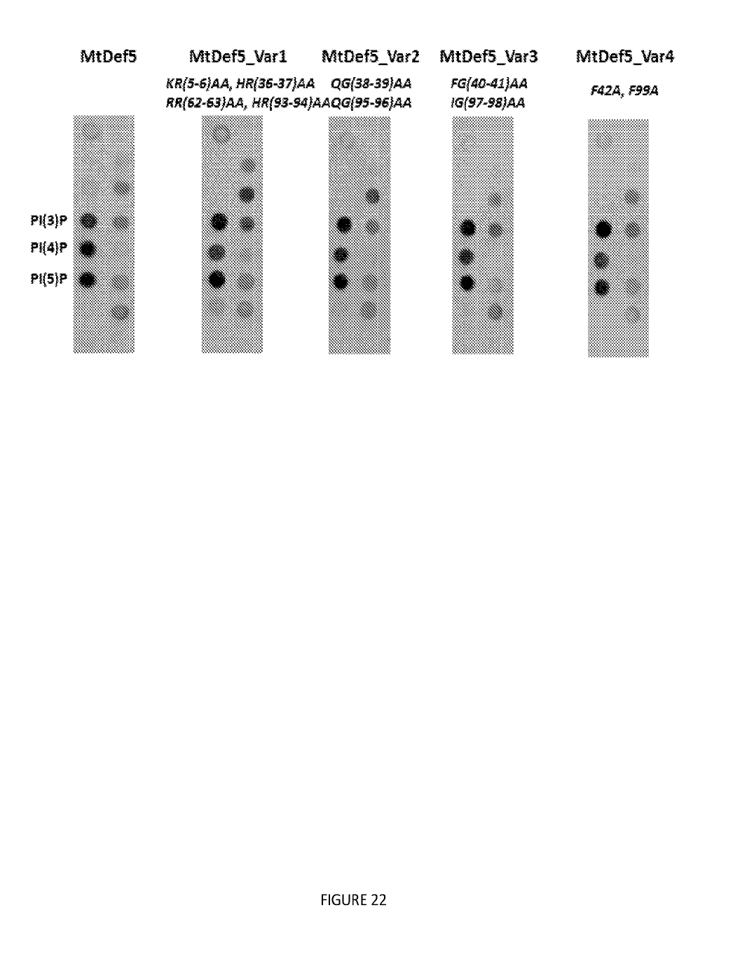

To determine the critical residues involved in PIP binding, amino acid residues of the MtDef5FL1 γ-core motif were mutated to alanine. A total of four variants of MtDef5FL1 were generated to determine the effect of these mutations on PIP binding (FIG. 21). We have determined that MtDef5FL1 γ-core motif variants were not altered in their lipid binding profiles and retained wild-type MtDef5FL1 PIPs binding affinity and specificity (FIG. 22).

MtDef5FL1 γ-core motif variants exhibit much reduced oligomerization in presence of PIPs

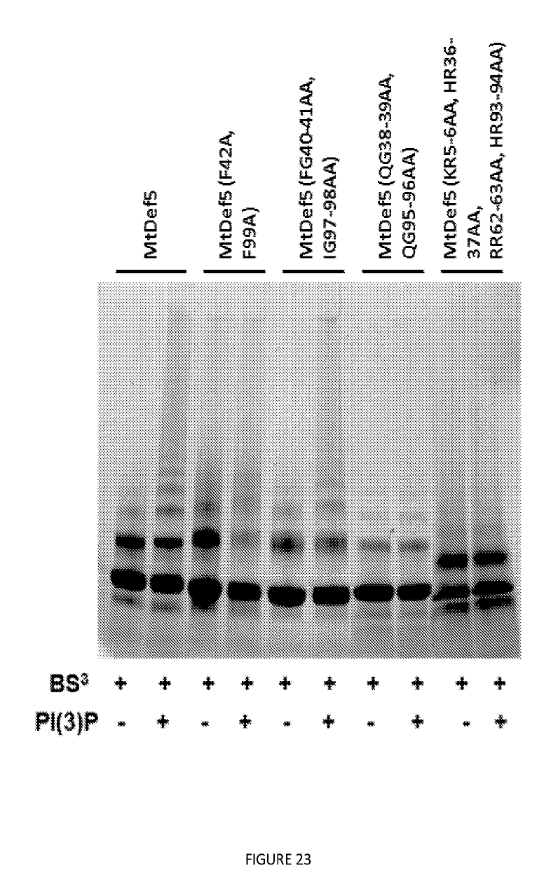

MtDef5FL1 γ-core motif variants (MtDef5FL1_V2, MtDef5FL1_V4) exhibited much reduced oligomerization in the presence of PIPs with the exception of the MtDef5FL1_V3 which showed a slightly reduced oligomerization relative to that of the wild-type MtDef5FL1. In contrast, MtDef5FL1_V1 showed no formation of higher order oligomers in presence of PIP. As expected, MtDef5FL1-BS3 substrate alone did not form higher order oligomers (FIG. 23).

Expression Analysis of MtDef5FL1 in transgenic Arabidopsis thaliana lines

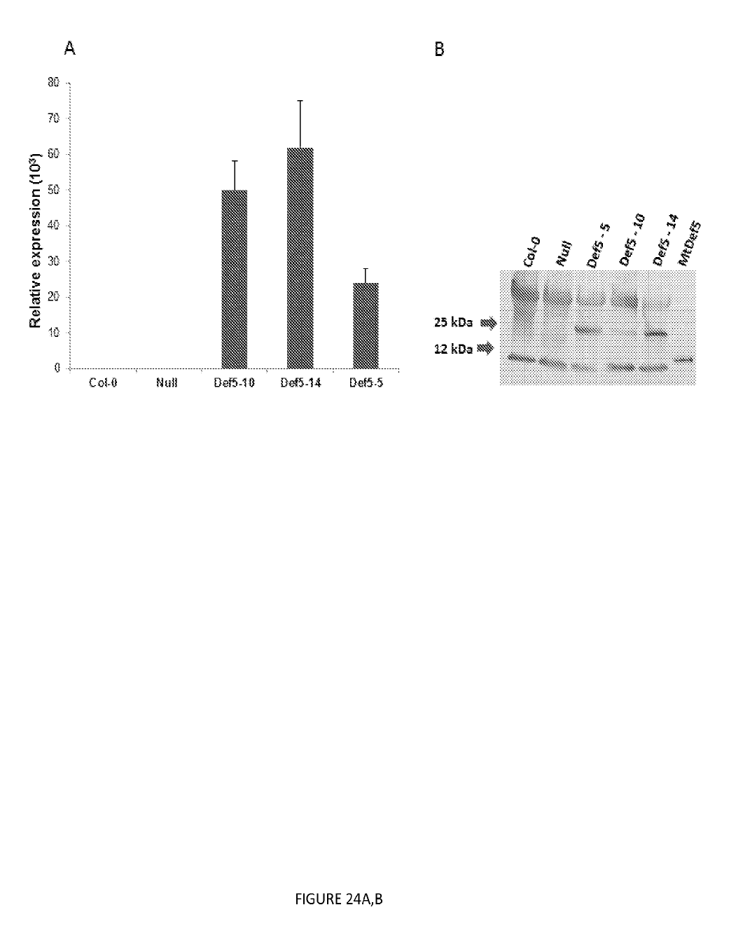

RT-qPCR was carried out to determine the transcript level of the MtDef5FL1 gene in 4-week old transgenic Arabidopsis thaliana lines, wild type Col-0 and null lines. The qRT-PCR experiments have revealed high expression of the MtDef5FL1 transcript in three homozygous lines. The highest level of MtDef5FL1 expression was observed in line Def5-14. Transgenic lines Def5-5, Def5-10 and Def5-14 showed 24000-, 50000-, 63000-fold expression of MtDef5FL1, respectively, compared to wild type and null lines. No MtDef5FL1 gene expression was observed in wild type and null lines (FIG. 24A).

To determine MtDef5FL1 protein expression in transgenic lines, the immunoblot analysis was performed using a primary antibody raised against MtDef5FL1 synthetic peptide. Western blot analysis of the total leaf protein isolated from these lines has shown expression of ˜24 kD protein and not the expected 11.9 kD protein. This ˜24 kD protein is not present in the wild-type and null lines. Protein levels measured by immunoblot analysis were different in transgenic lines. Lines Def5-5 and Def5-14 have high levels of protein expression equal to that of MtDef5FL1 positive control (100 ng). However, the expression level of MtDef5FL1 protein in line Def5-10 is below 100 ng of MtDef5FL1 positive control (FIG. 24B).

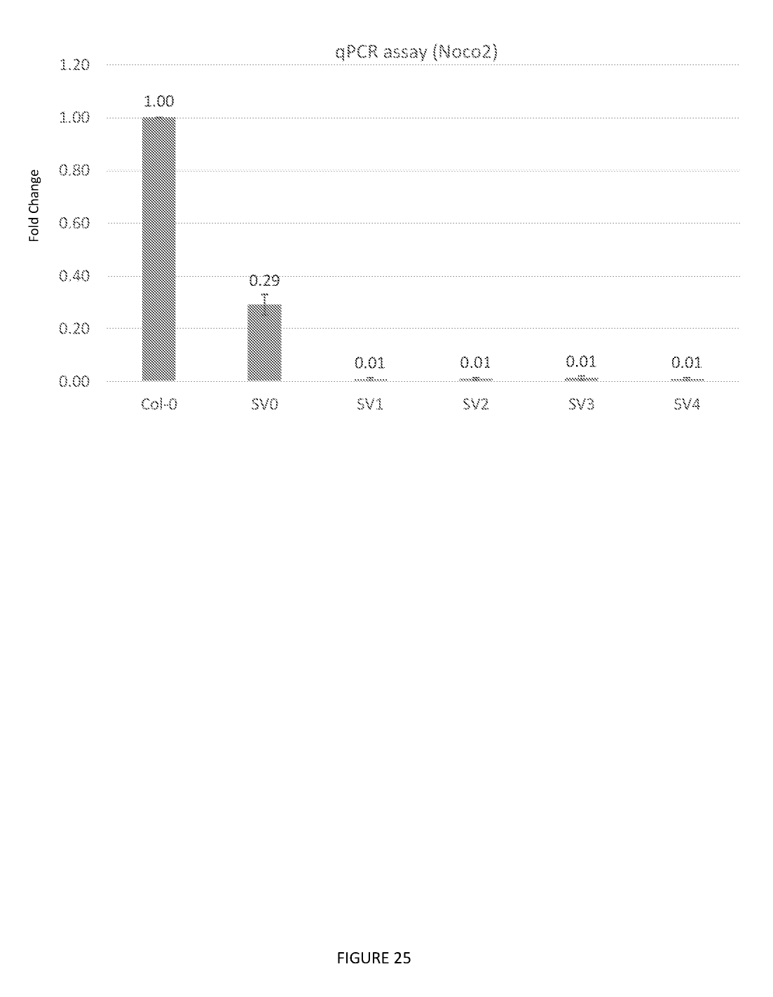

Transgenic Arabidopsis lines exhibit strong resistance to H. arabidopsidis. Transgenic Arabidopsis lines Def5-5, Def5-10 and Def5-14 lines were challenged with the spores of H. arabidopsidis eNoco5. As shown in FIG. 25, all transgenic lines exhibited strong resistance to this downy mildew disease as measured by the spore count, whereas the two Col-0 wild-type lines were susceptible to this pathogen. Null segregant lines derived from the transgenic line Def5-10 also exhibited strong resistance to this pathogen in this experiment. It is hypothesized that the H. arabidopsidis resistance observed in null segregant lines obtained from transgenic line MtDef5-10 could be due to an epigenetic effect. It is predicted that resistance to H. arabidopsidis Noco5 will remain stable in subsequent generations of the MtDef5-10 transgenic lines, but will be lost in subsequent generations of the non-transgenic, null segregant lines derived from the transgenic line Def5-10.

### References for Example 6

Adda C G, Murphy V J, Sunde M, Waddington L J, Schloegel J, Talbo G H, Vingas K, Kienzle V, Masciantonio R, Howlett G J, Hodder A N, Foley M, Anders R F (2009) Plasmodium falciparum merozoite surface protein 2 is unstructured and forms amyloid-like fibrils. Mol Biochem Parasitol 166:159-171. doi: 10.1016/j.molbiopara.2009.03.012

Broekaert, W. F., Terras, F. R. G., Cammue, B. P. A. & Vanderleyden, J. An automated quantitative assay for fungal growth inhibition. FEMS Microbiology Letters 69 (1-2), 55-59, doi:10.1111/j.1574-6968.1990.tb04174.x (1990).

Kaur J, Thokala M, Robert-Seilaniantz A, Zhao P, Peyret H, Berg H, Pandey S, Jones J, Shah D (2012) Subcellular targeting of an evolutionarily conserved plant defensin MtDef4.2 determines the outcome of plant-pathogen interaction in transgenic Arabidopsis. Mol Plant Pathol 13:1032-1046. doi: 10.1111/j.1364-3703.2012.00813.x

Kim C Y, Bove J, Assmann S M (2008) Overexpression of wound-responsive RNA-binding proteins induces leaf senescence and hypersensitive-like cell death. New Phytol 180:57-70. doi: 10.1111/j.1469-8137.2008.02557.x

Sagaram U S, Pandurangi R, Kaur J, Smith T J, Shah D M (2011) Structure-activity determinants in antifungal plant defensins msdefl and mtdef4 with different modes of action against Fusarium graminearum. PLoS One. doi: 10.1371/journal.pone.0018550

The breadth and scope of the present disclosure should not be limited by any of the above-described examples.

### Embodiments

The following numbered embodiments form part of the disclosure:

1. A recombinant nucleic acid molecule comprising a polynucleotide encoding a multimeric defensin polypeptide that comprises two defensin peptides separated by a spacer peptide that is resistant to cleavage by a plant endoproteinase, wherein the polynucleotide is operably associated with at least one heterologous polynucleotide that promotes transcription, transcript abundance, translation, or any combination thereof.

2. A recombinant nucleic acid molecule comprising a polynucleotide encoding a multimeric defensin polypeptide that comprises two defensin peptides separated by a heterologous spacer peptide that is resistant to cleavage by a plant endoproteinase.

3. The nucleic acid of embodiment 2, wherein the polynucleotide is operably associated with at least one heterologous polynucleotide that promotes transcription, transcript abundance, translation, or a combination thereof.

4. A recombinant polynucleotide encoding a polypeptide comprising a multimeric defensin polypeptide that has at least 70%, 75%, 80%, 85%, 90%, 95%, 98%, 99%, or 100% sequence identity across the entire length of a polypeptide selected from the group consisting of SEQ ID NO:57, SEQ ID NO:69, SEQ ID NO:73, and SEQ ID NO:112 or comprising a multimeric defensin polypeptide that has at least 92, 95%, 98%, 99%, or 100% sequence identity across the entire length of a polypeptide of SEQ ID NO:111, wherein the polynucleotide is operably associated with at least one heterologous polynucleotide that promotes transcription, transcript abundance, translation, or a combination thereof.

5. The nucleic acid molecule of any one of embodiments 1-4, wherein the multimeric defensin polypeptide inhibits fungal growth.

6. The nucleic acid molecule of any one of embodiments 1-4, wherein the multimeric defensin polypeptide wherein the multimeric defensin polypeptide, when expressed by or administered to a plant, can confer resistance to infection by a plant pathogenic fungus.

7. The nucleic acid molecule of any one of embodiments 1, 3, 4, 5, or 6, wherein the at least one heterologous polynucleotide comprises a promoter, a 5′ untranslated region (5′ UTR), a 3′ untranslated region (3′ UTR), an intron, a polyadenylation site, or any combination thereof.

8. The nucleic acid molecule of any one of embodiments 1 to 7, further comprising a polynucleotide encoding a localization peptide that is operably associated with the multimeric defensin.

9. The recombinant nucleic acid of embodiment 8, wherein the at least one localization peptide is an apoplast localization peptide, an endoplasmic reticulum localization peptide, a mitochondrial localization peptide, a plastid localization peptide, a vacuole localization peptide, or any combination thereof.

10. The recombinant nucleic acid of embodiment 8, wherein the localization peptide is a heterologous localization peptide that can direct an operably associated polypeptide to an extracellular or sub-cellular location that is different than the extracellular or sub-cellular location of a naturally occurring polypeptide comprising one or both of the defensin peptides.

11. The recombinant nucleic acid of any one of embodiments 1 to 10, wherein the spacer peptide does not contain the peptide sequence of SEQ ID NO:6, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, or SEQ ID NO: 100.

12. The nucleic acid molecule of any one of embodiments 1 to 10, wherein the spacer peptide lacks a plant endoproteinase recognition site.

13. The recombinant nucleic acid molecule of embodiment 12, wherein the spacer peptide forms a junction with each defensin peptide and each junction lacks a plant endoproteinase recognition site.

14. The recombinant nucleic acid molecule of embodiment 12 or 13, wherein the spacer peptide and the junctions are resistant to cleavage by a plant endoproteinase of a monocot crop plant.

15. The nucleic acid molecule of embodiment 14, wherein the monocot crop plant is selected from the group consisting of corn, barley, oat, rice, sorghum, sugarcane, pearl millet, turf grass, and wheat.

16. The recombinant nucleic acid molecule of any embodiment 12 or 13, wherein the spacer peptide and the junctions are resistant to cleavage by a plant endoproteinase of a dicot crop plant.

17. The nucleic acid molecule of embodiment 16, wherein the dicot crop plant is selected from the group consisting of alfalfa, Brassica sp., cotton, cucurbit, potato, strawberry, sugar beet, soybean, and tomato.

18. The recombinant nucleic acid molecule of any one of embodiments 1 to 17, wherein the spacer peptide is derived from a wild-type linker peptide and comprises at least one mutation that renders the mutated linker peptide resistant to cleavage by a plant endoproteinase in comparison to the corresponding wild-type linker peptide.

19. The recombinant nucleic acid molecule of embodiment 18, wherein the mutation comprises an amino acid residue insertion, deletion, substitution, or combination thereof relative to the corresponding wild-type linker peptide.

20. The recombinant nucleic acid molecule of embodiment 18, wherein the corresponding wild-type linker peptide is mutated at an amino acid residue selected from the group consisting of arginyl, lysyl, aspartyl, and glutamyl residue.

21. The recombinant nucleic acid molecule of any one of embodiments 18 to 20, wherein the mutation eliminates a diacidic amino acid sequence, a dibasic amino acid sequence, or both in the wild-type linker peptide.

22. The recombinant nucleic acid molecule of embodiment 21, wherein the mutation eliminating a diacidic amino acid sequence, a dibasic amino acid sequence, or both in the wild-type linker peptide comprises a substitution of at least one acidic or basic amino acid residue with a glycyl, serinyl, threonyl, alaninyl, leucinyl, isoleucinyl, valinyl, prolyl, phenylalaninyl, tryptophanyl, or methionyl residue.

23. The recombinant nucleic acid molecule of any one of embodiments 18 to 22, wherein the mutated linker peptide is resistant to cleavage by at least one of a cysteine, serine, threonine, metallo-, or aspartic plant endoproteinase.

24. The recombinant nucleic acid molecule of any one of embodiments 18 to 23, wherein the mutated linker peptide comprises a polypeptide sequence having at least one, two, three, four, or five amino acid insertions, substitutions, or deletions in SEQ ID NO:6, SEQ ID NO:60, SEQ ID NO:70, SEQ ID NO:74, the amino acid sequence alanyl-glycyl-threonyl, or SEQ ID NO:100.

25. The recombinant nucleic acid molecule of any one of embodiments 1 to 17, wherein the spacer peptide comprises a heterologous spacer peptide that comprises a SEQ ID NO:103; SEQ ID NO:104; SEQ ID NO:105, (Gly₄)n sequence, a (Gly₄Ser)n sequence of SEQ ID NO:93, a Ser(Gly₄Ser)n sequence of SEQ ID NO:94, or combination thereof, wherein n is a positive integer equal to 1, 2, 3, 4, 5, 6, 7, 8, 9 or 10.

26. The recombinant nucleic acid molecule of any one of embodiments 1 to 17, wherein the encoded spacer peptide is an encoded heterologous spacer peptide that is not operably associated with the encoded defensin peptides in a nucleic acid isolated from a naturally occurring host organism.

27. The recombinant nucleic acid molecule of any one of embodiments 1 to 26, wherein the two defensin peptides are both derived from a single defensin protein, a defensin proprotein, or variant thereof.

28. The recombinant nucleic acid molecule of any one of embodiments 1 to 26, wherein the two defensin peptides are derived from two distinct defensin proteins, two distinct defensin proproteins, or variants thereof.

29. The recombinant nucleic acid molecule of any one of embodiments 1 to 28, wherein the multimeric defensin polypeptide can specifically bind at least one phospholipid, at least one sphingolipid, or a combination thereof.

30. The recombinant nucleic acid of embodiment 29, wherein the phospholipid is selected from the group consisting of phosphatidic acid, a phosphatidylinositol monophosphate, a phosphatidylinositol bisphosphate, and combinations thereof and the sphingolipid is glucosylceramide.

31. The recombinant nucleic acid of embodiment 30, wherein the phosphatidyl inositol monophosphate is phosphatidylinositol 3-phosphate, phosphatidylinositol 4-phosphate, phosphatidylinositol-5 phosphate, or any combinations thereof.

32. The recombinant nucleic acid of anyone of embodiments 29 to 31, wherein binding of the multimeric defensin polypeptide to the phospholipid is increased in comparison to binding of a polypeptide consisting of a single defensin peptide of the multimeric defensin polypeptide to the phospholipid.

33. The recombinant nucleic acid molecule of any one of embodiments 1 to 32, wherein at least one of the defensin peptides comprises an amino acid sequence at least 70%, 75%, 80%, 85%, 90%, 95%, 98%, 99%, or 100% identical to SEQ ID NO:4, SEQ ID NO:5, SEQ ID NO:7, SEQ ID NO:38, SEQ ID NO:40, SEQ ID NO:55, SEQ ID NO:59, SEQ ID NO:61, SEQ ID NO:62, SEQ ID NO:76, SEQ ID NO:77, SEQ ID NO:79, SEQ ID NO:81, SEQ ID NO:83, SEQ ID NO:96, SEQ ID NO:97, SEQ ID NO:99, SEQ ID NO:101, SEQ ID NO:108, SEQ ID NO:109, SEQ ID NO:110, SEQ ID NO:117, SEQ ID NO:118, SEQ ID NO:119, SEQ ID NO:120, functional fragments thereof, and chimeras thereof.

34. The recombinant nucleic acid molecule of any one of embodiments 1 to 32, wherein the two defensin peptides are the same or different and each comprises an amino acid sequence at least 70%, 75%, 80%, 85%, 90%, 95%, 98%, 99%, or 100% identical to an amino acid sequence independently selected from the group consisting of SEQ ID NO:4, SEQ ID NO:5, SEQ ID NO:7, SEQ ID NO:38, SEQ ID NO:40, SEQ ID NO:55, SEQ ID NO:59, SEQ ID NO:61, SEQ ID NO:62, SEQ ID NO:76, SEQ ID NO:77, SEQ ID NO:79, SEQ ID NO:81, SEQ ID NO:83, SEQ ID NO:96, SEQ ID NO:97, SEQ ID NO:99, SEQ ID NO:101, SEQ ID NO:108, SEQ ID NO:109, SEQ ID NO:110, SEQ ID NO:117, SEQ ID NO:118, SEQ ID NO:119, SEQ ID NO:120, functional fragments thereof, and chimeras thereof.

35. The recombinant nucleic acid molecule of any one of embodiments 1 to 32, wherein the defensin peptides are the same or different and each comprises an amino acid sequence at least 50%, 60%, 70%, 75%, 80%, 85%, 90%, 95%, 98%, 99%, or 100% identical to an amino acid sequence independently selected from the group consisting of SEQ ID NO:4, SEQ ID NO:5, SEQ ID NO:7, SEQ ID NO:38, SEQ ID NO:40, SEQ ID NO:55, SEQ ID NO:117, SEQ ID NO:118, functional fragments thereof, and chimeras thereof.

36. The recombinant nucleic acid molecule of embodiment 35, wherein: (i) the defensin peptides are the same or different and each comprises an amino acid sequence at least 84%, 86%, 88%, 90%, 92%, 95%, 98%, 99%, or 100% identical to an amino acid sequence independently selected from the group consisting of SEQ ID NO:4 , SEQ ID NO:5, SEQ ID NO:117, SEQ ID NO:118, functional fragments thereof, and chimeras thereof.

37. The recombinant nucleic acid molecule of embodiment 35 or 36, wherein binding of the encoded polypeptide multimeric defensin polypeptide to phosphatidylserine, phosphatidylinositol 3-phosphate, phosphatidylinositol 4-phosphate, phosphatidylinositol 5-phosphate, or any combination thereof is increased in comparison to binding of a polypeptide consisting of a single defensin peptide of the multimeric defensin polypeptide to phosphatidylserine, phosphatidylinositol 3-phosphate, phosphatidylinositol 4-phosphate, phosphatidylinositol 5-phosphate, or any combination thereof.

38. The recombinant nucleic acid molecule of any one of embodiments 1 to 37, wherein permeability of a fungal plasma membrane treated with the multimeric defensin polypeptide is increased in comparison to permeability of a fungal plasma membrane treated with single defensin peptide of the multimeric defensin polypeptide.

39. A transformed host cell comprising the recombinant nucleic acid molecule of any one of embodiments 1 to 38.

40. The host cell of embodiment 39, wherein the cell is a bacterial cell, a yeast cell, or a plant cell.

41. A transgenic plant comprising the recombinant nucleic acid molecule of any one of embodiments 1 to 37.

42. The transgenic plant of embodiment 41, wherein the recombinant nucleic acid molecule confers the plant with resistance to infection by a plant pathogenic fungus in comparison to a control plant that lacks the recombinant nucleic acid molecule.

43. The transgenic plant of embodiment 41 or 42, wherein a plant pathogenic fungus inhibitory amount of the multimeric defensin polypeptide is an amount of at least 0.005, 0.05, 0.5, or 1 PPM in a tissue or part of the plant.

44. The transgenic plant of embodiment 41, wherein the plant pathogenic fungus is a Fusarium sp., Alternaria sp., Verticillium dahlia, Phytophthora sp., Colletotricum sp., Botrytis cinerea, Cercospora sp., or Puccinia sp.

45. The transgenic plant of any one of embodiments 41 to 44, wherein the plant is a monocot crop plant or a dicot crop plant.

46. The transgenic plant of embodiment 45, wherein the monocot crop plant is selected from the group consisting of a corn, barley, oat, pearl millet, rice, sorghum, sugarcane, turf grass, and wheat.

47. The transgenic plant of embodiment 45, wherein the dicot crop plant is selected from the group consisting of alfalfa, a Brassica sp., cotton, cucurbit, potato, strawberry, sugar beet, soybean, and tomato.

48. A transgenic plant part of the transgenic plant of any one of embodiments 41 to 47, wherein the plant part comprises the recombinant nucleic acid.

49. The transgenic plant part of embodiment 48, wherein the part is selected from the group consisting of a flower, leaf, root, seed, stem, and a tuber.

50. A processed plant product of the transgenic plant part of any one of embodiments 41 to 49, wherein the processed plant product comprises the recombinant nucleic acid or a fragment thereof.

51. The processed plant product of embodiment 50, wherein the product is non-regenerable.

52. The processed plant product of embodiment 50, wherein the product is a meal or flour.

53. The processed plant product of any one of embodiments 51 or 52, wherein the fragment comprises a recombinant polynucleotide encoding: (i) at least one junction of a defensin peptide with the heterologous spacer peptide; (ii) a junction of a heterologous localization peptide with a defensin peptide, spacer peptide, or any combination thereof and (iii) a junction of a heterologous promoter to a defensin peptide.

54. The processed plant product of any one of embodiments 51 to 53, wherein the processed plant product is characterized by having reduced levels of fungal toxins in comparison to processed plant products obtained from corresponding control plant crops.

55. A method for obtaining a transgenic plant that is resistant to infection by a plant pathogenic fungus comprising the steps of: (i) introducing the recombinant nucleic acid molecule of any one of embodiments 1 to 38 into a plant cell, tissue, part, or whole plant; (ii) obtaining a plant cell, tissue, part, or whole plant into which the recombinant nucleic acid molecule has integrated into the plant nuclear or plastid genome; and (iii) selecting a transgenic plant obtained from the plant cell, tissue, part or whole plant of step (ii) for expression of a plant pathogenic fungus inhibitory amount of the multimeric defensin polypeptide, thereby obtaining a transgenic plant that is resistant to infection by a plant pathogenic fungus.

56. The method of embodiment 55, wherein the recombinant nucleic acid molecule is introduced into the plant cell, tissue, part, or whole plant by Agrobacterium or particle-mediated transformation.

57. The method of embodiment 55, wherein the recombinant nucleic acid molecule is introduced in step (i) with a clustered regularly interspaced short palindromic repeats (CRISPR)-associated (Cas)-guide RNA or source thereof and a Cas endonuclease or source thereof, wherein the guide RNA and Cas endonuclease can form a complex that can introduce a double strand break at a target site in a nuclear genome of the plant cell, tissue, part, or whole plant.

58. A method for obtaining a transgenic plant that is resistant to infection by a plant pathogenic fungus comprising the steps of: (i) providing a clustered regularly interspaced short palindromic repeats (CRISPR)-associated (Cas)-guide RNA, a Cas endonuclease, and a DNA molecule comprising a polynucleotide encoding at least one of a defensin peptide, a spacer peptide that is resistant to cleavage by a plant endoproteinase, a heterologous promoter, or a heterologous localization peptide, to a plant cell, tissue, part, or whole plant, wherein the guide RNA and Cas endonuclease can form a complex that can introduce a double strand break at a target site in a nuclear or plastid genome of the plant cell, tissue, part, or whole plant; (ii) obtaining a plant cell, tissue, part, or whole plant wherein the DNA molecule has integrated into the target site; and (iii) selecting a transgenic plant obtained from the plant cell, tissue, part or whole plant of step (ii) comprising the recombinant nucleic acid molecule of any one of embodiments 1 to 38 for expression of a plant pathogenic fungus inhibitory amount of the multimeric defensin polypeptide, thereby obtaining a transgenic plant that is resistant to infection by a plant pathogenic fungus.

59. The method of embodiment 58, wherein the polynucleotide in step (i) encodes a defensin peptide that is operably associated with a spacer peptide.

60. A method for producing transgenic plant seed that provide plants resistant to infection by a plant pathogenic fungus that comprises the steps of: (i) selfing or crossing the transgenic plant of any one of embodiments 41 to 47; and (ii) harvesting seed that comprises the recombinant nucleic acid molecule of the transgenic plant from the self or cross, thereby producing transgenic plant seed that provide plants resistant to infection by a plant pathogenic fungus.

61. The method of embodiment 60, wherein the transgenic plant is used as a pollen donor in the cross and the seed are harvested from a pollen recipient.

62. A method for reducing crop damage by a plant pathogenic fungus comprising the steps of: (i) placing seeds or cuttings of the transgenic plants of any one of embodiments 41 to 47 in a field where control plants are susceptible to infection by at least one plant pathogenic fungus; and (ii) cultivating a crop of plants from the seeds or cuttings, thereby reducing crop damage by the plant pathogenic fungus.

63. The method of embodiment 62, wherein the method further comprises the step of harvesting seed, fruit, leaves, tubers, stems, roots, or any combination thereof from the crop.

64. The method of embodiment 63, wherein said seed, fruit, leaves, tubers, stems, roots, or any combination thereof have reduced levels of fungal toxins in comparison to seed, fruit, leaves, tubers, stems, roots, or any combination thereof obtained from corresponding control plant crops.

65. The method of any one of embodiments 62 to 64, wherein the plant pathogenic fungus is a Fusarium sp., Alternaria sp., Verticillium dahlia, Phytophthora sp., Colletotricum sp., Botrytis cinerea, Cercospora sp., or Puccinia sp.

66. A method for obtaining a genetically edited plant expressing a multimeric defensin comprising an amino-terminal first defensin peptide, a mutagenized linker peptide, and a carboxy-terminal second defensin peptide comprising the steps of: (i) introducing a clustered regularly interspaced short palindromic repeats (CRISPR)-associated (Cas)-guide RNA, a Cas endonuclease, and a mutagenizing polynucleotide capable of converting an endogenous wild-type genomic DNA encoding a linker peptide that is susceptible to cleavage by a plant endoproteinase to a mutagenized genomic DNA encoding a spacer peptide that is resistant to cleavage by a plant endoproteinase, into a wild-type plant cell, tissue, part, or whole plant; (ii) obtaining a plant cell, tissue, part, or whole plant wherein the nuclear genome has been mutagenized; and (iii) selecting a genetically edited plant obtained from the plant cell, tissue, part or whole plant of step (iii) for expression of a multimeric defensin comprising the first defensin peptide, the mutagenized linker peptide, and the second defensin peptide.

67. The method of embodiment 66, wherein the genetically edited plant exhibits improved resistant to infection by a plant pathogenic fungus when compared to a control plant having the wild-type linker peptide domain.

68. The method of embodiment 67, wherein a wild-type linker peptide comprising an amino acid sequence that is diacidic, dibasic, or a combination thereof is converted to an amino acid sequence that lacks the diacidic, dibasic, or both the diacidic and dibasic amino acid sequences.

69. A genetically edited plant made by the method of any one of embodiments 66 to 68 or progeny thereof that comprise the mutagenized genomic DNA encoding the spacer peptide that is resistant to cleavage by a plant endoproteinase.

70. A genetically edited seed obtained from the plant of embodiment 69, said seed comprising the mutagenized genomic DNA encoding the spacer peptide that is resistant to cleavage by a plant endoproteinase.

71. An isolated polypeptide encoded by the recombinant nucleic acid of any one of embodiments 1 to 38.

72. A method of producing a polypeptide encoded by the recombinant nucleic acid molecule of any one of embodiments 1 to 38, comprising culturing the host cell of embodiment 39 or 40 under conditions in which the polypeptide encoded by the polynucleotide is expressed, and recovering the polypeptide.

73. A composition comprising the isolated polypeptide of embodiment 71 and a carrier.

74. A method for controlling fungal infection comprising the step of applying an effective amount of the isolated polypeptide of embodiment 71 or the composition of 73 to a subject in need thereof, a plant, a plant part, or a processed plant product.

75. The method of embodiment 74, wherein the fungal infection is caused by a Fusarium sp., Alternaria sp., Verticillium dahlia, Phytophthora sp., Colletotricum sp., Botrytis cinerea, Cercospora sp., or Puccinia sp.

76. Use of the recombinant nucleic acid of any one of embodiments 1 to 38, the transformed host cell of embodiments 39 or 40, the transgenic plant of any one of embodiments 41 to 47, the transgenic plant part of any one of embodiments 48 to 49, the processed plant product of any one of embodiments 50 to 54, the polypeptide of embodiment 71, the genetically edited plant of any one of embodiments 66 to 69, the genetically edited seed of embodiment 70, or the composition of embodiment 73 to inhibit growth of a susceptible fungal species.

77. The use of embodiment 76, wherein the susceptible fungal species is a Fusarium sp., Alternaria sp., Verticillium dahlia, Phytophthora sp., Colletotricum sp., Botrytis cinerea, Cercospora sp., or Puccinia sp.

### OTHER PUBLICATIONS CITED

Argos, (1990) J Mol Biol. Feb 20; 211(4):943-58.
Baron, O. L. and Pauron, D. (2014). Bio-protocol 4(18): e1237.
Bent A F, et al., (1994) Science 265(5180): 1856-60.
Broekaert, W. F., et al., (1990). FEMS Microbiology Letters 69, 55-60.
Broekaert, W. F., et al., (1995). Plant Physiol 108, 1353-1358.
Broekaert, W. F., et al., (1997). Critical Reviews in Plant Sciences 16, 297-323.
Broothaerts W, et al., (2005) Nature. 433(7026):629-33.
Bustin, S. A. (2002) Journal of Molecular Endocrinology 29, 23-39.
Callis, J, Fromm, M, Walbot, V. (1987) Genes Dev. 1987 December; 1(10): 1 183-200.
Cappelini, R. A., and Peterson, J. L. (1965) Mycologia 57, 962-966.
Carrie and Small (2013) Biochimica et Biophysica Acta (BBA) Molecular Cell Research, 1833, (2), 253-259.
Cazzonnelli, C. I. and J. Velten. (2003) Plant Molecular Biology Reporter 21 : 271-280.
Collier, R., et al., (2005) Plant J 43: 449-457.
Chen et al., (2013) Adv Drug Deliv Rev.; 65(10): 1357-1369.
Correll, J. C., et al., (1987). Phytopathology 77, 1640-1646.
da Silva Conceicao, A., and Broekaert, W. F. (1999). Plant Defensins. In Pathogenesis-related proteins in plants, S. Muthukrishnan, ed (New York: CRC Press), pp. 247-260.
Davidson et al., (2006) Lipid Research, 47, 440-449.
Dowler et al.; Sci STKE. 2002 Apr. 23; 2002(129):16.
Doyle J J, et al., (1986). J Biol Chem. 261(20):9228-38.
François et al., Plant Physiology (2002) 128: 1346-1358.
Frame, B. R., et al., (2002) Plant Physiol. 129(1): 13-22.
Gao, A., et al., (2000). Nature Biotechnology 18, 1307-1310.
George R A, and Heringa (2002) J Protein Eng. 15(11):871-879.
Grant M R, et al., (1995) Science. 269(5225):843-6.
Hammond-Kosack, K. E., Urban, M., Baldwin, T., Daudi, A., Rudd, J. J., Keon, J., Lucas, J. A., Maguire, K., Kornyukhin, D., Jing, H.-C, Bass, C, and Antoniw, J. (2004). 4th International Crop Science Congress. In New directions for a diverse planet, T. Fischer, Turner, N., Angus, J., McIntyre, L., Robertson, M., Borrell, A., Lloyd, D., ed (Brisbane, Australia: The Regional Institute, Ltd, Gosford, Australia).
Hanks, J. N., et al., (2005). Plant Mol Biol 58, 385-399.
Horsch, R. B., et al., (1985) Science. 227: 1229-1231.
Huang et al., (2009) Plant Physiology, 150(3): 1272-1285.
Kingsman S M, et al., (1985) Biotechnol Genet Eng Rev. 3:377-416.
Koehler S M, and Ho, T H. (1990) Plant Cell. (8):769-83.
Kumar, V. and Jain, M. (2015) J Exp Bot 66: 47-57.
Lam E, and Chua N H.(1991). J Biol Chem. 1991 Sep. 15; 266(26): 17131-5.
Lacerda et al., Frontiers in Microbio. (2014) 5(116):1-10.
Lay, F. T., and Anderson, M. A. (2005) Curr Protein Pept Sci 6, 85-101.
Lee et al., (2015) Plant Physiol. 169(1):471-84.
Li and Teng (2013) Trends Plant Sci 18: 360-366.
Liang, J., Shah, D. M., Wu, Y., Rosenberger, C. A., and Hakimi, S. M. U.S. Pat. No. 6,916,970; issued Jul. 12, 2005.
Mankin, S. L, G. C. Allen, and W. F. Thompson. 1997. Plant Mol Biol Rep 15(2): 186-196.
McElroy, D, et al., (1990) The Plant Cell, Vol. 2, 163-171.
Miller and Cistola (1993) Molecular and Cellular Biochemistry, 123(1): 29-37.
Mottram et al., FEBS Lett. (1989) 258(2):211-215.
Reiser J, et al., (1990) Adv Biochem Eng Biotechnol.;43:75-102.
Sagaram et al., (2011) PLOS ONE 6: e18550.
Sagaram et al., (2013) PLoS ONE, 8(12): e82485.
Sambrook, J., and Russell, D. W. (2001). Molecular Cloning: A Laboratory Manual. (Cold Spring Harbor Laboratory Press, Cold Spring Harbor, New York).
Seong, K., et al., (2005) Phytopathology 95, 744-750.
Sjoling and Glaser; Trend Plant Sci., 1998, 3 (4) 136-140.
Sidorov, V, et al., (2006). Plant Cell Rep. 2006 April; 25(4):320-8. (Epub 2005 Oct. 27).
Svitashev et al., (2015) Plant Physiology, 169 (2): 931-945.
Spelbrink, R. G., et al., (2004). Plant Physiol 135, 2055-2067.
Terras, F. R., et al., (1992). J Biol Chem 267, 15301-15309.
Thevissen, K., et al., (2005). Curr Drug Targets 6, 923-928.
Thevissen, K., et al., (1996). J Biol Chem 271, 15018-15025.
Thevissen, K., et al., (2000). Proc Natl Acad Sci U S A 97, 9531-9536.
Thevissen, K., et al., (2004). J Biol Chem 279, 3900-3905.
Thomma, B. P., et al., (2003). Curr Drug Targets Infect Disord 3, 1-8.
Thomma, B. P. H. J. , Camrnue, B. P. A., and Thevissen, K. (2002). Planta 216, 193-202.
Turner et al., 1993) Protein Eng. 6(1):101-108.
Voytas, D. Annual Review of Plant Biology, Vol. 64: 327-350, 2013.
Tsiatsiani et al., (2012) Physiologia Plantarum 145: 28-40.
Using Antibodies: A Laboratory Manual.(1999). Ed. Harlow and Lane. Cold Spring Harbor Laboratory Press.
Vasil V, et al., (1989) Plant Physiol. 1989 December; 91(4): 1575-1579.
Vasivarama and Kirti (2013a) Plant Cell Tiss Organ Cult, 115:309-319.
Vasivarama and Kirti (2013b) Funct Integr Genomics 13:435-443.
Wesley S V, (2001). Plant J. 27(6):581-590.

1.-77. (canceled)

78. A method of obtaining an optimized multimeric defensin (MD) protein with improved antifungal activity comprising:
(i) assaying one or more MD proteins comprising a first defensin peptide which is operably linked with a spacer peptide to a second defensin peptide for improved phospholipid and/or sphingolipid binding in comparison to a reference protein or for increased protein oligomerization in the presence of a phospholipid and/or sphingolipid in comparison to a reference protein; and
(iii) selecting an optimized MD protein that exhibits the improved phospholipid and/or sphingolipid binding or increased protein oligomerization, thereby obtaining an optimized MD protein with improved antifungal activity.

79. The method of claim 78, wherein the first and second defensin peptides in the MD protein are identical or related to one another such that the two peptides have at least 60%, 70%, 80%, 85%, 90%, 95%, 98%, or 99% sequence identity.

80. The method of claim 78, wherein the first and second defensin peptides in the MD protein are distinct and have less than 60% identity to one another.

81. The method of claim 78, wherein the first and second defensin peptides can comprise any combination of MtDef4, MsDef1, NaD1, TPP3, MtDef5, RsAFP2, DmAMP1, Psd1 defensin peptides or variants thereof.

82. The method of claim 78, wherein the spacer peptide is selected from a wild type or mutagenized linker peptide that joins defensin peptides, a spacer peptide from a multimeric- or multi-domain protein that does not contain defensin peptides, or a wholly or partially synthetic peptide sequence.

83. The method of claim 82, wherein the spacer peptide comprises the MtDef5 linker peptide of SEQ ID NO:6 or a mutant thereof comprising at least one amino acid insertion or substitution.

84. The method of claim 78, wherein the optimized MD protein is selected for increased oligomerization in the presence of a phospholipid in comparison to the reference protein.

85. The method of claim 78, wherein oligomerization is assayed by incubating the MD protein and reference protein with a phospholipid and/or sphingolipid, crosslinking the proteins, reducing the proteins, and separating the proteins by denaturing polyacrylamide gel electrophoresis.

86. The method of claim 78, wherein the optimized MD protein is further selected for increased fungal plasma membrane permeabilization activity in comparison to the reference protein.

87. The method of claim 85, wherein fungal plasma membrane permeabilization activity is measured by dye uptake in a fungal cell treated with the MD protein and in a fungal cell treated with the reference protein.

88. The method of claim 78, wherein phospholipid and/or sphingolipid binding is assayed with a protein-lipid overlay assay, surface plasmon resonance assay, a biotin capture lipid affinity assay, or a titration calorimetry assay.

89. The method of claim 78, wherein the reference protein is one of the defensin peptides in the MD protein or a control MD protein.

90. The method of claim 78, wherein the optimized MD exhibits a lower IC50 value against one or more fungal pathogens in comparison to the reference protein.

91. The method of claim 90, wherein the fungal pathogen is a plant pathogenic Fusarium sp., Alternaria sp., Verticillium dahlia, Phytophthora sp., Phakopsora pachyrhizi, Sclerotinia sp., Phialophora gregata, Colletotricum sp., Botrytis cinerea, Cercospora sp., or Puccinia sp.
